(19) United States
(12) Patent Application Publication
(10) Pub. No.: US 2009/0258907 A1
(43) Pub. Date: Oct. 15, 2009

(54) COMPOUNDS USEFUL AS INHIBITORS OF ROCK KINASES

(75) Inventors: Steven L. Swann, Antioch, IL (US); Anil Vasudevan, Union Grove, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/419,765

(22) Filed: Apr. 7, 2009

Related U.S. Application Data
(60) Provisional application No. 61/043,632, filed on Apr. 9, 2008.

(51) Int. Cl. A61K 31/4439; C07D 417/04; A61P 9/10

(52) U.S. Cl. 514/338; 546/270.1

(57) ABSTRACT

Disclosed herein are compounds of formula (I) or pharmaceutical acceptable salts thereof,

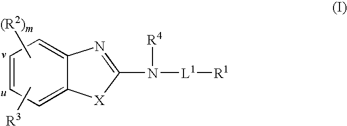

wherein X, R¹, R², R³, R⁴, L¹, and m, are defined in the specification. Compositions comprising said compounds which can be useful for inhibiting Rho kinase (ROCK) and methods for using said compositions are also described.

This application claims priority to U.S. patent application Ser. No. 61/043,632, filed Apr. 9, 2008, and is incorporated herein by reference.

### TECHNICAL FIELD

Bicyclic compounds that are inhibitors of Rho kinases (ROCK), compositions comprising such compounds, and methods of treating conditions and disorders using such compounds and compositions are provided.

### BACKGROUND

An important large family of enzymes is the protein kinase enzyme family. Currently, there are about 500 different known protein kinases. Protein kinases serve to catalyze the phosphorylation of an amino acid side chain in various proteins by the transfer of the y-phosphate of the ATP-Mg²⁺ complex to said amino acid side chain.

These enzymes control the majority of the signalling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied families of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium- and phospholipid-dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases.

Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also in progress to identify modulators of tyrosine kinases as well.

A major signal transduction system utilized by cells is the RhoA-signalling pathway. RhoA is a small GTP binding protein that can be activated by several extracellular stimuli such as growth factor, hormones, mechanic stress, or osmotic change as well as high concentration of metabolite like glucose. RhoA activation involves GTP binding, conformation alteration, post-translational modification (geranylization and farnesylation) and activation of its intrinsic GTPase activity. Activated RhoA is capable of interacting with several effector proteins including ROCKs (Rho kinase) and transmit signals into cellular cytoplasm and nucleus.

Rho kinase is found in two isoforms encoded by two different genes of ROCK, ROCK 1 (also known as ROCKβ or p160-ROCK) and ROCK 2 (also known as ROCKα). Both ROCK 1 and ROCK 2 contain an amino-terminal catalytic kinase domain, a central coiled-coil domain of about 600 amino acids, and a carboxyl-terminal pleckstrin homology (PH) domain that is split by a cysteine-rich region. Rho/GTP interacts with the C-terminal portion of the central coiled-coil domain and activates the kinase activity of ROCK.

Thus, ROCK1 and 2 constitute a family of serine/threonine kinases that can be activated by RhoA-GTP complex via physical association. Activated ROCKs phosphorylate a number of substrates and play important roles in pivotal cellular functions. The substrates for ROCKs include myosin binding subunit of myosin light chain phosphatase (MBS, also named MYPT1), adducin, moesin, myosin light chain (MLC), LIM kinase as well as transcription factor FHL. The phosphorylation of theses substrates modulate the biological activity of the proteins and thus provide a means to alter cell's response to external stimuli. One well documented example is the participation of ROCK in smooth muscle contraction. Upon stimulation by phenylephrine, smooth muscle from blood vessels contracts. Studies have shown that phenylephrine stimulates alpha-adrenergic receptors and leads to the activation of RhoA. Activated RhoA in turn stimulates kinase activity of ROCK1 and which in turn phosphorylates MBS. Such phosphorylation inhibits the enzyme activity of myosin light chain phosphatase and increases the phosphorylation of myosin light chain itself by a calcium-dependent myosin light chain kinase (MLCK) and consequently increases the contractility of myosin-actin bundle, leading to smooth muscle contraction. This phenomenon is also sometimes called calcium sensitization. In addition to smooth muscle contraction, ROCKs have also been shown to be involved in cellular functions including apoptosis, cell migration, transcriptional activation, fibrosis, cytokinesis, inflammation and cell proliferation. Moreover, in neurons ROCK plays a critical role in the inhibition of axonal growth by myelin-associated inhibitory factors such as myelin-associated glycoprotein (MAG). ROCK-activity also mediates the collapse of growth cones in developing neurons. Both processes are thought to be mediated by ROCK-induced phosphorylation of substrates such as LIM kinase and myosin light chain phosphatase, resulting in increased contractility of the neuronal actin-myosin system.

Abnormal activation of the Rho/ROCK pathway has been observed in various disorders (¹Wettschureck, N., Offermanns, S., Rho/Rho-kinase mediated signaling in physiology and pathophysiology. J. Mol. Med. 80, 2002, 629-638; ²Müller, B. K., Mack, H., Teusch, N., Rho kinase, a promising drug target for neurological discorders. Nat. Drug Discov. Rev. 4, 2005, 387-398; ³Hu, E, Lee, D., ROCK inhibitors as potential therapeutic agents for cardiovascular diseases. Curr. Opin. Investig. Drugs. 4, 2003, 1065-1075). As already mentioned, ROCKs phosphorylate the myosin binding subunit of myosin light chain (MLC) phosphatase (MLCP), resulting in increased myosin phosphorylation and actin-myosin contraction (⁴Somlyo, A. P., Somlyo, A. V., Ca2+ sensitivity of smooth muscle and nonmuscle myosin II: modulated by G proteins, kinases, and myosin phosphatase. Physiol. Rev. 83, 2003, 1325-1358). Examples of disease states related with abnormal Rho/ROCK activity, in particular with vasospasm activity, include cardiovascular diseases such as hypertension (⁹Satoh S., Kreutz R., Wilm C., Ganten D., Pfitzer G., Augmented agonist-induced Ca²⁺-sensitization of coronary artery contraction in genetically hypertensive rats. Evidence for altered signal transduction in the coronary smooth muscle cells. J. Clin. Invest. 94, 1994, 1397-1403; ¹⁰Mukai, Y., Shimokawa, H., Matoba, T., Kandabashi, T., Satoh, S., Hiroki. J., Kaibuchi, K., Takeshita, A., Involvement of Rho-kinase in hypertensive vascular disease: a novel therapeutic target in hypertension. FASEB J. 15, 2001,1062-1064; ¹¹Uehata, M., Ishizaki, T., Satoh, H., Ono, T., Kawahara, T., Morishita, T., Tamakawa, H., Yamagami, K., Inui, J., Maekawa, M., Narumiya, S., Calcium sensitization of smooth muscle mediated by a Rho-associated protein kinase in hypertension. Nature 389, 1997, 990-994; ¹²Masumoto, A., Hirooka, Y., Shimokawa, H., Hironaga, K., Setoguchi, S., Takeshita, A., Possible involvement of Rhokinase in the pathogenesis of hypertension in humans. Hypertension 38, 2001, 1307-1310), chronic and congestive heart failure (¹⁸Fuster, V., Badimon, L., Badimon, J J, Chesebro, J H, The pathogenesis of coronary artery disease and the acute coronary syndromes (2). N Engl J Med 326, 1992, 310-318; ¹⁹Shimokaw a, H., Cellular and molecular mechanisms of coronary artery spasm: lessons from animal models. Jpn Circ J 64, 2000, 1-12; ²⁰Shimokawa, H., Morishige, K., Miyata, K., Kandabashi, T., Eto, Y., Ikegaki, I., Asano, T., Kaibuchi, K., Takeshita, A., Longterm inhibition of Rho-kinase induces a regression of arteriosclerotic coronary lesions in a porcine model in vivo. Cardiovasc Res 51, 2001, 169-177; ²¹Utsunomiya, T., Satoh, S., Ikegaki, I., Toshima, Y., Asano, T., Shimokawa, H., Antianginal effects of hydroxyfasudil, a Rho-kinase inhibitor, in a canine model of effort angina. Br J Pharmacol 134, 201, 1724-1730), cardiac hypertrophy (⁴⁰Hoshijima, M., Sah, V. P., Wang, Y., Chien, K. R., Brown. J. H. The low molecular weight GTPase Rho regulates myofibril formation and organization in neonatal rat ventricular myocytes. Involvement of Rho kinase. J Biol Chem 273, 1998. 7725-77230; ⁴¹Sah, V. P., Hoshijima, M., Chien, K. R., Brown, J. H., Rho is required for Galphaq and alpha1-adrenergic receptor signal-637 ing in cardiomyocytes. Dissociation of Ras and Rho pathways, J Biol Chem 271, 1996, 31185-1190; ⁴²Kuwahara, K., Saito, Y., Nakagawa, O., Kishimoto, I., Harada, M., Ogawa, E., Miyamoto, Y., Hamanaka, I., Kajiyama, N., Takahashi, N., Izumi, T., Kawakami, R., Tamura, N., Ogawa, Y., Nakao, K. The effects of the selective ROCK inhibitor, Y27632, on ET-1-induced hypertrophic response in neonatal rat cardiacmyocytes-possible involvement of Rho/ROCK pathway in cardiac muscle cell hypertrophy. FEBS Lett 452, 1999, 314-318), chronic renal failure (⁷Sharpe, C. C., Hendry, B. M. Signaling: focus on Rho in renal disease. J. Am. Soc. Nephrol. 14, 2003, 261-264), cerebral vasospasm after subarachnoid bleeding (¹³Shibuya, M., Suzuki, Y., Sugita, K., Saito, I., Sasaki, T., Takakura, K., Okamoto, S., Kikuchi, H., Takemae, T., Hidaka, H. Dose escalation trial of a novel calcium antagonist, AT877, in patients 636 with aneurysmal subarachnoid haemorrhage. Acta Neurochir (Wien) 107, 1990, 11-15; ¹⁴Shibuya, M., Suzuki, Y., Sugita, K., Saito, I., Sasaki, T., Takakura, K., Nagata, I., Kikuchi, H., Takemae, T., Hidaka, H., et. al. Effect of AT877 on cerebral vasospasm after aneurysmal subarachnoid hemorrhage. Results of a prospective placebo-controlled double-blind trial. J Neurosurg 76, 1992, 571-577; ¹⁵Sato, M., Tani, E., Fujikawa, H., Kaibuchi, K., Involvement of Rho-kinase-mediated phosphorylation of myosin light chain in enhancement of cerebral vasospasm. Circ Res 87, 2000, 195-200; ¹⁶Miyagi, Y., Carpenter, R. C., Meguro, T., Parent, A. D., Zhang, J. H., Upregulation of rho A and rho kinase messenger RNAs in the basilar artery of a rat model of subarachnoid hemorrhage. J Neurosurg 93, 2000, 471-476; ¹⁷Tachibana, E., Harada, T., Shibuya, M. Saito, K., Takayasu, M., Suzuki, Y., Yoshida, J., Intra-arterial infusion of fasudil hydrochloride for treating vasospasm following subarachnoid haemorrhage. Acta Neurochir (Wien) 141, 1999, 13-19), pulmonary hypertension (⁵Sylvester, J. T., Am. J. Physiol. Lung Cell. Mol. Physiol. 287, 2004, L624-L630) and ocular hypertension (³⁴Honjo, M., Inatani, M., Kido, N., Sawamura, T., Yue, B. Y., Honda, Y., Tanihara, H., Effects of protein kinase inhibitor. HA1077, on intraocular pressure and outflow facility in rabbit eyes. Arch Ophthalmol 119, 2001, 1171-1178; ³⁵Rao, P. V, Deng, P. F., Kumar, J. Epstein, D. L., Modulation of aqueous humor outflow facility by the Rho kinase-specific inhibitor Y-27632. Invest Ophthalmol Vis Sci 42, 2001, 1029-1037). Further diseases related to abnormal Rho/ROCK activity are cancer (⁶Aznar, S., Fernandez-Valeron, P., Espina, C., Lacal, J. C., Rho GTPases: potential candidates for anticancer therapy. Cancer Lett. 206, 2004, 181-191; ⁴³Yin, L. et al., Fasudil inhibits vascular endothelial growth factor-induced angiogenesis in vitro and in vivo. Mol Cancer Ther 5, 2007, 1517-25; ⁴⁴Itoh, K., Yoshioka, K., Akedo, H., Uehata, M., Ishizaki, T., Narumiya, S., An essential part for Rho-associated kinase in the transcellular invasion of tumor cells. Nat Med 5, 1999, 221-225; ⁴⁵Genda, T. Sakamoto, M., Ichida, T., Asakura, H., Kojiro, M., Narumiya, S., Hirohashi, S., Cell motility mediated by rho and Rho-associated protein kinase plays a critical role inintrahepatic metastasis of human hepatocellular carcinoma. Hepatology 30, 1999, 1027-1036; ⁴⁶Somlyo, A. V., Bradshaw, D., Ramos, S., Murphy, C., Myers, C. E., Somlyo, A. P., Rho-kinase inhibitor retards migration and in vivo dissemination of human prostate cancer cells. Biochem Biophys Res Commun 269, 2000, 652-659), asthma (²⁴Roberts, J. A., Raeburn, D., Rodger, I. W., Thomson, N. C., Comparison of in vivo airway responsiveness and in vitro smooth muscle sensitivity to methacholine in man. Thorax 39; 1984, 837-843; ²⁵Chiba, Y., Misawa, M., Characteristics of muscarinic cholinoceptors in airways of antigen-induced airway hyperresponsive rats. Comp Biochem Physiol C Pharmacol Toxicol Endocrinol 111, 1995, 351-357; ²⁶Chiba, Y., Takada, Y., Miyamoto, S., MitsuiSaito, M., Karaki, H., Misawa, M., Augmented acetylcholine-induced, Rho mediated Ca²⁺ sensitization of bronchial smooth muscle contraction in antigen-induced airway hyperresponsive rats. Br J Pharmacol 127, 1999, 597-600; ²⁷Chiba, Y., Sakai, H. Misawa, M., Augmented acetylcholine-induced translocation of RhoA in bronchial smooth muscle from antigen-induced airway hyperresponsive rats. BrJ Pharmacol 133, 2001, 886-890; ²⁸Iizuka, K., Shimizu, Y., Tsukagoshi, H., Yoshii, A., Harada, T. Dobashi, K., Murozono, T., Nakazawa, T., Mori, M., Evaluation of Y-27632, a rho-kinase inhibitor, as a bronchodilator in guinea pigs. Eur J Pharmacol 406, 2000, 273-279), male erectile dysfunctions (⁸Andersson. K. E., Hedlund, P., New directions for erectile dysfunction therapies. Int. J. Impot. Res. 14 (Suppl. 1), 2002, S82-S92; ³²Chitaley, K., Wingard, C. J., Clinton Webb, R., Branam, H., Stopper, V. S., Lewis, R. W., Mills, T. M., Antagonism of Rho-kinase stimulates rat penile erection via a nitric oxideindependent pathway. Nat Med 7, 2001, 119-122; ³³Mills, T. M., Chitaley, K., Wingard, C. J., Lewis, R. W., Webb, R. C., Effect of Rho-kinase inhibition on vasoconstriction in the penile circulation. J Appl Physiol 91, 2001, 1269-1273), female sexual dysfunction, over-active bladder syndrome (⁶⁴Peters, S. L. et al., Rho kinase: a target for treating urinary bladder dysfunction. Trends Pharmacol Sci. 27, 2006, 492-7) and preterm labor (²⁹Niiro, N., Nishimura, J., Sakihara, C., Nakano, H., Kanaide, H., Up-regulation of rho A and rho-kinase mRNAs in the rat myometrium during pregnancy. Biochem Biophys Res Commun 230, 1997, 356-359; ³⁰Tahara, M., Morishige, K., Sawada, K., Ikebuchi, Y., Kawagishi, R., Tasaka, K., Murata, Y., RhoA/Rho-kinase cascade is involved in oxytocin-induced rat uterine contraction. Endocrinology 143, 2002, 920-929; ³¹Kupittayanant, S., Burdyga, T., Wray, S., The effects of inhibiting Rho-associated kinase with Y-27632 on force and intracellular calcium in human myometrium. Pflugers Arch. 443, 2001, 112-114).

Inhibitors of ROCKs have been suggested for use in the treatments of a variety of diseases. They include cardiovascular diseases such as hypertension (see above ⁹⁻¹²), chronic and congestive heart failure¹⁸⁻²¹, and cardiac hypertrophy⁴⁰⁻⁴², chronic renal failure⁷, furthermore cerebral vasospasm after subarachnoid bleeding¹³⁻¹⁷, pulmonary hypertension⁵ and ocular hypertension^{34, 35}. In addition, because of their muscle relaxing properties, they are also suitable for asthma²⁴⁻²⁸, male erectile dysfunctions^{8, 32, 33}, female sexual dysfunction and over-active bladder syndrome⁶⁴ and preterm labor²⁹⁻³¹. Several recent studies have reported the beneficial effects of ROCK inhibitors in ischemia-reperfusion and myocardial infarction. In these studies, the ROCK inhibitors Y-27632 and fasudil were shown to decrease ischemia-reperfusion injury, myocardial infarct size, and myocardial fibrosis in response to experimental myocardial infarction (MI) and in a rat model of chronic hypertension induced congestive heart failure (see above ¹⁸⁻²¹ and ²²Masumoto, A., Mohri, M., Shimokawa, H., Urakami, L., Usui, M., Takeshita, A., Suppression of coronary artery spasm by the rho-kinase inhibitor fasudil in patients with vasospastic angina. Circulation 105, 2002, 1545-1547; ²³Shimokawa, H., Iinuma, H., Kishida, H., et al., Antianginal effect of fasudil, a Rho-kinase inhibitor, in patients with stable effort angina: a multicenter study (abstract). Circulation 104[Suppl II], 2001, II691; ³⁶Morishige K, Shimokawa H, Eto Y, Kandabashi T, Miyata K, Matsumoto Y, Hoshijima M, Kaibuchi K, Takeshita A, Adenovirus-mediated transfer of dominant-negative rho-kinase induces a regression of coronary arteriosclerosis in pigs in vivo. Arterioscler Thromb Vasc Biol 21, 2001, 548-554; ³⁷Kandabashi T, Shimokawa H, Mukai Y, Matoba T, Kunihiro I, Morikawa K, Ito M, Takahashi S, Kaibuchi K, Takeshita A, Involvement of rho-kinase in agonists-induced contractions of arteriosclerotic human arteries. Arterioscler Thromb Vasc Biol 22, 2002, 243-248; ³⁸Liu M W, Roubin G S, King S B 3^{rd}, Restenosis after coronary angioplasty. Potential biologic determinants and role of intimal hyperplasia. Circulation 79, 1989, 1374-1387; ³⁹Shibata R, Kai H, Seki Y, Kato S, Morimatsu M, Kaibuchi K, Imaizumi T. Role of Rho-associated kinase in neointima formation after vascular injury. Circulation 103, 2001, 284-289).

Additionally, ROCKs can interact with other signalling pathways resulting in inhibition of phosphoinositide-3 kinase (PI-3K), endothelial nitric oxide synthase (eNOS) pathways, and activation of plasminogen activator inhibitor-1 (PAI-1) which may contribute to endothelial dysfunction like restenosis and atherosclerosis. Thus ROCK inhibitors have been suggested for the treatment of restenosis and atherosclerosis (see above ³⁶⁻³⁹ and Iwasaki, H. et al., High glucose induces plasminogen activator inhibitor-1 expression through Rho/Rho-kinase-mediated NF-kappaB activation in bovine aortic endothelial cells. Atherosclerosis, Jan. 31, 2007).

Vascular intimal thickening in vein grafts after surgery is the major cause of late graft failure. In a study with the ROCK inhibitor fasudil, the intimal thickening and vascular smooth muscle cell (VSMC) proliferation was significantly suppressed, whereas VSMC apoptosis was enhanced in the weeks following the procedure, suggesting that ROCK inhibitors can be used as a therapeutic agent for the prevention of graft failure^{36-39, 67}.

Injury to the adult vertebrate brain and spinal cord activates ROCKs, thereby causing neurodegeneration and inhibition of neuroregeneration like neurite growth and sprouting (⁵⁶Bito, H., Furuyashiki, T., Ishihara, H., Shibasaki, Y., Ohashi, K., Mizuno, K., Maekawa, M., Ishizaki, T., Narumiya, S., A critical role for a Rho-associated kinase, p160ROCK, in determining axon outgrowth in mammalian CNS neurons. Neuron 26, 2000, 431-441). Inhibition of ROCKs results in induction of new axonal growth, axonal rewiring across lesions within the CNS, accelerated regeneration and enhanced functional recovery after acute neuronal injury in mammals (spinal-cord injury, traumatic brain injury) (see above ⁶⁴ and ⁶⁰Hara, M. et al., Protein kinase inhibition by fasudil hydrochloride promotes neurological recovery after spinal cord injury in rats. J. Neurosurg. Spine 93, 94-101; ⁶¹Fournier, A. E., Takizawa, B. T. & Strittmatter, S. M., ROCK inhibition enhances axonal regeneration in the injured CNS. J. Neurosci. 23, 2003, 1416-1423; ⁶²Sung, J. K. et al., A possible role of RhoA/Rho-kinase in experimental spinal cord injury in rat. Brain Res. 959, 2003, 29-38; ⁶³Tanaka, H. et al., Cytoplasmic p21(Cip1/WAF1) enhances axonal regeneration and functional recovery after spinal cord injury in rats. Neuroscience 127, 2004, 155-164). ROCK inhibitors are therefore likely to be useful for regenerative (recovery) treatment of CNS disorders such as spinal cord injury, acute neuronal injury (stroke, traumatic brain injury) (⁵²Okamura N et al., Vasodilator effects of fasudil, a Rho-kinase inhibitor, on retinal arterioles in stroke-prone spontaneously hypertensive rats. J Ocul Pharmacol Ther. 23, 2007, 207-12; ⁵³Yagita Y et al., Rho-kinase activation in endothelial cells contributes to expansion of infarction after focal cerebral ischemia. J Neurosci Res. 85, 2007, 2460-9), Parkinson's disease, Alzheimer disease (⁵⁴Pedrini S et al., Modulation of statin-activated shedding of Alzheimer APP ectodomain by ROCK. PLoS Med.2, 2005, 18; ⁵⁵Burton A., NSAIDS and Alzheimer's disease: it's only Rock and Rho. Lancet Neurol. 3(1), 2004, 6) and other neurodegenerative disorders. Other neurodegenetarive disorders for which ROCK inhibitors are expected to be useful are Huntington's disease (Shao J, Welch W J, Diprospero N A, Diamond M I. Phosphorylation of profilin by ROCK1 regulates polyglutamine aggregation. Mol Cell Biol. September 2008;28(17):5196-208; Shao J, Welch W J, Diamond M I. ROCK and PRK-2 mediate the inhibitory effect of Y-27632 on polyglutamine aggregation. FEBS Lett. May 28, 2008;582(12): 1637-42), spinal muscular atrophy (Bowerman M, Shafey D, Kothary R., Smn depletion alters profilin II expression and leads to upregulation of the RhoA/ROCK pathway and defects in neuronal integrity. J Mol Neurosci. 2007-32(2):120-31) and amyotrophic lateral sclerosis. Inhibition of the Rho/ROCK pathway has also proved to be efficacious in other animal models of neurodegeneration like stroke^{52, 53} and in inflammatory and demyelinating diseases like multiple sclerosis (⁵¹Sun X et al., The selective Rho-kinase inhibitor Fasudil is protective and therapeutic in experimental autoimmune encephalomyelitis. J Neuroimmunol. 180, 2006, 126-34), acute and chronic pain (⁵⁷Inoue, M. et al., Initiation of neuropathic pain requires lysophosphatidic acid receptor signaling. Nature Med. 10, 2004, 712-718; ⁵⁸Ramer, L. M., Borisoff. J. F. & Ramer. M. S., Rho-kinase inhibition enhances axonal plasticity and attenuates cold hyperalgesia after dorsal rhizotomy. J Neurosci. 24, 2004, 10796-10805; ⁵⁹Tatsumi, S. et al., Involvement of Rho-kinase in inflammatory and neuropathic pain through phosphorylation of myristoylated alanine-rich C-kinase substrate (MARCKS). Neuroscience 131, 2005, 491-498).

ROCK inhibitors have been shown to possess anti-inflammatory properties by decreased cytokine release, e.g. TNFα. Thus they can be used as treatment for neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, osteoarthritis, osteoporosis, asthma, irritable bowel syndrome, or inflammatory bowel disease (⁷⁰Segain J. P., Rho kinase blockade prevents inflammation via nuclear factor kappa B inhibition: evidence in Crohn's disease and experimental colitis. Gastroenterology. 124(5), 2003, 1180-7). In addition, recent reports have demonstrated that inhibition of ROCK results in disruption of inflammatory cell chemotaxis as well as inhibition of smooth muscle contraction in models of pulmonary inflammation associated with asthma. Therefore, the inhibitors of the Rho/ROCK pathway should be useful for the treatment of asthma (see above ⁵¹ and ⁴⁷Kawaguchi A, Ohmori M, Harada K, Tsuruoka S, Sugimoto K, Fujimura A., The effect of a Rho kinase inhibitor Y-27632 on superoxide production, aggregation and adhesion inhuman polymorphonuclear leukocytes. Eur J Pharmacol 403, 2000, 203-208; ⁴⁸Lou Z, Billadeau D D, Savoy D N, Schoon R A, Leibson P.J., A role for a RhoA/ROCK/LIM-kinase pathway in the regulation of cytotoxic lymphocytes. J Immunol 167, 2001, 5749-5757; ⁴⁹Vicente-Manzanares M, Cabrero J R, Rey M, Perez-Martinez M, Ursa A, Itoh K, Sanchez-Madrid F., A role for the Rho-p160 Rho coiled-coil kinase axis in the chemokine stromal cell-derived factor-1alpha-induced lymphocyte actomyosinand microtubular organization and chemotaxis. J Immunol 168, 2002, 400-410; ⁵⁰Thorlacius K et al., Protective effect of fasudil, a Rho-kinase inhibitor, on chemokine expression, leukocyte recruitment, and hepatocellular apoptosis in septic liver injury. J Leukoc Biol. 79, 2006, 923-31).

Since ROCK inhibitors reduce cell proliferation and cell migration, they could be useful in treating cancer and tumor metastasis^{6,43-46}. ROCK inhibitors can also be beneficial in diseases with impaired blood brain barrier function, e.g. HIV-1 encephalitis (⁷¹Persidski Y et al., Rho-mediated regulation of tight junctions during monocyte migration across the blood-brain barrier in HIV-1 encephalitis (HIVE). Blood, 107, 2006, 4770-4780) and Alzheimer's disease (⁷²Man S-M et al., Peripheral T cells overexpress MIP-1a to enhance its transendothelial migration in Alzheimer's disease. Neurobiol. Of Aging 28, 2007, 485-496).

Furthermore, there is evidence suggesting that ROCK inhibitors suppress cytoskeletal rearrangement upon virus invasion, thus they also have potential therapeutic value in anti-viral and anti-bacterial applications (⁶⁹Favoreel H W, Cytoskeletal rearrangements and cell extensions induced by the US3 kinase of an alphaherpesvirus are associated with enhanced spread. Proc Natl Acad Sci USA, 102(25), 2006, 8990-5).

ROCKs have been reported to interfere with insulin signalling through serine phosphorylation of insulin receptor substrate-1 (IRS-1), in cultured VSMC. Activation of RhoA/ROCK was observed in skeletal muscles and aortic tissues of Zucker obese rats. Inhibition of ROCK, by fasudil for 4 weeks, reduced blood pressure, corrected glucose and lipid metabolism, improved insulin signalling and endothelial dysfunction. In another experiment administration of high dose fasudil completely suppressed the development of diabetes, obesity, and dyslipidemia and increased serum adiponectin levels in OLETF rats. ROCK inhibitors may therefore be useful for the treatment of insulin resistance and diabetes (see above ⁶⁷ and ⁶⁵Nakamura Y et al., Marked increase of insulin gene transcription by suppression of the Rho/Rho-kinase pathway. Biochem Biophys Res Commun. 350(1), 2006, 68-73; ⁶⁶Kikuchi Y et al., A Rho-kinase inhibitor, fasudil, prevents development of diabetes and nephropathy in insulin-resistant diabetic rats. J Endocrinol. 192(3), 2007. 595-603; ⁶⁸Goyo A et al., The Rho-kinase inhibitor, fasudil, attenuates diabetic nephropathy in streptozotocin-induced diabetic rats. Eur J Pharmacol. 568(1-3), 2007, 242-7).

The ROCK inhibitor Fasudil increased cerebral blood flow and was neuroprotective under CNS ischemic conditions. ROCK inhibitors are expected to be useful for the treatment of ischemic CNS disorders and may therefore improve functional outcome in patients suffering from stroke, vascular or AD type dementia^{52, 53}.

Due to the efficacy of Y-27632 and fasudil in animal models of epileptogenesis, ROCK inhibitors have been suggested for the use in the treatments of epilepsy and seizure disorders (Inan S Y, Büyükafsar K. Antiepileptic effects of two Rho-kinase inhibitors, Y-27632 and fasudil, in mice. Br. J. Pharmacol. advance online publication, 9 Jun. 2008; doi: 10.1038/bjp.2008.225)

ROCK inhibitors are also expected to be useful for the treatment of glaucoma^{34, 35}, psoriasis, retinopathy and benign prostatic hypertrophy.

Furthermore, there is evidence suggesting that ROCK inhibitors suppress cytoskeletal rearrangement upon virus invasion, thus they also have potential therapeutic value in anti-viral and anti-bacterial applications.

As ROCKs have been implicated in neuronal morphogenesis, connectivity, and plasticity in general, they are expected to be useful for the treatment of psychiatric disorders, e.g. depression, schizophrenia, obsessive compulsive disorder and bipolar disorders.

ROCK inhibitors have been described in e.g. WO 2007/026920, WO 2005/074643, and WO 2004/016597. However, their affinity and selectivity or their pharmacological profile is not yet satisfactory.

### SUMMARY

Provided generally herein are bicyclic compounds that are Rho kinases inhibitors, pharmaceutical compositions comprising such compounds, and methods for the treatment of disorders using these compounds and pharmaceutical compositions.

Presented herein are compounds of formula (I), or pharmaceutically acceptable salts, solvates, prodrugs, salts of prodrugs, or combinations thereof,

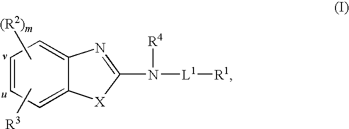

wherein

X is S or O;

R⁴ is hydrogen, alkyl, —(C₂₋₆ alkylene)-OR^{4g}, —(C₂₋₆ alkylene)-NR^{4k}R^{4m}, or haloalkyl;

L¹ is (CR^{p}R^{q})ₙ, (CR^{p}R^{q})ᵣ—X¹, or (CR^{p}R^{q})ᵣ—X¹—(CR^{p}R^{q})ₙ, wherein the (CR^{p}R^{q})ᵣ group of the (CR^{p}R^{q})ᵣ—X¹ and the (CR^{p}R^{q})ᵣ—X¹—(CR^{p}R^{q})ₙ is connected to N(R⁴) of formula (I),

X¹ is N(R⁵), O, or S; and

R¹ is —Si(R^{1a})₃, aryl, heteroaryl, heterocycle, cycloalkyl, or cycloalkenyl; each of the aryl, heteroaryl, heterocycle, cycloalkyl, and cycloalkenyl is optionally substituted with 1, 2, 3, 4, or 5 substituents as represented by R^{y};

or

L¹-R¹ together is formula (i)

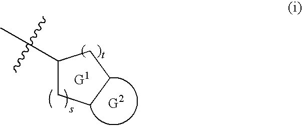

wherein s is 0, 1, 2, or 3;

t is 0, 1, 2, or 3; with the proviso that s and t are not both 0;

one or two CH₂ units of ring G¹ is optionally replaced by NH, O, N(O), S, S(O), or S(O)₂, ring G² is phenyl or monocyclic heteroaryl; and G¹ and G² are each independently unsubstituted or substituted with 1, 2, 3, 4, or 5 substituents as represented by R^{y′};

or
N(R⁴)-L¹-R¹ together is formula (ii)

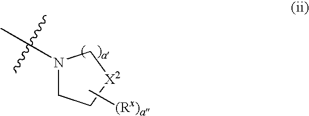

wherein

a′ is 1 or 2;

X² is CH₂, S, S(O), S(O)₂, O, or N(R⁶) wherein R⁶ is hydrogen, alkyl, —C(O)O(alkyl), —C(O)O(benzyl), or benzyl; provided that when X² is other than CH₂, then a′ is 2;

a″ is 0, 1 or2; and

each occurrence of R^{x} represents an optional substituent on any substitutable carbon or nitrogen atom of formula (ii), and is independently alkyl haloalkyl, aryl, arylalkyl, heteroaryl, or heteroarylalkyl; wherein each of the aryl and heteroaryl moieties, by itself or as part of the substituent, is independently unsubstituted or substituted with 1, 2, 3, or 4 R^{y} groups;

R^{1a}, at each occurrence, is independently alkyl, aryl, or arylalkyl wherein the aryl moiety, by itself of as part of the substituent, is independently unsubstituted or substituted with 1, 2, 3, 4, or 5 substituents selected from the group consisting of alkyl, haloalkyl, and halogen;

n is 1, 2, 3, 4, 5, or 6;

r is 2, 3, 4, 5, or 6;

R^{p} and R^{q}, at each occurrence, are each independently hydrogen, alkyl, haloalkyl, —OR^{gp, —NR}^{kp}R^{mp},—(C₁₋₆ alkylene)-OR^{gp}, —(C₁₋₆ alkylene)-NR^{kp}R^{mp}, or —(C₁₋₆ alkylene)-N—OR^{gp};

each R² represents an optional substituent on the benzene ring of formula (I), and at each occurrence, is independently selected from the group consisting of alkyl, alkenyl, alkynyl, halogen, NO₂, CN, haloalkyl, OR^{a}, —O—(C₂₋₆ alkylene)-NR^{k}R^{m}, OC(O)R^{a}, NR^{a}R^{b}, —N(R^{a})—(C₂₋₆ alkylene)-NR^{k}R^{m}, SR^{a}, S(O)R^{c}, S(O)₂R^{c}, S(O)₂NR^{a}R^{b}, C(O)R^{a}, C(O)OR^{a}, C(O)NR^{a}R^{b}, —(C₁₋₆ alkylene)-NO₂, —(C₁₋₆ alkylene)-CN, —(C₁₋₆ alkylene)-OR^{a}, —(C₁₋₆ alkylene)-OC(O)R^{a}, —(C₁₋₆ alkylene)-NR^{a}R^{b}, —(C₁₋₆ alkylene)-SR^{a}, —(C₁₋₆ alkylene)-S(O)R^{c}, —(C₁₋₆ alkylene)-S(O)₂R^{c}, —(C₁₋₆ alkylene)-S(O)₂NR^{a}R^{b}, —(C₁₋₆ alkylene)-C(O)R^{a}, —C₁₋₆ alkylene)-C(O)OR^{a}, and —(C₁₋₆ alkylene)-C(O)NR^{a}R^{b};

m is 0, 1, 2, or 3;

R³ is heterocycle or heteroaryl each of which is attaching to the benzene ring of formula (I) via position u or v, and is optionally substituted with 1, 2, 3, 4, or 5 substituents as represented by T, wherein each T is independently selected from the group consisting of alkyl, alkenyl, alkynyl, halogen, NO₂, CN, haloalkyl, OR^{d}, OC(O)R^{d}, NR^{d}R^{e}, SR^{d}, S(O)R^{f}, S(O)₂R^{f}, S(O)₂NR^{d}R^{c}, C(O)R^{d}, C(O)OR^{d}, C(O)NR^{d}R^{e}, —(C₁₋₆ alkylene)-NO₂, —(C₁₋₆ alkylene)-CN, —(C₁₋₆ alkylene)-OR^{d}, —(C₁₋₆ alkylene)-OC(O)R^{d}, —(C₁₋₆ alkylene)-NR^{d}R^{e}, —(C₁₋₆ alkylene)-SR^{d} (C₁₋₆ alkylene)-S(O)R^{f}, —(C₁₋₆ alkylene)-S(O)₂R^{f}, —(C₁₋₆ alkylene)-S(O)₂NR^{d}R^{e}, —(C₁₋₆ alkylene)-C(O)R^{d}, —(C₁₋₆ alkylene)-C(O)OR^{d}, and —(C₁₋₆ alkylene)-C(O)NR^{d}R^{e};

R^{y} and R^{y′}, at each occurrence, are each independently selected from the group consisting of alkyl, alkenyl, alkynyl, halogen, NO₂, CN, oxo, haloalkyl, OR^{g}, —O—(C₁₋₆ alkylene)-Si(R^{1a})₃, —O—(C₂₋₆ alkylene)-NR^{k}R^{m}, —OC(O)R^{g}, —NR^{k}R^{m}, —N(R^{g})—(C₂₋₆ alkylene)-NR^{k}R^{m}, —N(R^{g})S(O)₂R^{j}, —N(R^{g})C(O)OR^{g}, —SR^{g}, —S(O)R^{j}, —S(O)₂R^{j}, —S(O)₂NR^{k}R^{m}, —C(O)R^{g}, —C(O)OR^{g}, —C(O)NR^{k}R^{m}, —C(O)N(R^{g})(C₂₋₆ alkylene-NR^{k}R^{m}), —C(O)N(R^{g})(C₂₋₆ alkylene-OR^{g}), —(C₁₋₆ alkylene)-NO₂, —(C₁₋₆ alkylene)-CN, —(C₁₋₆ alkylene)-OR^{g}, —(C₁₋₆ alkylene)-OC(O)R^{g}, —(C₁₋₆ alkylene)-NR^{k}R^{m}, —(C₁₋₆ alkylene)-SR^{g}, —(C₁₆ alkylene)-S(O)R^{j}, —(C₁₋₆ alkylene)-S(O)₂R^{j}, —(C₁₋₆ alkylene)-S(O)₂NR^{k}R^{m}, —(C₁₋₆ alkylene)-C(O)R^{g}, —(C₁₋₆ alkylene)-C(O)OR^{g}, and —(C₁₋₆ alkylene)-C(O)NR^{k}R^{m};

w is 1, 2, or 3;

R^{a}, R^{b}, R^{d}, R^{e}, R^{g}, R^{4g}, and R^{gp}, at each occurrence, are each independently hydrogen, alkyl, or haloalkyl;

R^{c}, R^{f} and R^{j}, at each occurrence, are each independently alkyl or haloalkyl;

R^{k}, R^{m}, R^{4k}, R^{4m}, R^{kp}, and R^{mp}, at each occurrence, are each independently hydrogen, alkyl, or haloalkyl;

R^{k} and R^{m}, R^{4k} and R^{4m}, R^{kp} and R^{mp}, together with the nitrogen atom to which they are attached, optionally form a 5- or 6-membered monocyclic heterocycle, said monocyclic heterocycle optionally contains 0 or 1 additional heteroatom, and is optionally substituted with 1, 2, 3, or 4 substituents independently selected from the group consisting of oxo, alkyl, and haloalkyl; and

R⁵ is hydrogen, alkyl, haloalkyl, alkoxyalkyl, haloalkoxyalkyl, or hydroxyalkyl;

with the proviso that when X is O then R³ is not pyrimidin-5-yl; and with the further proviso that when X is S, L¹ is (CR^{p}R^{q})ₙ wherein n is 1, R^{p} and R^{q} are hydrogen, and R¹ is optionally substituted phenyl, then R³ is not imidazolyl.

Another aspect relates to pharmaceutical compositions comprising therapeutically effective amounts of one or more compounds presented herein, or pharmaceutically acceptable salts thereof, in combination with one or more pharmaceutically acceptable carrier, adjuvants, excipients, or other auxiliary substances. Said pharmaceutical compositions are useful for treating diseases described herein.

Compounds described herein are useful for the prevention or treatment of diseases associated with abnormal ROCK activity. Thus, pharmaceutically effective compositions of such compounds or pharmaceutically acceptable salts thereof are useful for the prevention or treatment of said diseases.

The present compounds have inhibitory activity against ROCK-1, and ROCK-2 kinases and are thus useful for the inhibition of such kinases. Accordingly, the present compounds or pharmaceutically acceptable salts thereof may be useful as active ingredients for the preparation of compositions, which enable preventive and/or therapeutic treatment of diseases or conditions caused by abnormal ROCK kinases (including ROCK-1 and ROCK-2) activity. The diseases which respond to the modulation of ROCKs, in particular to ROCKs inhibition include, but are not limited to, cardiovascular diseases such as hypertension, chronic and congestive heart failure, cardiac hypertrophy, restenosis, chronic renal failure, atherosclerosis, asthma, male erectile dysfunctions, female sexual dysfunction, over-active bladder syndrome, neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, irritable bowel syndrome, or inflammatory bowel disease. In addition, based on their neurite outgrowth inducing effects, ROCK inhibitors can be used as drugs for neuronal regeneration, inducing new axonal growth and axonal rewiring across lesions within the CNS. ROCK inhibitors are therefore useful for regenerative (recovery) treatment of CNS disorders such as spinal cord injury, acute neuronal injury (stroke, traumatic brain injury). Parkinson's disease, Alzheimer disease and other neurodegenerative disorders, such as, in particular, Huntington's disease, spinal muscular atrophy, and amyotrophic lateral sclerosis. Since ROCK inhibitors reduce cell proliferation and cell migration, they could be useful in treating cancer and tumor metastasis. Furthermore, there is evidence suggesting that ROCK inhibitors suppress cytoskeletal rearrangement upon virus invasion, thus they also have potential therapeutic value in anti-viral and anti-bacterial applications. ROCK inhibitors may also be useful for the treatment of insulin resistance and diabetes. ROCK inhibitors may furthermore be useful for the treatment of ischemic CNS disorders, vascular or AD type dementia, glaucoma, psoriasis, retinopathy, benign prostatic hypertrophy, psychiatric disorders, in particular depression, schizophrenia, obsessive compulsive disorder and bipolar disorder, epilepsy and seizure disorders, for decreasing ischemia-reperfusion injury, myocardial infarct size and myocardial fibrosis, and for the prevention of graft failure. Accordingly, the compounds described herein can be used for treating the above-listed disorders. More preferably, they are used for treating pain, asthma, Alzheimer's disease, multiple sclerosis, rheumatoid arthritis and spinal cord injuries.

A further aspect herein provides methods of treating diseases as described herein above. Said methods comprise administering to the subject (including human) in need thereof therapeutically effective amounts of one or more compounds described herein or pharmaceutically acceptable salts thereof, with or without one or more pharmaceutically acceptable carriers, excipients, adjuvants, or other auxiliary substances.

Further, the present application provides the use of compounds described herein or pharmaceutically acceptable salts thereof, with or without one or more pharmaceutically acceptable carriers, excipients, adjuvants, or other auxiliary substances, in the manufacture of medicaments for the treatment of the diseases or conditions described herein.

These and other objects are described in the following paragraphs. These objects should not be deemed to narrow the scope of the invention.

### DETAILED DESCRIPTION

Compounds of formula (I) are disclosed

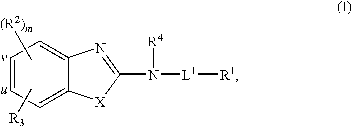

wherein X, R¹, R², R³, R⁴, m, and L¹ are as defined above in the Summary and below in the Detailed Description. Compositions comprising such compounds and methods for treating conditions and disorders using such compounds and compositions are also disclosed.

In various embodiments, the present application provides at least one variable that occurs more than one time in any substituent or in the compounds or any other formulae herein. Definition of a variable on each occurrence is independent of its definition at another occurrence. Further, combinations of variables or substituents are permissible only if such combinations result in stable compounds. Stable compounds are compounds that can be isolated from a reaction mixture.

### a. DEFINITIONS

As used in the specification and the appended claims, unless specified to the contrary, the following terms have the meaning indicated:

The term “alkenyl” as used herein, means a straight or branched hydrocarbon chain containing, for example, from 2 to 10 carbons, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” denotes a divalent group derived from a straight or branched hydrocarbon chain of 2, 3, or 4 carbon atoms and contains at least one carbon-carbon double. Representative examples of alkenylene include, but are not limited to, —CH═CH— and —CH₂CH═CH—.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” as used herein, means an alkoxy group, as defined herein, appended to the parent molecular moiety through an alkylene group, as defined herein. Representative examples of alkoxyalkyl include, but are not limited to, tert-butoxymethyl, 2-ethoxyethyl, 2-methoxyethyl, and methoxymethyl.

The term “alkyl” as used herein, means a saturated, straight or branched hydrocarbon chain containing, for example, from 1 to 10 carbon atoms. The term “C₁₋₆ alkyl” as used herein, means a saturated, straight or branched hydrocarbon chain containing from 1 to 6 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 1-methylbutyl, 2-methylbutyl, 3-methylbutyl, 1,1-dimethylpropyl, 1,2-dimethylpropyl, 2,2-dimethylpropyl, 1-methylpropyl, 1-ethylpropyl, 1,2,2-trimethylpropyl, 3-methylhexyl, 2,2-dimethylpentyl 2,3-dimethylpentyl, n-heptyl, n-octyl, n-nonyl, and n-decyl.

The term “alkylene” means a divalent group derived from a saturated, straight or branched hydrocarbon chain containing, for example, from 1 to 10 carbon atoms. The term “C₂₋₆-alkylene” means an alkylene group having from 2 to 6 carbon atoms. The term “C₁₋₆ alkylene” means those alkylene groups having from 1 to 6 carbon atoms. Representative examples of alkylene, C₂₋₆ alkylene, and C₁₋₆ alkylene include, for example, —CH₂—, —CH(CH₃)—, —CH(C₂H₅), —CH(CH(CH₃)(C₂H₅))—, —C(H)(CH₃)CH₂CH₂—, —C(CH₃)₂—, —CH₂CH₂—, —CH₂CH₂CH₂—, —CH₂CH₂CH₂CH₂—, and —CH₂CH(CH₃)CH₂—.

The term “alkynyl” as used herein, means a straight or branched hydrocarbon group chain containing from, for example, 2 to 10 carbon atoms and containing at least one carbon-carbon triple bond. Representative examples of alkynyl include, but are not limited, to acetylenyl, 1-propynyl, 2-propynyl, 1,1-dimethylprop-2-ynyl, 1-propyl-pent-3-ynyl, 3-butynyl, 2-pentynyl, and 1-butynyl.

The term “aryl,” as used herein, means optionally substituted phenyl, a bicyclic aryl, or a tricyclic aryl. The bicyclic aryl is naphthyl (including, but not limited thereto, naphth-1-yl, naphth-2-yl), or a phenyl fused to a monocyclic cycloalkyl, or a phenyl fused to a monocyclic cycloalkenyl. Representative examples of the bicyclic aryl include, but are not limited to, dihydroindenyl, indenyl, naphthyl (including, but not limited thereto, naphth-1-yl, naphth-2-yl), dihydronaphthalenyl, and tetrahydronaphthalenyl. The tricyclic aryl is exemplified by a bicyclic aryl fused to a monocyclic cycloalkyl, or a bicyclic aryl fused to a monocyclic cycloalkenyl, or a bicyclic aryl fused to a phenyl. Representative examples of tricyclic aryls include, but are not limited to, anthracene, phenanthrene, dihydroanthracenyl, fluorenyl, 1,2-dihydroacenaphthylenyl, and tetrahydrophenanthrenyl. The phenyl, bicyclic, and tricyclic aryls are attached to the parent molecular moiety through any carbon atom contained within the phenyl, bicyclic, and tricyclic aryls respectively.

The term “arylalkyl,” as used herein, means an aryl group, as defined herein, appended to the parent molecular moiety through an alkylene group, as defined herein.

The term “cycloalkenyl” as used herein, means a monocyclic or bicyclic ring system containing zero heteroatoms in the ring, each of which is optionally substituted. The monocyclic cycloalkenyl has three-, four-, five-, six-, seven- or eight carbon atoms and zero heteroatoms. The three or four-membered ring systems have one double bond, the five-or six-membered ring systems have one or two double bonds, and the seven- or eight-membered ring systems have one, two or three double bonds. Representative examples of monocyclic cycloalkenyls include, but are not limited to, 2-cyclohexen-1-yl, 3-cyclohexen-1-yl, 2,4-cyclohexadien-1-yl and 3-cyclopenten-1-yl. Bicyclic cycloalkenyls are exemplified by a monocyclic cycloalkenyl fused to a monocyclic cycloalkyl, or a monocyclic cycloalkenyl fused to a monocyclic cycloalkenyl. Representative examples of bicyclic ring systems include, but are not limited to 3a,4,5,6,7,7a-hexahydro-1H-indenyl, 4,5,6,7-tetrahydro-3aH-indene, and octahydronaphthalenyl. The cycloalkenyl groups of present compunds are appended to the parent molecular moiety through any substitutable carbon atom within the groups, and may contain one or two alkylene bridges of 1, 2, 3, or 4 carbon atoms, wherein each bridge links two non-adjacent atoms within the groups.

The term “cycloalkyl” as used herein, means a monocyclic or a bicyclic cycloalkyl, or a spirocyclic cycloalkyl. The monocyclic cycloalkyl is a carbocyclic ring system containing 3, 4, 5, 6, 7, or 8 carbon atoms and zero heteroatoms as ring atoms, and zero double bonds. Examples of monocyclic cycloalkyls include cyclopropyl, cyclobutyl, cyclopentyl, cyclohexyl, cycloheptyl, and cyclooctyl. Bicyclic cycloalkyl is exemplified by a monocyclic cycloalkyl fused to a monocyclic cycloalkyl. Examples of bicyclic cycloalkyls include, but are not limited to, bicyclo[4.1.0]heptane, bicyclo[6.1.0]nonane, octahydroindene, and decahydronaphthalene. The monocyclic and the bicyclic cycloalkyl groups may contain one or two alkylene bridges of 1, 2, 3, or 4 carbon atoms, wherein each bridge links two non-adjacent atoms within the groups. Examples of such bridged cycloalkyls include, but are not limited to, bicyclo[2.2.1]heptane, bicyclo[3.1.1]heptane, bicyclo[2.2.2]octane, bicyclo[3.3.1]nonane, adamantane (tricyclo[3.3.1.1^{3,7}]decane), and noradamantane (octahydro-2,5-methanopentalene). Spirocyclic cycloalkyl is exemplified by a monocyclic or a bicyclic cycloalkyl, wherein two of the substituents on the same carbon atom of the ring, together with said carbon atom, form a 4-, 5-, or 6-membered monocyclic cycloalkyl. Example of a spirocyclic cycloalkyl includes, but is not limited to, spiro[2.5]octane. The monocyclic, bicyclic, and spirocyclic cycloalkyl groups of the present compounds can be appended to the parent molecular moiety through any substitutable carbon atom of the groups.

The term “halo” or “halogen” as used herein, means —Cl, —Br, 13 I, or —F.

The term “haloalkyl” as used herein, means an alkyl group, as defined herein, in which one, two, three, four, five, six, or seven hydrogen atoms are replaced by halogen. Representative examples of haloalkyl include, but are not limited to, chloromethyl, difluoromethyl, 2-fluoroethyl, 2,2-difluoroethyl, trifluoromethyl, 2,2,2-trifluoroethyl, 2,2,2-trifluoro-1,1-dimethylethyl, difluoromethyl, 3,3,3-trifluoropropyl, pentafluoroethyl, 2-chloro-3-fluoropentyl, and 2-iodoethyl.

The term “haloalkoxy” as used herein, means an alkoxy group, as defined herein, in which one, two, three, four, five, six, or seven hydrogen atoms are replaced by halogen. Representative examples of haloalkoxy include, but are not limited to, chloromethoxy, difluoromethoxy, 2-fluoroethoxy, 2,2-difluoroethoxy, and trifluoromethoxy.

The term “haloalkoxyalkyl” as used herein, means at least one haloalkoxy group is appended to the parent molecular moiety through an alkylene group, as defined herein. Representative examples of haloalkoxyalkyl include, but are not limited to, difluoromethoxymethyl and trifluoromethoxymethyl.

The term “heteroatom,” as used herein, means an oxygen, sulfur or nitrogen atom.

The term “heteroaryl,” as used herein, means a monocyclic heteroaryl or a bicyclic heteroaryl. The monocyclic heteroaryl is a 5- or 6-membered ring containing at least one heteroatom independently selected from the group consisting of O, N, and S, where the nitrogen and sulfur heteroatoms may optionally be oxidized and the nitrogen atoms may optionally be quaternized. The 5-membered ring contains two double bonds and one, two, three, or four heteroatoms. The 6-membered ring contains three double bonds and one, two, three, or four heteroatoms. Examples of monocyclic heteroaryl include, but are not limited to, furanyl, imidazolyl, isoxazolyl, isothiazolyl, oxadiazolyl, oxazolyl, pyridinyl (including, but not limited thereto, pyridin-4-yl), pyridazinyl, pyrimidinyl (including, but not limited thereto, pyrimidin-4-yl), pyrazinyl, pyrazolyl (including, but not limited thereto, 1H-pyrazol-5-yl), pyrrolyl, tetrazolyl, thiadiazolyl, thiazolyl (including, but not limited thereto, 1,3-thiazol-4-yl), thienyl (including, but not limited thereto, thien-2-yl), and triazolyl. The bicyclic heteroaryl is exemplified by a monocyclic heteroaryl fused to phenyl, or a monocyclic heteroaryl fused to a monocyclic cycloalkyl, or a monocyclic heteroaryl fused to a monocyclic cycloalkenyl, or a monocyclic heteroaryl fused to a monocyclic heteroaryl, or a monocyclic heteroaryl fused to a monocyclic heterocycle. Representative examples of bicyclic heteroaryls include, but are not limited to, benzofuranyl, benzoxadiazolyl, 1,3-benzothiazolyl, benzimidazolyl, benzodioxolyl, benzothienyl, 1H-pyrrolo[2,3-b]pyridinyl (including, but not limited thereto, 1H-pyrrolo[2,3-b]pyridin-4-yl), 7H-pyrrolo[2,3-d]pyrimidinyl (including, but not limited thereto, 7H-pyrrolo[2,3-d]pyrimidin-4-yl), chromenyl, cinnolinyl, indolyl, indazolyl (including, but not limited thereto, 1H-indazol-5-yl), isoindolyl, isoquinolinyl, naphthyridinyl, quinolinyl, and thienopyridinyl. The monocyclic and the bicyclic heteroaryl groups are optionally substituted and are connected to the parent molecular moiety through any substitutable carbon atom or any substitutable nitrogen atom contained within the groups.

The term “heteroarylalkyl,” as used herein, means a heteroaryl group as defined herein, appended to the parent molecular moiety through an alkylene group, as defined herein.

The term “heterocycle” or “heterocyclic” as used herein, means a monocyclic, bicyclic, or a spirocyclic ring system containing at least one heteroatom selected from nitrogen atom, oxygen atom, and/or sulfur atoms, where the nitrogen and sulfur heteroatoms may optionally be oxidized and the nitrogen atoms may optionally be quarternized. The monocyclic heterocycle is a 3-, 4-, 5-, 6-, 7-, or 8-membered monocyclic ring containing at least one heteroatom independently selected from the group consisting of O, N, and S. The 3- or 4-membered ring contains 1 heteroatom selected from the group consisting of O, N and S, and optionally one double bond. The 5-membered ring contains zero or one double bond, and one, two or three heteroatoms in the ring selected from the group consisting of O, N and S. The 6-, 7-, or 8-membered ring contains zero, one, or two double bonds, and one, two, or three heteroatoms in the ring selected from the group consisting of O, N and S. Representative examples of monocyclic heterocycles include, but are not limited to, azetidinyl, azepanyl, aziridinyl, diazepanyl, 1,3-dioxanyl, 1,4-dioxanyl, 1,3-dioxolanyl, 4,5-dihydroisoxazol-5-yl, 3,4-dihydropyran-6-yl, 1,3-dithiolanyl, 1,3-dithianyl, imidazolinyl, imidazolidinyl, isothiazolinyl, isothiazolidinyl, isoxazolinyl, isoxazolidinyl, morpholinyl (including, but not limited thereto, morpholin-4-yl), oxadiazolinyl, oxadiazolidinyl, oxazolinyl, oxazolidinyl, oxetanyl, piperazinyl (including, but not limited thereto, piperazin-1-yl), piperidinyl (including, but not limited thereto, piperidin-1-yl), pyranyl, pyrazolinyl, pyrazolidinyl, pyrrolinyl, pyrrolidinyl (including, but not limited thereto, pyrrolidin-1-yl), tetrahydrofuranyl, tetrahydropyranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is exemplified by a monocyclic heterocycle fused to a phenyl group, or a monocyclic heterocycle fused to a monocyclic cycloalkylgroup, or a monocyclic heterocycle fused to a monocyclic cycloalkenyl group, or a monocyclic heterocycle fused to a monocyclic heterocycle group. Examples of bicyclic heterocycle include, but are not limited to, dihydro-1,5-benzodioxepinyl (including, but not limited thereto, 3,4-dihydro-2H-1,5-benzodioxepin-6-yl), benzodioxolyl (including, but not limited thereto, 1,3-benzodioxol-5-yl), 1,3-benzodithiolyl, 2,3-dihydro-1,4-benzodioxinyl (including, but not limited thereto, 2,3-dihydro-1,4-benzodioxin-5-yl), dihydrobenzofuranyl (including, but not limited thereto, 2,3-dihydro-1-benzofuran-7-yl), 2,3-dihydro-1-benzothienyl, 2,3-dihydro-1H-indolyl, dihydroisochromenyl (including 3,4-dihydro-1H-isochromen-3-yl), and 1,2,3,4-tetrahydroquinolinyl. Spirocyclic heterocycle means a monocyclic or bicyclic heterocycle ring wherein two substituents on the same carbon atom, together with said carbon atom, form a 4-, 5-, or 6-membered monocyclic cycloalkyl. An example of a spiroheterocycle includes, but is not limited to, 5-oxaspiro[3,4]octane. The heterocycle groups are connected to the parent molecular moiety through any substitutable carbon atom or any substitutable nitrogen atom contained within the group. The monocyclic or bicyclic heterocycle groups may contain an alkenylene bridge of 2, 3, or 4 carbon atoms, or one or two alkylene bridges of 1, 2, 3, or 4 carbon atoms, wherein each bridge links two non-adjacent carbon atoms within the groups. Examples of such bridged heterocycles include, but are not limited to, oxaadamantane (2-oxatricyclo[3.3.1.1^{3,7}]decane), octahydro-2,5-epoxypentalene, hexahydro-2H-2,5-methanocyclopenta[b]furan, hexahydro-1H-1,4-methanocyclopenta[c]furan, oxabicyclo[2.2.1]heptane and 2,4-dioxabicyclo[4.2.1]nonane.

Any oxidized form of nitrogen or sulfur, and the quarternized form of any basic nitrogen in the heterocycle or heteroaryl groups are also contemplated.

The term “hydroxyalkyl” as used herein, means at least one OH group is appended to the parent molecular moiety through a C₂₋₆ alkylene group, as defined herein. Representative examples of hydroxyalkyl include, but are not limited to, 2-hydroxyethyl, 3-hydroxypropyl, 2,3-dihydroxypropyl, 2,3-dihydroxypentyl, and 2-ethyl-4-hydroxyheptyl.

The term “oxo” means ═O.

The symbol

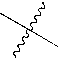

means the point of attachment to the parent moiety.

### b. COMPOUNDS

Present compounds have the formula (I) as described above.

Particular values of variable groups in formulae herein are as follows. Such values may be used where appropriate with any of the other values, definitions, claims or embodiments defined hereinbefore or hereinafter.

As described generally in the Summary section for compounds of formula (I), R³ is a heteroaryl or heterocycle attaching to the benzene ring of formula (I) at position u or v. In certain embodiments, R³ is attached to position u of formula (I). Thus certain embodiments are directed to compounds of formula (Ia)

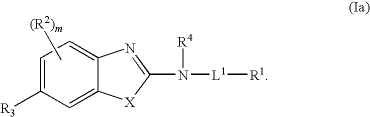

In other embodiments, R³ is attached to position v of formula (I). Thus, certain embodiments are directed to compounds of formula (Ib)

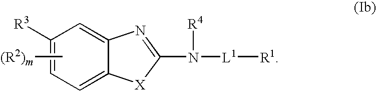

X, R¹, R², R³, R⁴, m, and L¹ of formula (Ia) and (Ib) have the meanings as described in the Summary and the Detailed Description sections for formula (I).

R³ has values as generally described in the Summary section. Examples of present compounds include, but are not limited to those wherein R³, for example, is pyridinyl, pyrimidinyl, pyrazolyl, pyridazinyl, indazolyl, 1H-pyrrolo[2,3-b]pyridinyl, 7H-pyrrolo[2,3-d]pyrimidinyl, and morpholinyl, each of which is optionally substituted as described in the Summary and embodiments herein.

In some embodiments of the present compounds, R³ is optionally substituted pyridinyl (including, but not limited thereto, optionally substituted pyridin-4-yl).

In other embodiments, R³ is, for example, pyrimidinyl (including, but not limited thereto, pyrimidin-4-yl), pyrazolyl (including, but not limited thereto, 1H-pyrazol-5-yl), indazolyl (including, but not limited thereto, 1H-indazol-5-yl), 1H-pyrrolo[2,3-b]pyridinyl (including, but not limited thereto, 1H-pyrrolo[2,3-b]pyridin-4-yl), and 7H-pyrrolo[2,3-d]pyrimidinyl (including, but not limited thereto, 7H-pyrrolo[2,3-d]pyrimidin-4-yl), each of which is optionally substituted.

Each R³ is optionally substituted with 1, 2, 3, 4, or 5 substituents as represented by T wherein T is as described in the Summary. In conjunction with any of the above or below embodiments, each occurrence of T is independently, for example, alkyl such as C₁₋₆ alkyl (e.g. methyl, ethyl, and the like), halogen (e.g. fluorine, chlorine, and the like), or —C₁₋₆ alkylene)-NR^{d}R^{e} wherein R^{d} and R^{e} are as described in the Summary (for example, in certain embodiments. R^{d} and R^{e} are each independently hydrogen or C₁₋₆ alkyl (e.g. methyl)).

As generally described in the Summary section. X is S or O; provided that when X is O, then R³ is not pyrimidin-5-yl.

Certain embodiments are directed to compounds wherein X is S.

Certain embodiments are directed to compounds wherein X is O.

L¹ has values as generally described in the Summary. In certain embodiments, L¹ is (CR^{p}R^{q})ₙ or (CR^{p}R^{q})ᵣ—X¹.

In some embodiments of compounds of formula (I), (Ia), or (Ib), L¹ is (CR^{p}R^{q})ₙ.

In some embodiments of compounds of formula (I), (Ia), or (Ib), L¹ is (CR^{p}R^{q})ᵣ—X¹.

R^{p}, R^{q}, r, and n are as described in the Summary section. In conjunction with any above or below embodiments, n, for example, is 1, 2, or 3. In certain embodiments, n is 1 or 2,R^{p}, at each occurrence is, for example, independently hydrogen or C₁₋₆ alkyl (for example, methyl, ethyl), and R^{q}, at each occurrence, is independently hydrogen, C₁₋₆ alkyl (for example, methyl, ethyl), —OR^{gp} (e.g. OH), —(C₁₋₆ alkylene)-OR^{gp} (e.g. —CH₂OH, —(CH₂)₂OH), or —(C₁₋₆ alkylene)-N—OR^{gp} (e.g. —CH₂)₂—NOH). In certain embodiments, R^{p} and R^{q}, at each occurrence, are each independently hydrogen or C₁₋₆ alkyl (for example, methyl, ethyl, and the like). In conjunction with any above or below embodiments, X¹, for example is O. In certain examples of compounds of formula (I), (Ia), and (Ib), r is 2.

R¹ has values as described generally in the Summary and in embodiments herein.

In some embodiments of the compounds of formula (I), (Ia), and (Ib), examples of R¹ include, but are not limited to,

—Si(R^{1a})₃ (e.g. —Si(C₁₋₆ alkyl)₃ wherein each C₁₋₆ alkyl is the same or different);

aryl such as, but not limited to, phenyl and naphthyl (including, but not limited thereto, naphth-1-yl, naphth-2-yl), and

heterocycle such as, but not limited to, dihydro-1,5-benzodioxepinyl (including, but not limited thereto, 3,4-dihydro-2H-1,5-benzodioxepin-6-yl), benzodioxolyl (including, but not limited thereto, 1,3-benzodioxol-5-yl), 2,3-dihydro-1,4-benzodioxinyl (including, but not limited thereto, 2,3-dihydro-1,4-benzodioxin-5-yl), dihydrobenzofuranyl (including, but not limited thereto, 2,3-dihydro-1-benzofuran-7-yl), and dihydroisochromenyl (including 3,4-dihydro-1H-isochromen-3-yl).

In certain embodiments, R¹ is optionally substituted phenyl.

Each R¹ is optionally substituted with 1, 2, 3, 4, or 5 substituents as represtented by R^{y} as described in the Summary. In conjunction with any of the above or below embodiments, each R^{y} is independently, for example, but not limited to, C₁₋₆ alkyl (for example, methyl, ethyl), halogen (for example, fluorine, chlorine), haloalkyl (for example, trifluoromethyl), OR^{g}, —O—(C₂₋₆ alkylene)-NR^{k}R^{m}, —NR^{k}R^{m}, —N(R^{g})S(O)₂R^{j}, —SR^{g}, —C(O)OR^{g}), —C(O)N(R^{g})(C₂₋₆ alkylene-NR^{k}R^{m}), —C(O)N(R^{g})(C₂₋₆ alkylene-OR^{g}), or —(C₁₋₆ alkylene)-NR^{k}R^{m}.

The variables R^{g}, R^{k}, R^{m}, and R^{j} for R¹ group are as disclosed in the Summary and in embodiments herein. In certain embodiments, R^{g}, for example, is hydrogen, C₁₋₆ alkyl (such as, but not limited to, methyl, ethyl, isopropyl, and propyl), or haloalkyl (such as, but not limited to, trifluoromethyl and difluoromethyl). R^{j}, for example, is C₁₋₆ alkyl such as, but not limited to, methyl. R^{k} and R^{m}, at each occurrence, are each independently, hydrogen or C₁₋₆ alkyl such as, but not limited to, methyl. R^{k} and R^{m}, together with the nitrogen atom to which they are attached, optionally form a 5- or 6-membered monocyclic heterocycle as described in the Summary. Examples of said monocyclic heterocycle include, but are not limited to, piperazinyl, piperidinyl, pyrrolidinyl, and morpholinyl, each of which is optionally substituted as described in the Summary.

In some embodiments of the compounds of formula (I), (Ia), and (Ib), L¹-R¹ together is formula (i) as described generally in the Summary section

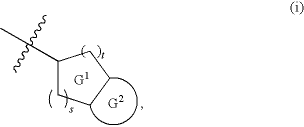

wherein s, t, G¹, and G² are as described generally in the Summary and in embodiments herein.

In certain embodiments, s is 2, t is 0, and G² is phenyl. It is thus appreciated that compounds of formula (Ic)

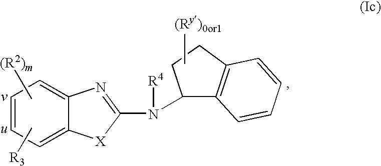

wherein R^{y}′, R², R³, R⁴, m, and X are as described generally in the Summary and in embodiments herein are also contemplated. In certain embodiments, an example of R^{y′}, if present, includes, but not limited to OR^{g} wherein R^{g} is as described in the Summary and can be, for example, hydrogen.

R⁴ has values as described generally in the Summary. In conjunction with any of the above or below embodiments, R⁴ of compounds having formula (I), (Ia), (Ib), and (Ic) is, for example, hydrogen, C₁₋₆ alkyl (e.g. methyl and the like), —(C₂₋₆ alkylene)-OR^{4g}, —(C₂₋₆ alkylene)-NR^{4k}R^{4m}, or haloalkyl, wherein R^{4g}, R^{4k}, and R^{4m} are as described generally in the Summary and in embodiments herein. For example, R^{4g}, R^{4k}, and R^{4m} are each independently hydrogen or C₁₋₆ alkyl (e.g. methyl, ethyl, and the like). R^{4k} and R^{4m}, together with the nitrogen atom to which they are attached, optionally form a monocyclic heterocycle (e.g. optionally substituted pyrrolidinyl or optionally substituted piperidinyl) as described in the Summary. In conjunction with any of the above or below embodiments, R⁴, for example, is hydrogen, methyl, ethyl, —(CH₂)₂—OH, —(CH₂)₂—N(CH₃)₂, —(CH₂)₂-(pyrrolidin-1-yl), or —(CH₂)₂-(piperidin-1-yl), wherein the pyrrolidin-1-yl and the piperidin-1-yl are optionally substituted as described in the Summary. In certain embodiments, R⁴ is hydrogen or methyl. In yet other embodiments, R⁴ is hydrogen.

In some embodiments of the compounds of formula (I), (Ia), and (Ib), N(R⁴)-L¹-R¹ together is formula (ii) as described generally in the Summary section

Thus, included in the present application are compounds of formula (Id)

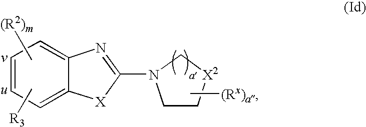

or pharmaceutically acceptable salts, solvates, prodrugs, salts of prodrugs, or any combination thereof, wherein X, X², R², R³, R^{x}, m, a′, and a″ are as described in the Summary and in embodiments herein.

In certain embodiments, a′ is 1, X₂ is CH₂, and a″ is 0 or 1. R^{x}, if present, is as described generally in the Summary and in embodiments herein. For example, in conjunction with any of the above or below embodiments, R^{x} is aryl (such as, but not limited to, phenyl); arylalkyl (such as, but not limited to, benzyl), or heteroaryl (such as, but not limited to, thienyl, 1,3-thiazolyl), wherein the aryl and heteroaryl moieties are each optionally substituted as described in the Summary, for example, substituted with 1, 2, 3, or 4 substituents selected from C₁₋₆ alkyl (such as, but not limited to, methyl, ethyl); halogen (such ash but not limited to, fluorine, chlorine); OR^{g}, —O—(C₂₋₆ alkylene)-NR^{k}R^{m}; wherein R^{g}, R^{k}, and R^{m} are as described generally in the Summary and in embodiments herein. In certain embodiments, R^{g}, R^{k}, and R^{m} are each independently hydrogen or C₁₋₆ alkyl (e.g. methyl), R^{k} and R^{m}, together with the nitrogen atom to which they are attached optionally form monocyclic heterocycle as described in the Summary, for example, an optionally substituted piperidinyl.

R² and m for formula (I), (Ia), (Ib), (Ic), and (Id) are as described generally in the Summary. In certain embodiments, m is 0.

It is appreciated that compounds of formula (I), (Ia), (Ib), (Ic), and (Id), with combinations of the above embodiments, including particular, more particular and preferred embodiments are contemplated.

Accordingly, one aspect of the present application provides a group of compounds of formula (I), (Ia), and (Ib) wherein X is S and L¹ is (CR^{p}R^{q})ₙ or (CR^{p}R^{q})ᵣ—X¹.

Another aspect is related to a group of compounds of formula (I), (Ia), and (Ib) wherein X is S and L¹ is (CR^{p}R^{q})ₙ.

Yet another aspect provides a group of compounds of formula (I), (Ia), and (Ib) wherein X is S and L¹ is (CR^{p}R^{q})ᵣ—X¹.

Another aspect of the present application provides a group of compounds of formula (I), (Ia), and (Ib) wherein X is O and L¹ is (CR^{p}R^{q}), or (CR^{p}R^{q})ᵣ—X¹.

Another aspect is related to a group of compounds of formula (I), (Ia), and (Ib) wherein X is O and L¹ is (CR^{p}R^{q})ₙ.

Yet another aspect provides a group of compounds of formula (I), (Ia), and (Ib) wherein X is O and L¹ is (CR^{p}R^{q})ᵣ—X¹.

For each group of the compounds described above, R¹ is as described in the Summary and the Detailed Description sections.

Thus, of each groups of compounds of formula (I), (Ia), and (Ib) as described in the preceding paragraphs, examples of a subgroup include, but are not limited to, those wherein R¹ is —Si(R^{1a})₃ (e.g. —Si(C₁₋₆ alkyl)₃ wherein each C₁₋₆ alkyl is the same or different); aryl such as, but not limited to, phenyl and naphthyl (including, but not limited thereto, naphth-1-yl. naphth-2-yl), or heterocycle such as, but not limited to, dihydro-1,5-benzodioxepinyl (including, but not limited thereto, 3,4-dihydro-2H-1,5-benzodioxepin-6-yl), benzodioxolyl (including, but not limited thereto, 1,3-benzodioxol-5-yl), 2,3-dihydro-1,4-benzodioxinyl (including, but not limited thereto, 2,3-dihydro-1,4-benzodioxin-5-yl), dihydrobenzofuranyl (including, but not limited thereto, 2,3-dihydro-1-benzofuran-7-yl), and dihydroisochromenyl (including 3,4-dihydro-1H-isochromen-3-yl).

Examples of another subgroup of the compounds described in the preceding paragraphs include, but are not limited to, those wherein R¹ is phenyl.

For each of the groups and subgroups of compounds of formula (I), (Ia), and (Ib), R¹ is optionally substituted as described generally in the Summary and in the Detailed Description.

A further aspect provides a group of compounds of formula (I), (Ia), and (Ib) wherein X is S and L¹-R¹ together is formula (i).

Another aspect provides a group of compounds of formula (I), (Ia), and (Ib) wherein X is S and N(R⁴)-L¹-R¹ together is formula (ii).

Another aspect provides a group of compounds of formula (I), (Ia), and (Ib) wherein X is O and L¹-R¹ together is formula (i).

Another aspect provides a group of compounds of formula (I), (Ia), and (Ib) wherein X is O and N(R⁴)-L¹-R¹ together is formula (ii).

R³, for example, for each group and subgroup of compounds described herein, is pyridinyl, pyrimidinyl, pyrazolyl, pyridazinyl, indazolyl, 1H-pyrrolo[2,3-b]pyridinyl, 7H-pyrrolo[2,3-d]pyridinyl, and morpholinyl, each of which is optionally substituted as described in the Summary and the Detailed Description. In certain embodiments, examples of each group and subgroup of compounds of formula (I), (Ia), (Ib), (Ic), and (Ib) include those wherein R³ is optionally substituted pyridinyl (including, but not limited thereto, optionally substituted pyridin-4-yl).

Examples of present compounds include, but are not limited to, those of formula (I), (Ia), or (Ib) wherein X is S; L¹ is (CR^{p}R^{q})ₙ or (CR^{p}R^{q})ₙ—X¹. R¹ is optionally substituted phenyl, and R³ is optionally substituted pyridinyl; wherein R^{p}, R^{q}, n, m, R², R⁴, X¹, and the optional substituents of R¹ and R³ are as described in the Summary and the Detailed Description.

Another aspect of the present application relates to a group of compounds of formula (Ic) or (Id) wherein X is S, and R³ is pyridinyl, pyrimidinyl, pyrazolyl, pyridazinyl, indazolyl, 1H-pyrrolo[2,3-b]pyridinyl, 7H-pyrrolo[2,3-d]pyrimidinyl, and morpholinyl, each of which is optionally substituted as described in the Summary and the Detailed Description.

Another aspect is directed to a group of compounds of formula (Ic) or (Id) wherein X is S, and R³ is optionally substituted pyridinyl (including, but not limited thereto, optionally substituted pyridin-4-yl).

Another aspect provides a group of compounds of formula (Ic) or (Id) wherein X is O, and R³ is pyridinyl, pyrimidinyl, pyrazolyl, pyridazinyl, indazolyl, 1H-pyrrolo[2.3-b]pyridinyl, 7H-pyrrolo[2.3-d]pyrimidinyl, and morpholinyl, each of which is optionally substituted as described in the Summary and the Detailed Description.

Another aspect relates to a group of compounds of formula (Ic) or (Id) wherein X is O, and R³ is optionally substituted pyridinyl (including, but not limited thereto, optionally substituted pyridin-4-yl).

The optional substituents of R³ in each group and subgroup of compounds of formula (I), (Ia), (Ib), (Ic), and (Id) are as described in the Summary and the Detailed Description.

Examples of R², R⁴, and m for each group and subgroup of the compounds described herein are as disclosed in the Summary and the Detailed Description.

Exemplary compounds include, but are not limited to:

N-benzyl-6-pyridin-4-yl-1,3-benzothiazol-2-amine;

N-(2-phenylethyl )-6-pyridin-4-yl-1,3-benzothiazol-2-amine;

N-(1-naphthylmethyl)-6-pyridin-4-yl-1,3-benzothiazol-2-amine;

2-phenyl-2-[(6-pyridin-4-yl-1,3-benzothiazol-2-yl)amino]ethanol;

N-(3-phenylpropyl )-6-pyridin-4-yl-1,3-benzothiazol-2-amine;

N-(2-phenylethyl)-6-(1H-pyrrolo[2,3-b]pyridin-4-yl)-1,3-benzothiazol-2-amine;

N-[3,5-bis(trifluoromethyl)benzyl]-6-pyridin-4-yl-1,3-benzothiazol-2-amine;

N-(3,4-dihydro-1H-isochromen-3-ylmethyl)-6-pyridin-4-yl-1,3-benzothiazol-2-amine;

N-(1,3-benzodioxol-5-ylmethyl)-6-pyridin-4-yl-1,3-benzothiazol-2-amine;

2-methoxy-5-{[(6-pyridin-4-yl-1,3-benzothiazol-2-yl)amino]methyl}phenol;

1-phenyl-2-[(6-pyridin-4-yl-1,3-benzothiazol-2-yl)amino]ethanol;

N-(2,3-dihydro-1,4-benzodioxin-5-ylmethyl)-6-pyridin-4-yl-1,3-benzothiazol-2-amine;

2-[benzyl(6-pyridin-4-yl-1,3-benzothiazol-2-yl)amino]ethanol;

N-[(1R)-1-(3-methoxyphenyl)ethyl]-6-pyridin-4-yl-1,3-benzothiazol-2-amine;

N-benzyl-6-(3-fluoropyridin-4-yl)-1,3-benzothiazol-2-amine;

N-(3,4-dihydro-2H-1,5-benzodioxepin-6-ylmethyl)-6-pyridin-4-yl-1,3-benzothiazol-2-amine;

N-(2,3-dihydro-1,4-benzodioxin-5-ylmethyl)-6-(1H-pyrrolo[2,3-b]pyridin-4-yl)-1,3-benzothiazol-2-amine;

N-(2-ethoxybenzyl)-6-pyridin-4-yl-1,3-benzothiazol-2-amine;

N-[2-(methylthio)benzyl]-6-pyridin-4-yl-1,3-benzothiazol-2-amine;

N-[2-(difluoromethoxy)benzyl]-6-pyridin-4-yl-1,3-benzothiazol-2-amine;

N-[3-(difluoromethoxy)benzyl]-6-pyridin-4-yl-1,3-benzothiazol-2-amine;

N-[3-fluoro-5-(trifluoromethyl)benzyl]-6-pyridin-4-yl-1,3-benzothiazol-2-amine;

N-[2-(2-methylphenyl)ethyl]-6-pyridin-4-yl-1,3-benzothiazol-2-amine;

2-[methyl(6-pyridin-4-yl-1,3-benzothiazol-2-yl)amino]-1-phenylethanol;

N-[(1S)-1-phenylethyl]-6-pyridin-4-yl-1,3-benzothiazol-2-amine;

N-[(1R)-1-phenylethyl]-6-pyridin-4-yl-1,3-benzothiazol-2-amine;

N-(2-phenoxyethyl)-6-pyridin-4-yl-1,3-benzothiazol-2-amine;

N-[(1R)-1-(1-naphthyl)ethyl]-6-pyridin-4-yl-1,3-benzothiazol-2-amine;

N-[(1S)-1-(1-naphthyl)ethyl]-6-pyridin-4-yl-1,3-benzothiazol-2-amine;

N-[(1R)-1-phenylpropyl]-6-pyridin-4-yl-1,3-benzothiazol-2-amine;

N-(3-fluorobenzyl)-6-(pyridin-4-yl)benzo[d]thiazol-2-amine;

N-[(1R)-2,3-dihydro-1H-inden-1-yl]-6-pyridin-4-yl-1,3-benzothiazol-2-amine;

N-[(1S)-2,3-dihydro-1H-inden-1-yl]-6-pyridin-4-yl-1,3-benzothiazol-2-amine;

6-pyridin-4-yl-N-[3-(trimethylsilyl)propyl]-1,3-benzothiazol-2-amine;

N-benzyl-6-(1H-indazol-5-yl)-1,3-benzothiazol-2-amine;

6-(1H-indazol-5-yl)-N-(3-phenylpropyl)-1,3-benzothiazol-2-amine;

N-[(1R)-1-(2-naphthyl)ethyl]-6-pyridin-4-yl-1,3-benzothiazol-2-amine;

N-(2,3-dimethoxybenzyl)-6-pyridin-4-yl-1,3-benzothiazol-2-amine;

N-(2,5-dimethoxybenzyl)-6-pyridin-4-yl-1,3-benzothiazol-2-amine;

(2R)-2-phenyl-2-[(6-pyridin-4-yl-1,3-benzothiazol-2-yl)amino]ethanol;

N-(3-isopropoxybenzyl)-6-pyridin-4-yl-1,3-benzothiazol-2-amine;

N-[(2,2-dimethyl-2,3-dihydro-1-benzofuran-7-yl)methyl]-6-pyridin-4-yl-1,3-benzothiazol-2-amine;

2-(4-methyl-2-phenylpiperazin-1-yl)-6-pyridin-4-yl-1,3-benzothiazole;

N-(2,3-dichlorobenzyl)-6-pyridin-4-yl-1,3-benzothiazol-2-amine;

(3R)-3-phenyl-3-[(6-pyridin-4-yl-1,3-benzothiazol-2-yl)amino]propan-1-ol;

N-(2-fluorobenzyl)-6-pyridin-4-yl-1,3-benzothiazol-2-amine;

N-(2,3-difluorobenzyl)-6-pyridin-4-yl-1,3-benzothiazol-2-amine;

N-(2,3-dimethylbenzyl)-6-pyridin-4-yl-1,3-benzothiazol-2-amine;

N-[1-(2,3-dihydro-1,4-benzodioxin-5-yl)ethyl]-6-pyridin-4-yl-1,3-benzothiazol-2-amine;

N-[1-(2-chlorophenyl)ethyl]-6-pyridin-4-yl-1,3-benzothiazol-2-amine;

(2S)-2-phenyl-2-[(6-pyridin-4-yl-1,3-benzothiazol-2-yl)amino]ethanol;

3-(3-methoxyphenyl)-3-[(6-pyridin-4-yl-1,3-benzothiazol-2-yl)amino]propan-1-ol;

N-(3-methylbenzyl)-6-pyridin-4-yl-1,3-benzothiazol-2-amine;

(1S,2R)-1-[(6-pyridin-4-yl-1,3-benzothiazol-2-yl)amino]indan-2-ol;

N-[3-(hydroxyamino)-1-(3-methoxyphenyl)propyl]-6-pyridin-4-yl-1,3-benzothiazol-2-amine;

(1R,2S)-1-[(6-pyridin-4-yl-1,3-benzothiazol-2-yl)amino]indan-2-ol;

N-[(1R)-1-(2,3-dihydro-1,4-benzodioxin-5-yl)ethyl]-6-pyridin-4-yl-1,3-benzothiazol-2-amine;

N-[(1S)-1-(2,3-dihydro-1,4-benzodioxin-5-yl)ethyl]-6-pyridin-4-yl-1,3-benzothiazol-2-amine;

N-(3-chlorobenzyl)-6-pyridin-4-yl-1,3-benzothiazol-2-amine;

N-[1-(3-fluorophenyl)ethyl]-6-pyridin-4-yl-1,3-benzothiazol-2-amine;

(3R)-3-[(6-pyridin-4-yl-1,3-benzothiazol-2-yl)amino]indan-5-ol;

N-(3-fluorobenzyl)-6-(3-fluoropyridin-4-yl)-1,3-benzothiazol-2-amine;

(1R,2R)-1-[(6-pyridin-4-yl-1,3-benzothiazol-2-yl)amino]indan-2-ol;

(1S,2S)-1-[(6-pyridin-4-yl-1,3-benzothiazol-2-yl)amino]indan-2-ol;

N-(3-fluorobenzyl)-6-(2-fluoropyridin-4-yl)-1,3-benzothiazol-2-amine;

N-[(1R)-1-(3-fluorophenyl)ethyl]-6-pyridin-4-yl-1,3-benzothiazol-2-amine;

N-(3-fluorobenzyl)-6-(1H-pyrazol-5-yl)-1,3-benzothiazol-2-amine;

N-[(1R)-1-(3-ethoxyphenyl)ethyl]-6-pyridin-4-yl-1,3-benzothiazol-2-amine;

3-{[(6-pyridin-4-yl-1,3-benzothiazol-2-yl)amino]methyl}phenol;

N-(3-aminobenzyl)-6-pyridin-4-yl-1,3-benzothiazol-2-amine;

N-[4-(aminomethyl)benzyl]-6-pyridin-4-yl-1,3-benzothiazol-2-amine;

N-[3-(2-morpholin-4-ylethoxy)benzyl]-6-pyridin-4-yl-1,3-benzothiazol-2-amine;

N-(3-fluorobenzyl)-6-(2-methylpyridin-4-yl)-1,3-benzothiazol-2-amine;

3-({[6-(1H-pyrrolo[2,3-b]pyridin-4-yl)-1,3-benzothiazol-2-yl]amino}methyl)phenol;

N-{3-[2-(dimethylamino)ethoxy]benzyl}-6-pyridin-4-yl-1,3-benzothiazol-2-amine;

6-(3-fluoropyridin-4-yl)-N-[3-(2-morpholin-4-ylethoxy)benzyl]-1,3-benzothiazol-2-amine;

6-[3-(aminomethyl)pyridin-4-yl]-N-(3-fluorobenzyl)-1,3-benzothiazol-2-amine;

N-[3-(2-morpholin-4-ylethoxy)benzyl]-6-(1H-pyrrolo[2,3-b]pyridin-4-yl)-1,3-benzothiazol-2-amine;

N-{3-[3-(dimethylamino)propoxy]benzyl}-6-pyridin-4-yl-1,3-benzothiazol-2-amine;

N-(2,3-dihydro-1,4-benzodioxin-5-ylmethyl)-6-(3-fluoropyridin-4-yl)-1,3-benzothiazol-2-amine;

(2S)-2-{[6-(3-fluoropyridin-4-yl)-1,3-benzothiazol-2-yl]amino}-2-phenylethanol;

3-{[(6-pyridin-4-yl-1,3-benzothiazol-2-yl)(2-pyrrolidin-1-ylethyl)amino]methyl}phenol;

N-[3-(morpholin-4-ylmethyl)benzyl]-6-pyridin-4-yl-1,3-benzothiazol-2-amine;

N-[3-(2-piperidin-1-ylethoxy)benzyl]-6-pyridin-4-yl -1,3-benzothiazol-2-amine;

N-(3-{[(6-pyridin-4-yl-1,3-benzothiazol-2-yl)amino]methyl}phenyl)methanesulfonamide;

N-(2,3-dihydro-1,4-benzodioxin-5-ylmethyl)-6-pyrimidin-4-yl-1,3-benzothiazol-2-amine;

N-[3-(4-methylpiperazin-1-yl)benzyl]-6-pyridin-4-yl-1,3-benzothiazol-2-amine;

N-[3-(2-morpholin-4-ylethoxy)benzyl]-6-pyrimidin-4-yl-1,3-benzothiazol-2-amine;

N-[3-(2-piperidin-1-ylethoxy)benzyl]-6-pyrimidin-4-yl-1,3-benzothiazol-2-amine;

N-(3-fluorobenzyl)-6-pyrimidin-4-yl-1,3-benzothiazol-2-amine;

6-pyrimidin-4-yl-N-[3-(2-pyrrolidin-1-ylethoxy)benzyl]-1,3-benzothiazol-2-amine;

N-[3-(2-morpholin-4-ylethoxy)benzyl]-6-(7H-pyrrolo[2.3-d]pyrimidin-4-yl)-1,3-benzothiazol-2-amine;

6-(2-fluoropyridin-4-yl)-N-[3-(2-morpholin-4-ylethoxy)benzyl]-1,3-benzothiazol-2-amine;

6-(2-fluoropyridin-4-yl)-N-(2-piperidin-1-ylethyl)-1,3-benzothiazol-2-amine;

6-(2-fluoropyridin-4-yl)-N-(3-methoxybenzyl)-N-(2-piperidin-1-ylethyl)-1,3-benzothiazol-2-amine;

methyl 3-{[(6-pyridin-4-yl-1,3-benzothiazol-2-yl)amino]methyl}benzoate;

N-(3-methoxybenzyl)-N′,N′-dimethyl-N-(6-pyridin-4-yl-1,3-benzothiazol-2-yl)ethane-1,2-diamine;

N-[2-(dimethylamino)ethyl]-N-methyl-3-{[(6-pyridin-4-yl-1,3-benzothiazol-2-yl)amino]methyl}benzamide;

N-[2-(dimethylamino)ethyl]-3-{[(6-pyridin-4-yl-1,3-benzothiazol-2-yl)amino]methyl}benzamide;

N-(2-hydroxyethyl)-3-{[(6-pyridin-4-yl-1,3-benzothiazol-2-yl)amino]methyl}benzamide;

N-(2-morpholin-4-ylethyl)-3-{[(6-pyridin-4-yl-1,3-benzothiazol-2-yl)amino]methyl}benzamide;

N-(2-hydroxyethyl)-N-methyl-3-{[(6-pyridin-4-yl-1,3-benzothiazol-2-yl)amino]methyl}benzamide;

N-[(1R)-1-(3-propoxyphenyl)ethyl]-6-pyridin-4-yl-1,3-benzothiazol-2-amine;

2-(2-phenylpyrrolidin-1-yl)-6-pyridin-4-yl-1,3-benzothiazole;

6-pyridin-4-yl-2-(2-thien-2-ylpyrrolidin-1-yl)-1,3-benzothiazole;

2-[2-(4-fluorophenyl)pyrrolidin-1-yl]-6-pyridin-4-yl-1,3-benzothiazole;

2-(3-phenylpyrrolidin-1-yl)-6-pyridin-4-yl-1,3-benzothiazole;

2-[2-(5-chlorothien-2-yl)pyrrolidin-1-yl]-6-pyridin-4-yl-1,3-benzothiazole;

2-[2-(3-methoxyphenyl)pyrrolidin-1-yl]-6-pyridin-4-yl-1,3-benzothiazole;

6-pyridin-4-yl-2-[2-(1,3-thiazol-4-yl)pyrrolidin-1-yl]-1,3-benzothiazole;

2-(2-benzylpyrrolidin-1-yl)-6-pyridin-4-yl-1,3-benzothiazole;

2-[2-(5-methylthien-2-yl)pyrrolidin-1-yl]-6-pyridin-4-yl-1,3-benzothiazole;

2-[2-(3-fluorophenyl)pyrrolidin-1-yl]-6-pyridin-4-yl-1,3-benzothiazole;

2-[2-(3-chlorophenyl)pyrrolidin-1-yl]-6-pyridin-4-yl-1,3-benzothiazole;

6-pyridin-4-yl-2-[(2S)-2-thien-2-ylpyrrolidin-1-yl]-1,3-benzothiazole;

6-pyridin-4-yl-2-[(2R)-2-thien-2-ylpyrrolidin-1-yl]-1,3-benzothiazole;

3-[1-(6-pyridin-4-yl-1,3-benzothiazol-2-yl)pyrrolidin-2-yl]phenol;

3-{1-[6-(1H-pyrrolo[2,3-b]pyridin-4-yl)-1,3-benzothiazol-2-yl]pyrrolidin-2-yl}phenol;

2-{2-[3-(2-morpholin-4-ylethoxy)phenyl]pyrrolidin-1-yl}-6-pyridin-4-yl-1,3-benzothiazole;

2-{2-[3-(2-piperidin-1-ylethoxy)phenyl]pyrrolidin-1-yl}-6-pyridin-4-yl-1,3-benzothiazole;

3-[1-(6-pyrimidin-4-yl-1,3-benzothiazol-2-yl)pyrrolidin-2-yl]phenol;

3-{1-[6-(2-fluoropyridin-4-yl)-1,3-benzothiazol-2-yl]pyrrolidin-2-yl}phenol;

N-(3-fluorobenzyl)-5-pyridin-4-yl-1,3-benzothiazol-2-amine; and

N-benzyl-6-pyridin-4-yl-1,3-benzoxazol-2-amine.

It is appreciated that certain compounds described herein may exist as stereoisomers wherein at least one asymmetric or chiral center is present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30.

Individual stereoisomers (including enantiomers and diastereomers), as well as the mixtures of the enantiomers and diastereomers of said compounds (including racemates), are contemplated in the present application. Individual stereoisomers may be prepared synthetically from commercially available chiral reagents or by stereoselective or stereospecific synthetic techniques. Alternatively, the single enantiomers or diastereomers may be obtained from the preparation of racemic mixtures followed by resolution of the individual stereoisomer using methods that are known to those of ordinary skill in the art. Examples of resolution are, for example, (i) attachment of a mixture of enantiomers to a chiral auxiliary, separation of the resulting mixture of diastereomers by recrystallization or chromatography, followed by liberation of the optically pure product; or (ii) separation of the mixture of enantiomers or diastereomers on chiral chromatographic columns.

Geometric isomers may also exist in the present compounds. Various geometric isomers and mixtures thereof resulting from the disposition of substituents around a carbon-carbon double bond, a carbon-nitrogen double bond, a cycloalkyl group, or a heterocycle group are also contemplated. Substituents around a carbon-carbon double bond or a carbon-nitrogen bond are designated as being of Z or E configuration and substituents around a cycloalkyl or a heterocycle are designated as being of cis or trans configuration. The individual geometric isomers may be prepared selectively by methods known to the skilled artisan, or mixtures of the isomers may be separated by standard chromatographic or crystallization techniques.

It is to be understood that compounds disclosed hererin may exhibit the phenomenon of tautomerism. All tautomeric forms of the present compounds are contemplated.

Thus, the formulae drawings within this specification can represent only one of the possible tautomeric or stereoisomeric forms. It is to be understood that any tautomeric or stereoisomeric form, and mixtures thereof are encompassed, and is not to be limited merely to any one tautomeric or stereoisomeric form utilized within the naming of the compounds or formulae drawings.

### c. BIOLOGICAL DATA

### (i) In Vitro Methods

ROCK-2 Inhibitory Assay

Certain compounds were tested for their ability to inhibit N-terminal His6-tagged recombinant human ROCK-2 residues 11-552 expressed by baculovirus in Sf21 cells (Upstate). In 384-well v-bottom polypropylene plates (Axygen). 1 nM (final concentration) in 10 μL recombinant N-terminal His6-tagged recombinant human ROCK-2 residues 11-552 expressed by baculovirus in Sf21 cells (Upstate) was mixed with 2 μM (final concentration) in 10 μL biotinylated peptide substrate (biotin-Aha-K-E-A-K-E-K—R-Q-E-Q-I-A-K—R—R—R-L-S—S-L-R-A-S-T-S—K—S-G-G-S-Q-K) (Genemed), and various concentration of inhibitor (2% DMSO final) in reaction buffer (25 mM HEPES. pH 7.5, 0.5 mM DTT, 10 mM MgCl₂, 100 μM Na₃VO₄, 0.075 mg/mL Triton X-100), and the reaction was initiated by addition of 5 μM unlabelled ATP containing 0.01 μCi[³³P]-ATP (Perkin Elmer). The reaction was quenched after 1 hour by the addition of 50 μL stop buffer (50 mM EDTA, 2M NaCl final concentration). 80 μL of the stopped reactions were transferred to 384-well streptavidin-coated FlashPlates (Perkin Elmer), incubated 10 minutes at room temperature, washed 3 times with 0.05% Tween-20/PBS using an ELX-405 automated plate washer (BioTek), and counted on a TopCount Scintillation Plate Reader (Packard).

ROCK-1 Inhibitory Assay

Certain compounds were tested for their ability to inhibit N-terminal His6-tagged, recombinant human ROCK-1 amino acids 17-535 expressed by baculovirus in Sf21 cells (Upstate). In 384-well v-bottom polypropylene plates (Axygen), 2 nM (final concentration) in 10 μL recombinant N-terminal His6-tagged, recombinant human ROCK-1 amino acids 17-535 expressed by baculovirus in Sf21 cells (Upstate) in reaction buffer was mixed with 2 μM (final concentration) biotinylated peptide substrate (biotin-Aha-V—R—R-L-R—R-L-T-A-R-E-A-A) (Genemed), and various concentration of inhibitor (2% DMSO final) in 10 μL reaction buffer (25 mM HEPES, pH 7.5, 0.5 mM DTT, 10 mM MgCl₂, 100 AM Na₃VO₄, 0.075 mg/ml Triton X-100), and the reaction was initiated by addition of 5 μM unlabelled ATP containing 0.01 μCi[³³P]-ATP (Perkin Elmer). The reaction was quenched after 1 hour by the addition of 50 μL stop buffer (50 mM EDTA, 2M NaCl final concentration). 80 μL of the stopped reactions were transferred to 384-well streptavidin-coated FlashPlates (Perkin Elmer), incubated 10 minutes at room temperature, washed 3 times with 0.05% Tween-20/PBS using an ELX-405 automated plate washer (BioTek), and counted on a TopCount Scintillation Plate Reader (Packard).

### ii) In Vivo Data

Determination of Antinociceptive Effect: Models for Neuropathic Pain

Spinal Nerve (L5/L6) Ligation Model of Neuropathic Pain: As described by Kim and Chung (Kim S. H.; Chung J. M.; An experimental model for peripheral neuropathy produced by segmental spinal nerve ligation in the rat. Pain 1992, 50, 355-363), a 1.5 cm incision was made dorsal to the lumbosacral plexus. In anesthetized rats, the paraspinal muscles (left side) were separated from the spinous processes, the L5 and L6 spinal nerves isolated, and tightly ligated with 3-0 silk threads. Following hemostasis, the wound was sutured and coated with antibiotic ointment. The rats were allowed to recover and then placed in a cage with soft bedding for 14 days before behavioral testing for mechanical allodynia.

Sciatic Nerve Ligation Model of Neurolpathic Pain: As described by Bennett and Xie (Bennett G. J.; Xie Y-K.; A peripheral mononeuropathy in rat that produces disorders of pain sensation like those seen in man. Pain 1988, 33, 87-107), in anesthetized rats, a 1.5 cm incision was made 0.5 cm below the pelvis and the biceps femoris and the gluteous superficialis (right side) were separated. The sciatic nerve was exposed, isolated, and four loose ligatures (5-0 chromic catgut) with 1 mm spacing were placed around it. The rats were allowed to recover and then placed in a cage with soft bedding for 14 days before behavioral testing for mechanical allodynia as described above. In addition, animals were also tested for cold allodynia by dipping their hind paw in a cold-water bath (4.5° C.) and determining the paw withdrawal latency.

Selected compounds, dosed either i.p. or p.o. demonstrated >30% inhibition of tactile allodynia in the Chung and Bennett models (Chaplan S R, Bach F W, Pogrel J W, Chung J M & Yaksh T L (1994), Journal of Neuroscience Methods, 53(1):55-63.) of neuropathic pain at doses ranging from 1-150 mg/kg.

Certain compounds tested were found to inhibit human ROCK-2 and human ROCK-1 kinases, exhibiting an IC₅₀ of about 1.0 μM to about 1 nM.

### d. METHODS OF USING THE COMPOUNDS

Compounds described herein have ROCK antagonistic activity. Because of their profile, the compounds can be used for treating diseases which respond to the influencing of ROCK activity, i.e. they are effective for treating those medical disorders or diseases in which exerting an influence on (modulating) the ROCK activity leads to an improvement in the clinical picture or to the disease being cured. Examples of these diseases are given above.

The disorders which can be treated in accordance with the invention include the diseases listed in the introductory part, e.g. cardiovascular diseases such as hypertension, chronic and congestive heart failure, cardiac hypertrophy, chronic renal failure, cerebral vasospasm after subarachnoid bleeding, pulmonary hypertension and ocular hypertension, cancer and tumor metastasis, asthma, male erectile dysfunctions, female sexual dysfunctions, over-active bladder syndrome, preterm labor, ischemia reperfusion, myocardial infarction, restenosis, atherosclerosis, graft failure, CNS disorders, such acute neuronal injury, e.g. spinal chord injury, traumatic brain injury and stroke, Parkinson's disease and Alzheimer's disease, inflammatory and demyelating diseases such as multiple sclerosis, acute and chronic pain, rheumatoid arthritis, osteoarthritis, osteoporosis, irritable bowel syndrome and inflammatory bowel disease, amyotrophic lateral sclerosis, HIV-1 encephalitis, virus and bacterial infections, insulin resistance, diabetes, cognitive dysfunctions, such as the above-mentioned Alzheimer's disease, vascular dementia and other dementia forms, glaucoma, psoriasis, retinopathy, and benign prostatic hypertrophy. In particular the disorders are cancer, pain, asthma, cognitive dysfunctions, in particular vascular dementia and Alzheimer's disease, multiple sclerosis, rheumatoid arthritis and spinal cord injuries.

Within the meaning of the invention, a treatment also includes a preventive treatment (prophylaxis), in particular as relapse prophylaxis or phase prophylaxis, as well as the treatment of acute or chronic signs, symptoms and/or malfunctions. The treatment can be orientated symptomatically, for example as the suppression of symptoms. It can be effected over a short period, be orientated over the medium term or can be a long-term treatment, for example within the context of a maintenance therapy.

The treatment is effected by means of single or repeated daily administration, where appropriate together, or alternating, with other active compounds or active compound-containing preparations.

Within the context of the treatment, the use according to the invention of the described compounds involves a method. In this method, an effective quantity of one or more compounds, as a rule formulated in accordance with pharmaceutical and veterinary practice, is administered to the individual to be treated, preferably a mammal, in particular a human being, productive animal or domestic animal. Whether such a treatment is indicated, and in which form it is to take place, depends on the individual case and is subject to medical assessment (diagnosis) which takes into consideration signs, symptoms and/or malfunctions which are present, the risks of developing particular signs, symptoms and/or malfunctions, and other factors.

Present compounds can also be administered as a pharmaceutical composition comprising therapeutically effective amounts of the compounds of interest in combination with one or more pharmaceutically acceptable carriers. The phrase “therapeutically effective amount” of the present compounds means sufficient amount of the compounds to achieve the desired therapeutic response for a particular patient, compositions and mode of administration, at a reasonable benefit/risk ratio applicable to any medical treatment. It will be understood, however, that the total daily usage of the compounds and compositions will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved.

The total daily dose of the compounds administered to a human or lower animal may range from about 0.003 to about 30 mg/kg/day. For purposes of oral administration, more preferable doses can be in the range of from about 0.01 to about 10 mg/kg/day. If desired, the effective daily dose can be divided into multiple doses for purposes of administration; consequently, single dose compositions may contain such amounts or submultiples thereof to make up the daily dose.

### e. PHARMACEUTICAL COMPOSITIONS

Further provided are pharmaceutical compositions capable of treating protein kinases associated conditions, in particular, Rho kinase (ROCK) mediated conditions, as described above. Pharmaceutical compositions comprising compounds of interest, or solvates or salts thereof may be formulated by employing conventional solid or liquid vehicles or diluents, as well as pharmaceutically acceptable additives of a type appropriate to the mode of administration (e.g. excipients, binders, preservatives, stabilizers, flavors, etc) according to techniques such as those well known in the art of pharmaceutical formulations.

The compounds described herein may be administered by any means suitable for the condition to be treated, which may depend on the need of site-specific treatment or quantity of drug to be delivered.

The pharmaceutical compositions may be administered to humans and other mammals orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil, glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Exemplary compositions for rectal or vaginal administration include suppositories which can be prepared by mixing the compounds of interest with suitable non-irritating carriers or carriers such as cocoa butter, polyethylene glycol or a suppository wax which are solid at room temperature but liquid at body temperature and therefore melt in the rectum or vaginal cavity and release the active compound.

The compounds described herein can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to the compounds of formula (I), (Ia), or (Ib), stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

Dosage forms for topical administration of the compounds include powders, sprays, ointments and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

The compounds described herein can be used in the form of pharmaceutically acceptable salts derived from inorganic or organic acids. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio.

Pharmaceutically acceptable salts are well known in the art. For example, S. M. Berge et al. describe pharmaceutically acceptable salts in detail in (J. Pharmaceutical Sciences, 1977, 66: 1 et seq). The salts can be prepared in situ during the final isolation and purification of the compounds or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propioonate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts can be prepared in situ during the final isolation and purification of compounds by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

The term “pharmaceutically acceptable prodrug” or “prodrug” as used herein, represents those prodrugs of the compounds which are, within the scope of sound medical judgment, suitable for use in contact with the tissues of humans and lower animals without undue toxicity, irritation, allergic response, and the like, commensurate with a reasonable benefit/risk ratio, and effective for their intended use.

The present application contemplates compounds formed by synthetic means or formed by in vivo biotransformation of a prodrug.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### f. GENERAL SYNTHESIS

This invention is intended to encompass compounds described herein when prepared by synthetic processes or by metabolic processes. Preparation of the compounds by metabolic processes includes those occurring in the human or animal body (in vivo) or processes occurring in vitro.

The compounds described herein may be prepared by a variety of processes well known for the preparation of compounds of this class. For example, compounds of formula (I) wherein the groups X, m, R¹, R², R³, R⁴, and L¹ have the meanings as set forth in the summary section unless otherwise noted, can be generally prepared according to Schemes 1-4 and knowledge of one skilled in the art.

As used in the descriptions of the schemes and the examples, certain abbreviations are intended to have the following meanings: HPLC for high performance liquid chromatography or high pressure liquid chromatography, (dppf) for [1,1′-bis(diphenylphosphino)ferrocene; DMSO for dimethylsulfoxide, triflate for trifluoroacetate; OMs or mesylate for methanesulfonate, OTs or tosylate for p-toluenesulfonate, and TFA for trifluoroacetic acid.

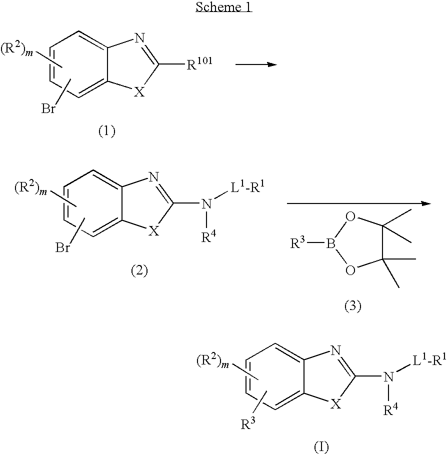

The preparation of the compounds of general formula (I) is shown in Scheme 1. Reaction of an appropriately substituted bicyclic rings of formula (1) wherein R¹⁰¹ is leaving group such as, but not limited to, chloro, triflate, or tosylate, with amines of formula N(R⁴)(H)L¹R¹ in an inert solvent such as, but not limited to, dimethylsulfoxide, toluene, and the like, in the presence of a base, affords intermediates of formula (2). An example of a suitable base for the reaction includes, but is not limited to, diethylisopropyl amine. The reaction may be conducted at an elevated temperature, for example, at about 80° C. to about 130° C. Treatment of (2) with an appropriate boronic ester such as those of formula (3), or trialkyl tin reagents of formula R³Sn(R¹⁰²)₃ wherein R¹⁰² is alkyl and each R¹⁰² can be the same or different, under coupling reaction conditions known to a skilled artisan, provides compounds of formula (Ia). For example, the coupling between (2) and (3) can be effected in the presence of a palladium agent such as, but not limited to, tetrakis(triphenylphosphine)palladium(0) or PdCl₂(dppf)₂.CH₂Cl₂, and a base such as, but not limited to, potassium or cesium carbonate, and in a solvent such as, but not limited to, dioxane, water, ethanol, methanol, dimethoxyethane, or mixtures thereof, and at elevated temperature (for example, between about 80 to about 120° C.).

The amines of formula N(R⁴)(H)L¹R¹ may be prepared by synthetic methods known in the art. For example, by reductive amination of an appropriately substituted amide, or by reduction of an appropriately substituted nitrile or amide, or by (a) displacement of an appropriately substituted halide with sodium azide, followed by (b) reduction of the resulting azide.

Alternatively, compounds of formula (I) may be prepared using general procedures as outlined in Scheme 2.

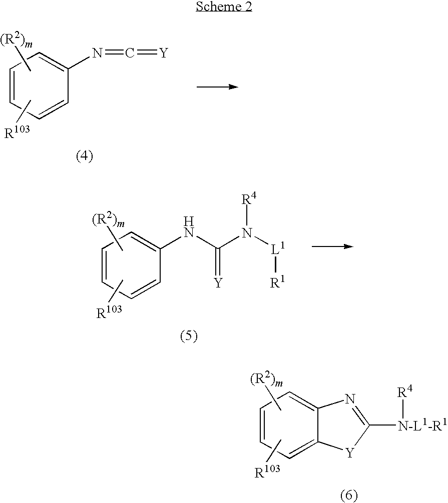

An appropriately substituted isothiocyanate or isocyanate wherein Y is S or O, and R¹⁰³ is halogen, tosylate, triflate, or R³, can be treated with amines of formula N(R⁴)(H)L¹R¹ in an inert solvent such as ether, to provide the thiourea of urea of formula (5) wherein Y and R¹⁰³ are as defined herein. The resulting thiourea or urea may be isolated or can be cyclized without isolation. Cyclization of intermediates (5) can be conducted in the presence of bromine in a solvent such as tolune, and at a temperature ranging from about room temperature to about 70° C., to provide compounds of formula (6). The cyclization can also be effected by the presence of other oxidizing agent such as, but not limited to, sodium hypochlorate, in an inert solvent such as dichloromethane.

The isothiocyanate or isocyanate are commercially available or prepared from the corresponding aniline by treatment with thiophosgene or phosgene, in the presence of a base, such as, sodium carbonate, in an inert solvent.

Compounds of formula (6) wherein R¹⁰³ is other than R³ can be transformed to compounds of formula (6) wherein R¹⁰³ is other than R³ using the synthetic methods for the conversion of (2) to (1) in Scheme 1.

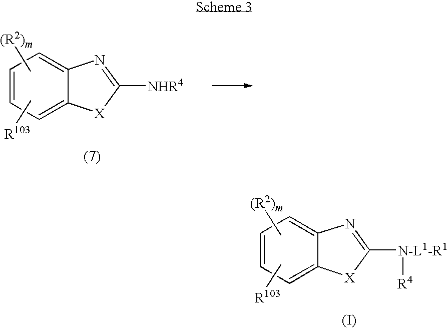

Compounds of general formula (I) can also be prepared via reductive amination reaction as shown in Scheme 4. Standard reductive amination conditions can be employed for the treatment of (7) with appropriate aldehydes of formula R¹L¹C(O)H, to provide compounds of formula (1).

Compounds described herein may also be useful intermediates for the preparation of additional compounds of the present application. For example, primary and secondary amines may be acylated, alkylated, or coupled carboxylic acids or amino acids under standard peptide coupling conditions. Furthermore, ester moieties may be reduced to the corresponding alcohols or converted to amides under standard conditions. Alcohols may be activated and displaced by a number of nucleophiles to provide other compounds of formula (I).

It will be appreciated that the synthetic schemes and specific examples as illustrated in the Examples section are illustrative and are not to be read as limiting the scope of the invention as it is defined in the appended claims. All alternatives, modifications, and equivalents of the synthetic methods and specific examples are included within the scope of the claims.

Optimum reaction conditions and reaction times for each individual step may vary depending on the particular reactants employed and substituents present in the reactants used. Unless otherwise specified, solvents, temperatures and other reaction conditions may be readily selected by one of ordinary skill in the art. Specific procedures are provided in the Examples section. Reactions may be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or may be prepared by one skilled in the art from commercially available materials using methods described in the chemical literature.

The skilled artisan will also appreciate that not all of the substituents in the compounds of formula (I) will tolerate certain reaction conditions employed to synthesize the compounds. Routine experimentations, including appropriate manipulation of the reaction conditions, reagents and sequence of the synthetic route, protection of any chemical functionality that may not be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety.

Furthermore, the skilled artisan will appreciate that in many circumstances, the order in which moieties are introduced may not be critical. The particular order of steps required to produce the compounds of formula (I) is dependent upon the particular compounds being synthesized, the starting compound, and the relative lability of the substituted moieties. Thus, synthesis of the present compounds may be accomplished by methods analogous to those described in the synthetic schemes described hereinabove and in specific examples, and routine experimentations, including appropriate manipulation of the reaction conditions, reagents and sequence of the synthetic route are within the scope of the invention.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it may be obtained by carrying out one of the above procedures using a pure geometric isomer as a starting material, or by resolution of a mixture of the geometric isomers of the compound or intermediates using a standard procedure such as chromatographic separation.

The following Examples may be used for illustrative purposes and should not be deemed to narrow the scope of the invention.

### g. EXAMPLES

All final products were purified by preparative HPLC on a Phenomenex Luna C8(2) 5 urn 100 Å AXIA column (30 mm×75 mm). A gradient of acetonitrile (A) and 0.1% 30 trifluoroacetic acid in water (B) was used, at a flow rate of 70 mL/min (0-0.5 min 10% A, 0.5-12.0 min linear gradient 10-95% A, 12.0-15.0 min 95% A, 15.0-17.0 min linear gradient 95-10% A). Samples were injected in 2.5 mL dimethyl sullfoxide:methanol (1:1). A custom purification system was used, consisting of the following modules: Waters LC4000 preparative pump; Waters 996 diode-array detector: Waters 717+ autosampler; Waters SAT/IN module, Alltech Varex III evaporative light-scattering detector; Gilson 506C interface box; and two Gilson FC204 fraction collectors. The system was controlled using Waters Millennium32 software, automated using an Abbott developed Visual Basic application for fraction collector control and fraction tracking. Fractions were collected based upon UV signal threshold and selected fractions subsequently analyzed by flow injection analysis mass spectrometry using positive APCI ionization on a Finnigan LCQ using 70:30 methanol:10 mM NH₄OH (aqueous) at a flow rate of 0.8 mL/min. Loop-injection mass spectra were acquired using a Finnigan LCQ running LCQ Navigator 1.2 software and a Gilson 215 liquid handler for fraction injection controlled by an Abbott developed Visual Basic application.

### Example 1

### N-benzyl-6-pyridin-4-yl-1,3-benzothiazol-2-amine

### Example 1A

6-bromo-2-chlorobenzo[d]thiazole (0.5 g, 2.012 mmol) was added to a reaction flask containing diisopropylethylamine (0.7 mL, 2.213 mmol) and benzyl amine (0.237 g, 2.213 mmol) in dimethylsulfoxide (5 mL). The reaction was heated at 100° C. under a steady stream of N₂ for 12 hours. The cooled reaction was then poured into water (20 mL) and the precipitated tan solid was filtered and washed successively with water (3×20 mL) and hexanes (3×20 mL) yielding 0.663 g of Example 1A.

### Example 1B

### N-benzyl-6-pyridin-4-yl-1,3-benzothiazol-2-amine

An aqueous solution of 2 M cesium carbonate (1.2 mL, 2.8 mmol) was added to a reaction flask containing the product from Example 1A (0.2 g, 0.69 mmol), 4-(4,4,5,5-tetramethyl-1,3,2-dioxaborolan-2-yl)pyridine (0.156 g, 0.76 mmol) and PdCl₂(dppf)₂.CH₂Cl₂ (0.48 g, 0.07 mmol). The reaction mixture was heated at 95° C. under N₂ for 12 hours. The cooled reaction was diluted with CH₂Cl₂ (10 mL) and washed with saturated NaHCO₃ (2×20 mL) and water (2×10 mL). The organic layer was concentrated and the residue was purified via HPLC to provide 0.053 g of the title compound as a TFA salt. ¹H NMR (300 MHz, CDCl₃) δ ppm 4.66 (s, 2H) 7.33-7.48 (m, 5H) 7.62-7.76 (m, 3H) 7.89 (s, 2H) 8.74 (d, J=6.35 Hz, 2H).

### Example 2

### N-(2-phenylethyl)-6-pyridin-4-yl-1,3-benzothiazol-2-amine

### Example 2A

Example 2A (0.495 g) was synthesized following the procedure for Example 1A, substituting 2-phenylethanamine for benzyl amine. Example 2A was used in the next step without further purification.

### Example 2B

### N-(2-phenylethyl)-6-pyridin-4-yl-1,3-benzothiazol-2-amine

The TFA salt of the title compound (0.024 g) was synthesized following the procedure from Example 1B, substituting Example 2A for Example 1A. ¹H NMR (300 MHz, DMSO-d₆) δ ppm 2.91 (t, J=7.29 Hz, 2H) 3.49-3.68 (m, 2H) 7.11-7.41 (m, 5H) 7.84 (d, J=1.70 Hz, 2H) 8.12 (d, J=6.10 Hz, 2H) 8.79 (d, J=6.44 Hz, 2H) 9.05 (s, 1H)

### Example 3

### N-(1-naphthylmethyl)-6-pyridin-4-yl-1,3-benzothiazol-2-amine

### Example 3A

Example 3A (0.495 g) was synthesized following the procedure of Example 1A, substituting naphthalene-1-ylmethanamine for benzyl amine. Example 3A was used in the next step without farther purification.

### Example 3B

### N-(1-naphthylmethyl)-6-pyridin-4-yl-1,3-benzothiazol-2-amine

The TFA salt of the title compound (0.030 g) was synthesized procedure from Example 1B, substituting Example 3A for Example 1A. ¹H NMR (500 MHz, DMSO-d₆) δ ppm 5.88 (s, 2H) 7.47-7.67 (m, 5H) 7.83-7.94 (m, 2H) 7.99 (d, J=7.32 Hz, 1H) 8.20-8.31 (m 3H) 8.41 (d, J=2.14 Hz, 1H) 8.77 (d, J=6.71 Hz, 2H).

### Example 4

### 2-phenyl-2-[(6-pyridin-4-yl-1,3-benzothiazol-2-yl)amino]ethanol

### Example 4A

Example 4A (0.220 g) was synthesized following the procedure for Example 1A, substituting 2-amino-2-phenylethanol for benzyl amine. Example 4A was used in the next step without further purification.

### Example 4B

### 2-phenyl-2-[(6-pyridin-4-yl-1,3-benzothiazol-2-yl)amino]ethanol

The TFA salt of the title compound (0.032 g) was synthesized following the procedure for Example 1B, substituting Example 4A for Example 1A. ¹H NMR (500 MHz, CD₃OD) δ ppm 3.77-3.93 (m, 2H) 5.03-5.14 (m, 1H) 7.26-7.51 (m, 5H) 7.57 (d, J=8.54 Hz, 1H) 7.92 (dd, J=8.54, 1.83 Hz, 1H) 8.26-8.40 (m, 3H) 8.73 (d, J=7.02 Hz, 2H)

### Example 5

### N-(3-phenylpropyl)-6-pyridin-4-yl-1,3-benzothiazol-2-amine

### Example 5A

Example 5A (0.35 g) was synthesized following the procedure for Example 1A, substituting 3-phenylpropan-1-amine for benzyl amine. Example 5A was used in the next step without further purification.

### Example 5B

### N-(3-phenylpropyl)-6-pyridin-4-yl-1,3-benzothiazol-2-amine

The TFA salt of the title compound (0.035 g) was synthesized following the procedure for Example 1B, substituting Example 5A for Example 1A. ¹H NMR (300 MHz, DMSO-d₆) δ ppm 1.73-2.01 (m, 2H) 2.60-2.82 (m, 2H) 3.42 (dd, J=11.70, 6.61 Hz, 2H) 6.91-7.40 (m, 5H) 7.53 (d, J=8.82 Hz, 1H) 7.89 (dd, J=8.48, 2.03 Hz, 1H) 8.19 (d, J=6.78 Hz, 2H) 8.42 (d, J=2.03 Hz, 1H) 8.50 (s, 1H) 8.79 (d, J=6.10 Hz, 2H).

### Example 6

### N-(2-phenylethyl)-6-(1H-pyrrolo[2,3-b]pyridin-4-yl)-1,3-benzothiazol-2-amine

### Example 6A

### (1-(triisopropylsilyl)-1H-pyrrolo[2,3-b]pyridin-4-ylboronic acid)

To a 0° C. solution of 4-bromo-1H-pyrrolo[2,3-b]pyridine (6.99 g, 35.5 mmol) in tetrahydrofuran (71.0 mL) was added sodium hydride (3.12 g, 78 mmol) portionwise. The reaction mixture was stirred for 15 minutes at 0° C. under a N₂ atmosphere, after which triisopropylchlorosilane (15.03 mL, 71.0 mmol) was added. The mixture was heated at reflux for 12 hours. After cooling to room temperature, the reaction mixture was quenched by the addition of saturated aqueous NH₄Cl, and then extracted twice with hexanes. The combined organic layers were dried over Na₂SO₄, filtered, and concentrated in vacuo. The recovered 4-bromo-1-(triisopropylsilyl)-1H-pyrrolo[2,3-b]pyridine (12.54 g, 35.5 mmol) was directly dissolved in tetrahydrofuran (178 mL) and n-butyllithium (66.6 mL, 107 mmol) was added dropwise at −78° C. over 5 minutes, followed by the addition of trimethyl borate (8.34 mL, 71.0 mmol). The mixture was stirred for 45 min at −78° C., quenched by saturated aqueous NH₄Cl, diluted with water, and extracted 3× with hexanes. The combined organic layers were dried over Na₂SO₄, filtered, and concentrated in vacuo yielding 4.1 g of the title compound, which was used directly in the next step.

### Example 6B

### N-(2-phenylethyl)-6-(1H-pyrrolo[2,3-b]pyridin-4-yl)-1,3-benzothiazol-2-amine

An aqueous solution of 2 M cesium carbonate (1.3 mL, 2.5 mmol) was added to a reaction flask containing the product from Example 2A (0.2 g, 0.69 mmol), Example 6A (0.2 g, 0.638 mmol) and Pd(dppf)Cl₂.CH₂Cl₂ (0.051 g, 0.063 mmol). The reaction mixture was heated at 95° C. under N₂ for 12 hours. The cooled reaction mixture was diluted with CH₂Cl₂ (10 mL) and washed sequentially with 2N HCl (3×10 mL), saturated NaHCO₃ (5×20 mL) and water (2×10 mL). The concentrated organic layer was purified via HPLC, yielding 0.045 g of the title compound. ¹H NMR (300 MHz, CDCl₃) δ ppm 4.66 (s, 2H) 7.33-7.48 (m, 5H) 7.62-7.76 (m, 3H) 7.89 (s, 2H) 8.74 (d, J=6.35 Hz, 2H).

### Example 7

### N-[3,5-bis(trifluoromethyl)benzyl]-6-pyridin-4-yl-1,3-benzothiazol -2-amine

### Example 7A

Example 7A (0.67 g) was prepared following the procedure for Example 1A, substituting 3,5-bis(trifluormethyl)benzylamine for benzyl amine. Example 7A was used in the next step without further purification.

### Example 7B

### N-[3,5-bis(trifluoromethyl)benzyl]-6-pyridin-4-yl -1,3-benzothiazol-2-amine

The TFA salt of the title compound (0.035 g) was synthesized following procedure for Example 1B, substituting Example 7A for Example 1A. ¹H NMR (300 MHz, DMSO-d₆) δ ppm 4.86 (d, J=5.55 Hz, 2H) 4.86 (d, J=5.55 Hz, 1H) 7.56 (d, J=8.33 Hz, 1H) 7.82-8.23 (m, 5H) 8.45 (d, J=1.98 Hz, 1H) 8.80 (d, J=6.74 Hz, 2H) 9.01 (s, 1H).

### Example 8

### N-(3,4-dihydro-1H-isochromen-3-ylmethyl)-6-pyridin-4-yl-1,3-benzothiazol-2-amine

### Example 8A

Example 8A (0.38 g) was prepared following the procedure for Example 1A, substituting isochroman-3-ylmethanamine for benzyl amine. Example 8A was used in the next step without further purification.

### Example 8B

### N-(3,4-dihydro-1H-isochromen-3-ylmethyl)-6-pyridin-4-yl-1,3-benzothiazol-2-amine

The TFA salt of the title compound (0.155 g) was synthesized using procedure from Example 1B, substituting Example 8A for Example 1A. 1H NMR (300 MHz, DMSO-d₆) δ ppm 2.63-2.83 (m, 1H) 2.89 (s, 1H) 3.58-3.84 (m, 2H) 3.92-4.20 (m, 1H) 4.99 (dd, J=8.13, 2.58Hz, 2H) 7.08-7.38 (m, 4H) 7.57 (d, J=8.33 Hz, 1H) 7.94 (dd, J=8.72, 1.98 Hz, 1H) 8.28 (d, J=6.74 Hz, 2H) 8.46 (d, J=1.98 Hz, 1H) 8.68 (s, 1H) 8.83 (d, J=6.74 Hz, 2H).

### Example 9

### N-(1,3-benzodioxol-5-ylmethyl)-6-pyridin-4-yl-1,3-benzothiazol-2-amine

### Example 9A

Example 9A (0.59 g) was prepared following the procedure for Example 1A, substituting benzo[d][1,3]dioxol-5-ylmethanamine for benzyl amine. Example 9A was used in the next step without further purification.

### Example 9B

### N-(1,3-benzodioxol-5-ylmethyl)-6-pyridin-4-yl-1,3-benzothiazol-2-amine

The TFA salt of the title compound (0.012 g) was synthesized following the procedure from Example 1B, substituting Example 9A for Example 1A. ¹H NMR (500 MHz, CD₃OD) δ ppm 4.59 (s, 2H) 5.93 (s, 2H) 6.80 (d, J=7.63 Hz, 1H) 6.85-6.97 (m, 2H) 7.62 (d, J=8.54 Hz, 1H) 7.96 (dd, J=8.54, 2.14 Hz, 1H) 8.24-8.45 (m, 3H) 8.75 (d, J=7.02 Hz, 2H).

### Example 10

### 2-methoxy-5-{[(6-pyridin-4-yl-1,3-benzothiazol-2-yl)amino]methyl}phenol

### Example 10A

Example 10A (0.21 g) was prepared following the procedure for Example 1A, substituting 5-(aminomethyl)-2-methoxyphenol for benzyl amine. Example 10A was used in the next step without further purification.

### Example 10B

### 2-methoxy-5-{[(6-pyridin-4-yl-1,3-benzothiazol-2-yl)amino]methyl}phenol

Example 10B (0.009 g) was synthesized using the procedure from Example 1B, substituting Example 10A for Example 1A. ¹H NMR (500 MHz, CD₃OD) δ ppm 2.65 (s, 1H) 3.83 (s, 3H) 4.56 (s, 2H) 6.78-6.96 (m, 3H) 7.61 (d, J=8.54 Hz, 1H) 7.95 (dd, J=8.70, 1.98 Hz, 1H) 8.27-8.42 (m, 3H) 8.74 (d, J=5.80 Hz, 2H).

### Example 11

### 1-phenyl-2-[(6-pyridin-4-yl-1,3-benzothiazol-2-yl)amino]ethanol

### Example 11A

Example 11A (0.34 g) was synthesized following the procedure for Example 1A, substituting 2-amino-1-phenylethanol for benzyl amine. Example 11A was used in the next step without further purification.

### Example 11B

### 1-phenyl-2-[(6-pyridin-4-yl-1,3-benzothiazol-2-yl)amino]ethanol

The TFA salt of the title compound (0.160 g) was synthesized using the procedure from Example 1B, substituting Example 11A for Example 1A. ¹H NMR (400 MHz, DMSO-d₆) δ ppm 2.08 (d, J=1.23 Hz, 1H) 3.53 (dd, J=13.04, 8.13 Hz, 1H) 3.65 (d, J=3.07 Hz, 1H) 4.87 (dd, J=7.67, 4.60 Hz, 1H) 6.98-7.48 (m, 2H) 7.56 (d, J=7.98 Hz, 1H) 7.95 (d, J=8.59 Hz, 1H) 8.33 (d, J=6.14 Hz, 1H) 8.47 (s, 1H) 8.71 (s, 1H) 8.86 (d, J=6.14 Hz, 2H).

### Example 12

### N-(2,3-dihydro-1,4-benzodioxin-5-ylmethyl)-6-pyridin-4-yl-1,3-benzothiazol-2-amine

### Example 12A

Example 12A (0.66 g) was prepared following the procedure for Example 1A, substituting (2,3-dihydrobenzo[b][1,4]dioxin-5-yl)methanamine for benzyl amine. Example 1A was used in the next step without further purification.

### Example 12B

### N-(2,3-dihydro-1,4-benzodioxin-5-ylmethyl)-6-pyridin-4-yl-1,3-benzothiazol-2-amine

The TFA salt of the title compound (0.118 g) was synthesized using the procedure from Example 1B, substituting Example 12A for Example 1A. ¹H NMR (300 MHz, CD₃OD) δ ppm 4.18-4.40 (m, 4H) 4.66 (s, 1H) 6.55-6.85 (m, 2H) 6.85-7.03 (m, 1H) 7.62 (d, J=8.72 Hz, 1H) 7.96 (dd, J=8.72, 1.98 Hz, 1H) 8.26-8.42 (m, 3H) 8.75 (d, J=6.74Hz, 2H).

### Example 13

### 2-(benzyl(6-(pyridin-4-yl)benzo[d]thiazol-2-yl)amino)ethanol

### Example 13A

Example 13A (0.33 g) was synthesized following the procedure for Example 1A, substituting 2-(benzylamino)ethanol for benzyl amine. Example 13A was used in the next step without further purification.

### Example 13B

### 2-(benzyl(6-(pyridin-4-yl)benzo[d]thiazol-2-yl)amino)ethanol

The TFA salt of the title compound (0.002 g) was synthesized following the procedure from Example 1B, substituting Example 13A for Example 1A.

### Example 14

### N-[(1R)-1-(3-methoxyphenyl)ethyl]-6-pyridin-4-yl-1,3-benzothiazol-2-amine

### Example 14A

Example 14A (0.45 g) was prepared following the procedure for Example 1A, substituting (R)-1-(3-methoxyphenyl)ethanamine for benzyl amine. Example 14A was used in the next step without further purification.

### Example 14B

### N-[(1R)-1-(3-methoxyphenyl)ethyl]-6-pyridin-4-yl-1,3-benzothiazol-2-amine

The TFA salt of the title compound (0.047 g) was synthesized using the procedure from Example 1B, substituting Example 14A for Example 1A. ¹H NMR (300 MHz, CD₃OD) δ ppm 1.61 (d, J=7.12 Hz, 3H) 3.79 (s, 3H) 5.06 (d, J=6.78Hz, 1H) 6.75-6.90 (m, 1H) 6.92-7.06 (m, 2H) 7.27 (t, J=8.14 Hz, 1H) 7.58 (d, J=8.48 Hz, 1H) 7.92 (dd, J=8.82, 2.03 Hz, 1H) 8.24-8.40 (m, 3H) 8.74 (d, J=7.12 Hz, 2H).

### Example 15

### N-benzyl-6-(3-fluoropyridin-4-yl)-1,3-benzothiazol-2-amine

The TFA salt of the title compound (0.047 g) was synthesized following the procedure from Example 1B, substituting 3-fluoropyridin-4-ylboronic acid for 4-(4,4,5,5-tetramethyl-1,3,2-dioxaborolan-2-yl)pyridine. ¹H NMR (300 MHz, CD₃OD) δ ppm 4.70 (s, 2H) 7.16-7.51 (m, 4H) 7.22-7.49 (m, 1H) 7.51-7.65 (m, 1H) 7.64-7.84 (m, 2H) 8.05 (s, 1H) 8.46 (d, J=5.42 Hz, 1H) 8.58 (d, J=3.05 Hz, 1H).

### Example 16

### N-(3,4-dihydro-2H-1,5-benzodioxepin-6-ylmethyl)-6-pyridin-4-yl-1,3-benzothiazol-2-amine

### Example 16A

Example 16A (0.63 g) was prepared following the procedure for Example 1A, substituting (3,4-dihydro-2H-benzo[b][1,4]dioxepin-6-yl)methanamine for benzyl amine. Example 16A was used in the next step without further purification.

### Example 16B

### N-(3,4-dihydro-2H-1,5-benzodioxepin-6-ylmethyl)-6-pyridin-4-yl-1,3-benzothiazol-2-amine

The TFA salt of the title compound (0.003 g) was synthesized using the procedure from Example 1B, substituting Example 16A for Example 1A. ¹H NMR (300 MHz, CD₃OD) δ ppm 2.08-2.29 (m, 2H)4.06-4.31 (m, 4H) 4.68 (s, 2H) 6.84-6.97 (m, 2H) 6.98-7.13 (m, 1H) 7.61 (d, J=8.48 Hz, 1H) 7.93 (dd, J=8.82, 2.03 Hz, 1H) 8.22-8.39 (m, 3H) 8.73 (d, J=6.78 Hz, 2H).

### Example 17

### N-(2,3-dihydro-1,4-benzodioxin-5-ylmethyl)-6-(1H-pyrrolo[2,3-b]pyridin-4-yl)-1,3-benzothiazol-2-amine

The TFA salt of the title compound (0.003 g) was synthesized following the procedure from Example 6B substituting Example 12A for Example 2A. ¹H NMR (300 MHz, CD₃OD) δ ppm 4.17-4.43 (m, 2H) 4.68 (s, 2H) 6.74-6.87 (m, 2H) 6.92 (s, 1H) 7.02 (d, J=3.73 Hz, 1H) 7.54-7.73 (m, 3H) 7.89 (dd, J=8.48, 2.03 Hz, 1H) 8.24 (d, J=1.70 Hz, 1H) 8.38 (d, J=5.76 Hz, 1H).

### Example 18

### N-(2-ethoxybenzyl)-6-pyridin4-yl-1,3-benzothiazol-2-amine

### Example 18A

Example 18A (0.58 g) was prepared following the procedure for Example 1A, substituting (2-ethoxyphenyl)methanamine for benzyl amine. Example 18A was used in the next step without further purification.

### Example 18B

### N-(2-ethoxybenzyl)-6-pyridin4-yl-1,3-benzothiazol-2-amine

The TFA salt of the title compound (0.016 g) was synthesized using the procedure from Example 1B, substituting Example 18A for Example 1A. ¹H NMR (300 MHz, CD₃OD) δ ppm 1.39 (t, J=6.95 Hz, 3H) 4.11 (q, J=7.12 Hz, 2H) 4.70 (s, 2H) 6.84-7.05 (m, 2H) 7.21-7.39 (m, 2H) 7.61 (d, J=8.48 Hz, 1H) 7.94 (dd, J=8.82, 2.03 Hz, 1H) 8.26-8.41 (m, 3H) 8.73 (d, J=6.78 Hz, 2H).

### Example 19

### N-[2-(methylthio)benzyl]-6-pyridin-4-yl-1,3-benzothiazol-2-amine

### Example 19A

Example 19A (0.58 g) was prepared following the procedure for Example 1A, substituting (2-(methylthio)phenyl)methanamine for benzyl amine. Example 19A was used in the next step without further purification.

### Example 19B

### N-[2-(methylthio)benzyl]-6-pyridin-4-yl-1,3-benzothiazol-2-amine

The TFA salt of the title compound (0.013 g) was synthesized using the procedure from Example 1B, substituting Example 19A for Example 1A. ¹H NMR (300 MHz, CD₃OD) δ ppm 2.52 (s, 3H) 4.76 (s, 2H) 7.19 (dd, J=7.46, 1.36 Hz, 1H) 7.27-7.47 (m, 3H) 7.62 (d, J=8.48 Hz, 1H) 7.94 (dd, J=8.82, 2.03 Hz, 1H) 8.25-8.44 (m, 3H) 8.74 (d, J=7.12 Hz, 2H).

### Example 20

### N-[2-(difluoromethoxy)benzyl]-6-pyridin-4-yl-1,3-benzothiazol-2-amine

### Example 20A

Example 20A (0.42 g) was prepared following the procedure for Example 1A, substituting (2-(difluoromethoxy)phenyl)methanamine for benzyl amine. Example 20A was used in the next step without further purification.

### Example 20B

### N-[2-(difluoromethoxy)benzyl]-6-pyridin-4-yl-1,3-benzothiazol-2-amine

The TFA salt of the title compound (0.003 g) was synthesized using the procedure from Example 1B, substituting Example 20A for Example 1A. ¹H NMR (300 MHz, CD₃OD) δ ppm 6.91 (t, 1H) 7.23 (t, J=7.54 Hz, 2H) 7.23 (t, J=7.54 Hz, 1H) 7.35 (dd, J=7.34, 1.78 Hz, 1H) 7.49 (d, J=7.93 Hz, 1H) 7.61 (d, J=8.33 Hz, 1H) 7.94 (dd, J=8.73, 1.98 Hz, 1H) 8.25-8.43 (m, 3H) 8.74 (d, J=6.74 Hz, 2H) 8.74 (d, J=6.74 Hz, 2H).

### Example 21

### N-[3-(difluoromethoxy)benzyl]-6-pyridin-4-yl-1,3-benzothiazol-2-amine

### Example 21A

Example 21 A (0.48 g) was prepared following the procedure for Example 1A, substituting (3-(difluoromethoxy)phenyl)methanamine for benzyl amine. Example 21A was used in the next step without further purification.

### Example 21B

### N-[3-(difluoromethoxy)benzyl]-6-pyridin-4-yl-1,3-benzothiazol-2-amine

The TFA salt of the title compound (0.027 g) was synthesized using the procedure from Example 1B, substituting Example 21A for Example 1A. ¹H NMR (300 MHz, CD₃OD) δ ppm 4.72 (s, 2H) 6.37-7.01 (m, 1H) 6.47-7.05 (m, 1H) 7.05-7.11 (m, 1H) 7.20 (s, 1H) 7.24-7.32 (m, 1H) 7.39 (t, J=7.73 Hz, 1H) 7.62 (d, J=8.72 Hz, 1H) 7.93 (dd, J=8.53, 2.18 Hz, 1H) 8.28-8.38 (m, 3H) 8.74 (d, J=6.74 Hz, 2H).

### Example 22

### N-[3-fluoro-5-(trifluoromethyl)benzyl]-6-pyridin-4-yl-1,3-benzothiazol-2-amine

### Example 22A

6-bromo-2-chlorobenzo[d]thiazole (0.065 g, 0.262 mmol) was added to a reaction flask containing diisopropylethylamine (0.07 mL, 0.524 mmol) and (3-fluoro-5-(trifluoromethyl)phenyl)methanamine (0.237 g, 0.065 mmol). The reaction mixture was heated at 95° C. under a steady stream of N₂ for 12 hours. Methanol (1 mL) was added to the reaction mixture after cooling and the solvents were removed under vacuum. The crude product was purified via HPLC and used in the next step.

### Example 22B

### N-[3-fluoro-5-(trifluoromethyl)benzyl]-6-pyridin-4-yl-1,3-benzothiazol-2-amine

The TFA salt of the title compound (0.001 g) was synthesized using the procedure from Example 1B, substituting Example 22A for Example 1A. ¹H NMR (500 MHz, DMSO-d₆) δ ppm 4.75 (s, 2H) 7.55 (d, J=8.54 Hz, 4H) 7.80 (s, 1H) 7.96 (s, 2H) 8.32 (d, J=1.53 Hz, 1H) 8.66 (s, 2H).

### Example 23

### N-[2-(2-methylphenyl)ethyl]-6-pyridin-4-yl-1,3-benzothiazol-2-amine

### Example 23A

Example 23A was prepared following the procedure for Example 22A, substituting 2-m-tolylethanamine for (3-fluoro-5-(trifluoromethyl)phenyl)methanamine.

### Example 23B

### N-[2-(2-methylphenyl)ethyl]-6-pyridin-4-yl-1,3-benzothiazol-2-amine

The TFA salt of the title compound (0.0042 g) was synthesized using the procedure from Example 1B, substituting Example 23A for Example 1A. ¹H NMR (500 MHz, DMSO-d₆) δ ppm 2.33 (s, 3H) 2.94 (t, J=7.48 Hz, 2H) 3.60 (s, 2H) 6.79 (s, 1H) 7.03-7.27 (m, 4H) 7.54 (d, J=8.24 Hz, 1H) 7.80 (dd, J=8.54, 2.14 Hz, 1H) 8.15 (s, 1H) 8.27 (d, J=1.22 Hz, 2H) 8.65 (s, 1H).

### Example 24

### 2-[methyl(6-pyridin-4-yl-1,3-benzothiazol-2-yl)amino]-1-phenylethanol

### Example 24A

Example 24A was prepared following the procedure for Example 22A, substituting 2-(methylamino)-1-phenylethanol for (3-fluoro-5-(trifluoromethyl)phenyl)methanamine.

### Example 24B

### 2-[methyl(6-pyridin-4-yl-1,3-benzothiazol-2-yl)amino]-1-phenylethanol

The TFA salt of the title compound (0.0048 g) was synthesized using the procedure from Example 1B substituting Example 24A for Example 1A. ¹H NMR (500 MHz, DMSO-d₆) δ ppm 2.53-2.61 (m, 2H) 3.21 (s, 3H) 5.01 (d, J=5.80 Hz, 1H) 7.25-7.52 (m, 4H) 7.62 (d, J=8.54 Hz, 2H) 7.95 (dd, J=8.39, 1.98 Hz, 1H) 8.25 (d, J=6.41 Hz, 2H) 8.47 (d, J=1.83 Hz, 1H) 8.76 (d, J=6.41 Hz, 2H).

### Example 25

### N-[(1S)-1-phenylethyl]-6-pyridin-4-yl-1,3-benzothiazol-2-amine

### Example 25A

Example 25A was prepared following the procedure for Example 22A substituting (S)-1-phenylethanamine for (3-fluoro-5-(trifluoromethyl)phenyl)methanamine.

### Example 25B

### N-[(1S)-1-phenylethyl]-6-pyridin-4-yl-1,3-benzothiazol-2-amine

The TFA salt of the title compound (0.0036 g) was synthesized using the procedure from Example 1B, substituting Example 25A for Example 1A. ¹H NMR (500 MHz, DMSO-d₆) δ ppm 1.52 (d, J=7.02 Hz, 3H) 5.07 (s, 1H) 6.77 (s, 1H) 7.18-7.56 (m, 5H) 7.76 (d, J=10.68 Hz, 1H) 7.89 (s, 1H) 8.05-8.33 (m, 2H) 8.64 (s, 2H).

### Example 26

### N-[(1R)-1-phenylethyl]-6-pyridin-4-yl-1,3-benzothiazol-2-amine

### Example 26A

Example 26A was prepared following the procedure for Example 22A, substituting (R)-1-phenylethanamine for (3-fluoro-5-(trifluoromethyl)phenyl)methanamine.

### Example 26B

### N-[(1R)-1-phenylethyl]-6-pyridin-4-yl-1,3-benzothiazol-2-amine

The TFA salt of the title compound (0.126 g) was synthesized using the procedure from Example 1B, substituting Example 26A for Example 1A. ¹H NMR (500 MHz, DMSO-d₆) δ ppm 1.57 (d, J=7.02 Hz, 3H) 5.10 (s, 1H) 6.77 (s, 1H) 7.18-7.56 (m, 5H)7.76 (d, J=10.68 Hz 1H) 7.89 (s, 1H) 8.05-8.33 (m, 2H) 8.61 (s, 2H).

### Example 27

### N-(2-phenoxyethyl)-6-pyridin-4-yl-1,3-benzothiazol-2-amine

### Example 27A

Example 27A was prepared following the procedure for Example 22A, substituting 2-phenoxyethanamine for (3-fluoro-5-(trifluoromethyl)phenyl)methanamine.

### Example 27B

### N-(2-phenoxyethyl)-6-pyridin-4-yl-1,3-benzothiazol-2-amine

The TFA salt of the title compound (0.0015 g) was synthesized using the procedure from Example 1B, substituting Example 27A for Example 1A. ¹H NMR (500 MHz, DMSO-d₆) δ ppm 1.44-1.62 (m, 3H) 5.07 (s, 1H) 7.03 (s, 1H) 7.17-7.58 (m, 5H) 7.81 (s, 1H) 8.05 (s, 2H) 8.32 (d, J=1.83 Hz, 1H) 8.69 (d, J=6.10 Hz, 2H).

### Example 28

### N-[(1R)-1-(1-naphthyl)ethyl]-6-pyridin-4-yl-1,3-benzothiazol-2-amine

### Example 28A

Example 28A was following the procedure for Example 22A, substituting (R)-1-(naphthalen-2-yl)ethanamine for (3-fluoro-5-(trifluoromethyl)phenyl)methanamine.

### Example 28B

### N-[(1R)-1-(1-naphthyl)ethyl]-6-pyridin-4-yl-1,3-benzothiazol-2-amine

The TFA salt of the title compound (0.0068 g) was synthesized using the procedure from Example 1B, substituting Example 28A for Example 1A. ¹H NMR (500 MHz, DMSO-d₆) δ ppm 1.68 (d, J=6.71 Hz, 3H) 5.90 (s, 1H) 7.47-7.67 (m, 5H) 7.83-7.94 (m, 2H) 7.99 (d, J=7.32 Hz, 1H) 8.20-8.31 (m, 3H) 8.41 (d, J=2.14 Hz, 1H) 8.77 (d, J=6.71 Hz, 2H).

### Example 29

### N-[(1S)-1-(1-naphthyl)ethyl]-6-pyridin-4-yl-1,3-benzothiazol-2-amine

### Example 29A

Example 29A was prepared following the procedure for Example 22A, substituting (S)-1-(naphthalen-1-yl)ethanamine for (3-fluoro-5-(trifluoromethyl)phenyl)methanamine.

### Example 29B

### N-[(1S)-I -(1-naphthyl)ethyl]-6-pyridin-4-yl-1,3-benzothiazol-2-amine

The TFA salt of the title compound (0.0058 g) was synthesized using the procedure from Example 1B, substituting Example 29A for Example 1A. ¹H NMR (500 MHz, DMSO-d₆) δ ppm 1.68 (d, J=6.71 Hz, 3H) 5.90 (s, 1H) 7.47-7.67 (m, 5H) 7.83-7.94 (m, 2H) 7.99 (d, J=7.32 Hz, 1H) 8.20-8.31 (m, 3H) 8.41 (d, J=2.14 Hz, 1H) 8.77 (d, J=6.71 Hz, 2H).

### Example 30

### N-[(1R)-1-phenylpropyl]-6-pyridin-4-yl-1,3-benzothiazol-2-amine

### Example 30A

Example 30A (0.45 g) was prepared following the procedure for Example 22A, substituting (R)-1-phenylpropan-1-amine for (3-fluoro-5-(trifluoromethyl)phenyl)methanamine. Example 30A was used in the next step without further purification.

### Example 30B

### N-[(1R)-1-phenylpropyl]-6-pyridin-4-yl-1,3-benzothiazol-2-amine

The TFA salt of the title compound (0.132 g) was synthesized using the procedure from Example 1B, substituting Example 30A for Example 1A. ¹H NMR (300 MHz, CD₃OD) δ ppm 1.01 (t, J=7.46 Hz, 3H) 1.80-2.11 (m, 2H) 4.78-4.92 (m, 1H) 7.09-7.48 (m, 5H) 7.57 (d, J=8.48 Hz, 1H) 7.91 (dd, J=8.48, 2.03 Hz, 1H) 8.20-8.40 (m, 3H) 8.73 (d, J=7.12 Hz, 2H).

### Example 31

### N-(3-fluorobenzyl)-6-(pyridin-4-yl)benzo[d]thiazol-2-amine

### Example 31A

Example 31A (0.33 g) was prepared following the procedure for Example 1A substituting (3-fluorophenyl)methanamine for benzyl amine. Example 31A was used in the next step without further purification.

### Example 31B

### N-(3-fluorobenzyl)-6-(pyridin-4-yl)benzo[d]thiazol-2-amine

The TFA salt of the title compound (0.002 g) was synthesized using the procedure from Example 1B, substituting Example 31A for Example 1A. ¹H NMR (300 MHz, CD₃OD) δ ppm 4.67-4.75 (m, 2H) 6.95-7.08 (m, 1H) 7.10-7.29 (m, 2H) 7.32-7.43 (m, 1H) 7.62 (d, J=8.72 Hz, 1H) 7.94 (dd, J=8.53, 2.18 Hz, 1H) 8.35 (dd, J=4.76, 2.38 Hz, 3H) 8.74 (d, J=7.14 Hz, 2H).

### Example 32

### N-[(1R)-2,3-dihydro-1H-inden-1-yl]-6-pyridin-4-yl-1,3-benzothiazol-2-amine

### Example 32A

Example 32A (0.65 g) was synthesized following the procedure for Example 1A substituting (R)-2,3-dihydro-1H-inden-1-amine for benzyl amine. Example 32A was used in the next step without further purification.

### Example 32B

### N-[(1R)-2,3-dihydro-1H-inden-1-yl]-6-pyridin-4-yl-1,3-benzothiazol-2-amine

The TFA salt of the title compound (0.224 g) was synthesized following the procedure from Example 1B, substituting Example 32A for Example 1A. ¹H NMR (300 MHz, CD₃OD) δ ppm 1.94-2.15 (m, 1H) 2.60-2.77 (m, 1H) 2.85-3.18 (m, 2H) 5.56 (t, J=7.12 Hz, 1H) 7.15-7.44 (m, 4H) 7.65 (d, J=8.82 Hz, 1H) 7.97 (dd, J=8.48, 2.03 Hz, 1H) 8.31-8.42 (m, 3H) 8.75 (d J=6.78 Hz, 2H).

### Example 33

### N-[(1S)-2,3-dihydro-1H-inden-1-yl]-6-pyridin-4-yl-1,3-benzothiazol-2-amine

### Example 33A

Example 33A (0.71 g) was synthesized following the procedure for Example 1A, substituting (S)-2,3-dihydro-1H-inden-1-amine for benzyl amine. Example 33A was used in the next step without further purification.

### Example 33B

### N-[(1S)-2,3-dihydro-1H-inden-1-yl]-6-pyridin-4-yl-1,3-benzothiazol-2-amine

The TFA salt of the title compound (0.305 g) was synthesized following the procedure from Example 1B, substituting Example 33A for Example 1A. ¹H NMR (300 MHz, CD₃OD) δ ppm 1.94-2.13 (m, 2H) 2.61-2.77 (m, 1H) 2.87-3.02 (m, 1H) 3.06 (dd, J=8.48, 4.41 Hz, 1H) 5.56 (t, J=6.95 Hz, 1H) 7.11-7.35 (m, 3H) 7.38 (d, J=7.12 Hz, 1H) 7.65 (d, J=8.48 Hz, 1H) 7.98 (dd, J=8.48, 2.03 Hz, 1H) 8.30-8.44 (m, 3H) 8.75 (d, J=7.12 Hz, 2H).

### 1021025 Example 34

### 6-pyridin-4-yl-N-[3-(trimethylsilyl)propyl]-1,3-benzothiazol-2-amine

### Example 34A

Example 34A was synthesized following the procedure for Example 1A, substituting 3-(trimethylsilyl)propan-1-amine for benzyl amine.

### Example 34B

### 6-pyridin-4-yl-N-[3-(trimethylsilyl)propyl]-1,3-benzothiazol-2-amine

The TFA salt of the title compound (0.024 g) was synthesized following the procedure from Example 1B, substituting Example 34A for Example 1A. ¹H NMR (300 MHz, METHANOL-d₄) δ ppm 8.81 (d, J=6.4, 2H), 7.94 (d, J=1.5, 1H), 7.76 (ddd, J=5.0, 13.9, 22.8, 4H), 3.41 (t, J=7.0, 2H), 1.91-1.71 (m, 2H), 0.70-0.53 (m, 2H).

### Example 35

### N-benzyl-6-(1H-indazol-5-yl)-1,3-benzothiazol-2-amine

Example 35 (0.019 g) was synthesized following the procedure from Example 6B, substituting Example 1A for Example 2A and substituting 1-(tert-butoxycarbonyl)-1H-indazol-5-ylboronic acid for Example 6A. ¹H NMR (300 MHz, METHANOL-d₄) δ ppm 8.61 (s, 1H), 8.10 (d, J=0.8, 1H), 8.00 (dd, J=1.3, 11.8, 2H), 7.70-7.52 (m, 3H) 7.48-7.23 (m, 6H), 4.63 (d, J=5.7, 2H).

### Example 36

### 6-(1H-indazol-5-yl)-N-(3-phenylpropyl)-1,3-benzothiazol-2-amine

Example 36 (0.019 g) was synthesized following the procedure from Example 35, substituting Example 5A for Example 1A. ¹H NMR (300 MHz, DMSO-d₆) δ 8.51 (s, 1H), 8.11 (d, J=0.9, 1H), 8.05 (d, J=1.8, 1H), 7.99 (d, J=0.9, 1H), 7.72-7.53 (m, 3H), 7.46 (d, J =8.4, 1H), 7.26 (ddt, J=7.0, 8.5, 13.7, 5H), 3.41 (d, J=5.3, 3H), 2.78-2.64 (m, 2H), 1.94 (dd, J=7.4, 14.8, 2H).

### Example 37

### N-[(1R)-1-(2-naphthyl)ethyl]-6-pyridin-4-yl-1,3-benzothiazol-2-amine

### Example 37A

Example 37A (0.41 g) was synthesized following the procedure for Example 1A, substituting (R)-1-(naphthalen-2-yl)ethanamine for benzyl amine. The crude material was used in the next step without further purification.

### Example 37B

### N-[(1R)-1-(2-naphthyl)ethyl]-6-pyridin-4-yl-1,3-benzothiazol-2-amine

The TFA salt of the title compound (0.025 g) was synthesized following the procedure from Example 1B, substituting Example 37A for Example 1A. ¹H NMR (300 MHz, METHANOL-d₄) δ ppm 8.73 (d, J=7.0, 2H), 8.32 (dd, J=4.5, 9.1, 3H), 8.21 (d, J=8.5, 1H), 7.99-7.75 (m, 3H), 7.71-7.39 (m, 5H), 5.93 (d, J=6.5, 1H), 1.78 (d, J=6.8, 3H).

### Example 38

### N-(2,3-dimethoxybenzyl)-6-pyridin-4-yl-1,3-benzothiazol-2-amine

### Example 38A

Example 38A was synthesized following the procedure for Example 1A, substituting (2,3-dimethoxyphenyl)methanamine for benzyl amine.

### Example 38B

### N-(2,3-dimethoxybenzyl)-6-pyridin-4-yl-1,3-benzothiazol-2-amine

The TFA salt of the title compound (0.165 g) was synthesized following the procedure from Example 1B, substituting Example 38A for Example 1A. ¹H NMR (300 MHz, METHANOL-d₄) δ ppm 8.76-8.70 (m, 2H), 8.35-8.29 (m, 3H), 7.93 (dd, J=2.1, 8.5, 1H), 7.61 (d, J=8.5, 1H), 7.25 (t, J=8.1, 1H), 6.98-6.92 (m, 2H), 6.86-6.79 (m, 1H), 1.30 (s, 3H), 1.28 (s, 4H).

### Example 39

### N-(2,5-dimethoxybenzyl)-6-pyridin-4-yl-1,3-benzothiazol-2-amine

### Example 39A

Example 39A (0.38 g) was synthesized following the procedure for Example 1A, substituting (2,5-dimethoxyphenyl)methanamine for benzyl amine. The crude material was used in the next step without further purification.

### Example 39B

### N-(2,5-dimethoxybenzyl)-6-pyridin-4-yl-1,3-benzothiazol-2-amine

The TFA salt of the title compound (0.076 g) was synthesized following the procedure from Example 1B, substituting Example 39A for Example 1A. ¹H NMR (300 MHz, METHANOL-d₄) δ ppm 8.79-8.73 (m, 2H), 8.38-8.32 (m, 3H), 7.96 (dd, J=2.0, 8.6, 1H), 7.63 (d, J=8.5, 1H), 6.96 (d, J=1.8, 1H), 6.94 (d, J=7.4, 1H), 6.85 (dd, J=3.0, 8.9. 1H), 4.66 (s, 2H), 3.84 (s, 3H), 3.73 (s, 3H).

### Example 40

### (2R)-2-phenyl-2-[(6-pyridin-4-yl-1,3-benzothiazol-2-yl)amino]ethanol

### Example 40A

Example 40A was synthesized following the procedure for Example 1A, substituting (R)-2-amino-2-phenylethanol for benzyl amine.

### Example 40B

### (2R)-2-phenyl-2-[(6-pyridin-4-yl-1,3-benzothiazol-2-yl)amino]ethanol

The TFA salt of the title compound (0.017 g) was synthesized following the procedure from Example 1B, substituting Example 40A for Example 1A. ¹H NMR (300 MHz, METHANOL-d₄) δ ppm 8.78-8.71 (m, 2H), 8.38-8.31 (m, 3H), 7.93 (dd, J=2.1, 8.6, 1H), 7.59 (d, J=8.6, 1H), 7.49-7.42 (m, 2H), 7.42-7.33 (m, 2H), 7.34-7.24 (m, 1H), 5.08 (dd, J=5.0, 7.4, 1H), 3.96-3.79 (m, 2H).

### Example 41

### N-(3-isopropoxybenzyl)-6-pyridin-4-yl-1,3-benzothiazol-2-amine

### Example 41A

Example 41A was synthesized following the procedure for Example 1A, substituting (3-isopropoxyphenyl)methanamine for benzyl amine.

### Example 41B

### N-(3-isopropoxybenzyl)-6-pyridin-4-yl-1,3-benzothiazol-2-amine

The TFA salt of the title compound (0.017 g) was synthesized following the procedure from Example 1B, substituting Example 41A for Example 1A. ¹H NMR (300 MHz, METHANOL-d₄) δ ppm 8.78-8.71 (m, 2H), 8.38-8.32 (m, 3H), 7.95 (dd, J=2.0, 8.6, 1H), 7.62 (d, J=8.5, 1H), 7.11-6.94 (m, 3H), 3.95-3.83 (m, 6H).

### Example 42

### N-[(2,2-dimethyl-2,3-dihydro-1-benzofuran-7-yl)methyl]-6-pyridin-4-yl-1,3-benzothiazol-2-amine

### Example 42A

Example 42A was synthesized following the procedure for Example 1A, substituting (2,2-dimethyl-2,3-dihydrobenzofuran-7-yl)methanamine for benzyl amine.

### Example 42B

### N-[(2,2-dimethyl-2,3-dihydro-1-benzofuran-7-yl)methyl]-6-pyridin-4-yl-1,3-benzothiazol-2-amine

The TFA salt of the title compound (0.107 g) was synthesized following the procedure from Example 1B, substituting Example 42A for Example 1A. ¹H NMR (300 MHz, DMSO-d₆) δ ppm 8.83 (d, J=6.7, 2H), 8.46 (s, 1H), 8.27 (s, 2H), 7.92 (s, 1H), 7.55 (d, J=8.5, 1H), 7.11 (d, J=7.8, 2H), 6.80 (d, J=7.5, 1H), 4.52 (d, J=5.4, 2H), 3.03 (s, 2H), 1.45 (s, 6H), −0.00 (s, 4H).

### Example 43

### 2-(4-methyl-2-phenylpiperazin-1-yl)-6-pyridin-4-yl-1,3-benzothiazole

### Example 43A

Example 43A was synthesized following the procedure for Example 1A, substituting 1-methyl-3-phenylpiperazine for benzyl amine.

### Example 43B

### 2-(4-methyl-2-phenylpiperazin-1-yl)-6-pyridin-4-yl-1,3-benzothiazole

The TFA salt of the title compound (0.119 g) was synthesized following the procedure from Example 1B, substituting Example 43A for Example 1A.

### Example 44

### N-(2,3-dichlorobenzyl)-6-pyridin-4-yl-1,3-benzothiazol-2-amine

### Example 44A

Example 44A was synthesized following the procedure for Example 1A, substituting (2,3-dichlorophenyl)methanamine for benzyl amine.

### Example 44B

### N-(2,3-dichlorobenzyl)-6-pyridin-4-yl-1,3-benzothiazol-2-amine

The TFA salt of the title compound (0.147 g) was synthesized following the procedure from Example 1B, substituting Example 44A for Example 1A. ¹H NMR (300 MHz, DMSO-d₆) δ ppm 8.98-8.90 (m, 1H), 8.80-8.73 (m, 2H), 8.41 (d, J=2.0, 1H), 8.14-8.08 (m, 2H), 7.87 (dd, J=2.0, 8.5, 1H), 7.64-7.34 (m, 4H), 4.76 (d, J=5.6, 2H).

### Example 45

### (3R)-3-phenyl-3-[(6-pyridin-4-yl-1,3-benzothiazol-2-yl)amino]propan-1-ol

### Example 45A

Example 45A was synthesized following the procedure for Example 1A, substituting (R)-3-amino-3-phenylpropan-1-ol for benzyl amine.

### Example 45B

### (3R)-3-phenyl-3-[(6-pyridin-4-yl-1,3-benzothiazol-2-yl)amino]propan-1-ol

The TFA salt of the title compound (0.036 g) was synthesized following the procedure from Example 1B, substituting Example 45A for Example 1A. ¹H NMR (300 MHz, METHANOL-d₄) δ ppm 8.78-8.71 (m, 2H), 8.38-8.30 (m, 3H), 7.97-7.89 (m, 1H), 7.59 (d, J=8.6, 1H), 7.48-7.23 (m, 6H), 5.24-5.08 (m, 1H), 3.75-3.57 (m, 2H).

### Example 46

### N-(2-fluorobenzyl)-6-pyridin-4-yl-1,3-benzothiazol-2-amine

### Example 46A

Example 46A was synthesized following the procedure for Example 1A, substituting (2-fluorophenyl)methanamine for benzyl amine.

### Example 46B

### N-(2-fluorobenzyl)-6-pyridin-4-yl-1,3-benzothiazol-2-amine

The TFA salt of the title compound (0.084 g) was synthesized following the procedure from Example 1B, substituting Example 46A for Example 1A. ¹H NMR (300 MHz, METHANOL-d₄) δ ppm 8.74 (d, J=6.6, 2H), 8.39-8.32 (m, 3H), 7.94 (dd, J=2.0, 8.6, 1H), 7.62 (d, J=8.6, 1H), 7.47 (t, J=7.6, 1H), 7.33 (dt, J=3.9, 13.2, 1H), 7.22-7.08 (m, 2H), 4.76 (s, 2H).

### Example 47

### N-(2,3-difluorobenzyl)-6-pyridin-4-yl-1,3-benzothiazol-2-amine

### Example 47A

Example 47A was synthesized following the procedure for Example 1A substituting (2,3-difluorophenyl)methanamine for benzyl amine.

### Example 47B

### N-(2,3-difluorobenzyl)-6-pyridin-4-yl-1,3-benzothiazol-2-amine

The TFA salt of the title compound (0.098 g) was synthesized following the procedure from Example 1B, substituting Example 47A for Example 1A. ¹NMR (300 MHz, METHANOL-d₄) δ ppm 8.74 (d, J=7.0, 2H), 8.35 (dd, J=2.4, 4.6, 3H), 7.94 (dd, J=2.1, 8.6, 1H), 7.63 (d, J=8.5, 1H), 7.32-7.11 (m, 3H), 4.80 (s, 2H).

### Example 48

### N-(2,3-dimethylbenzyl)-6-pyridin-4-yl-1,3-benzothiazol-2-amine

### Example 48A

Example 48A was synthesized following the procedure for Example 1A, substituting (2,3-dimethylphenyl)methanamine for benzyl amine.

### Example 48B

### N-(2,3-dimethylbenzyl)-6-pyridin-4-yl-1,3-benzothiazol-2-amine

The TFA salt of the title compound (0.012 g) was synthesized following the procedure from Example 1B, substituting Example 48A for Example 1A. ¹NMR (300 MHz, METHANOL-d₄) δ ppm 8.74 (d, J=6.9, 2H), 8.39-8.32 (m, 3H), 7.95 (dd, J=2.1, 8.5, 1H), 7.62 (d, J=8.7, 1H), 7.19 (d, J=7.8, 1H), 7.16-7.03 (m, 2H), 4.70 (s, 2H), 2.33 (t, J=13.6, 6H).

### Example 49

### N-[1-(2,3-dihydro-1,4-benzodioxin-5-yl)ethyl]-6-pyridin-4-yl-1,3-benzothiazol-2-amine

### Example 49A

Example 49A was synthesized following the procedure for Example 1A, substituting 1-(2,3-dihydrobenzo[b][1,4]dioxin-5-yl)ethanamine for benzyl amine.

### Example 49B

### N-[1-(2,3-dihydro-1,4-benzodioxin-5-yl)ethyl]-6-pyridin-4-yl-1,3-benzothiazol-2-amine

The TFA salt of the title compound (0.088 g) was synthesized following the procedure from Example 1B, substituting Example 49A for Example 1A. ¹H NMR (300 MHz, METHANOL-d₄) δ ppm 8.74 (d, J=7.0. 2H), 8.41-8.27 (m, 3H), 7.94 (dd, J=2.1, 8.6, 1H), 7.59 (d, J=8.6, 1H), 6.96-6.71 (m, 3H), 5.05-4.87 (m, 1H), 4.58 (s, 0H), 4.29-4.09 (m, 4H), 1.57 (d, J=6.8, 3H), 1.38 (d, J=6.4, 0H).

### Example 50

### N-[1-(2-chlorophenyl)ethyl]-6-pyridin-4-yl-1,3-benzothiazol-2-amine

### Example 50A

Example 50A was synthesized following the procedure for Example 1A, substituting 1-(2-chlorophenyl)ethanamine for benzyl amine.

### Example 50B

### N-[1-(2-chlorophenyl)ethyl]-6-pyridin-4-yl-1,3-benzothiazol-2-amine

The TFA salt of the title compound (0.088 g) was synthesized following the procedure from Example 1B, substituting Example 50A for Example 1A. ¹H NMR (300 MHz, METHANOL-d₄) δ ppm 8.73 (d, J=6.8, 2H), 8.33 (d, J=6.7, 3H), 7.90 (dd, J=2.1, 8.6, 1H), 7.58 (d. J=8.5, 1H), 7.50 (dd, J=1.8, 7.6, 1H), 7.42 (dd, J=1.8, 7.4, 1H), 7.35-7.21 (m, 2H), 5.43 (q, J=6.8, 1H), 1.61 (d, J=6.8, 3H).

### Example 51

### (2S)-2-phenyl-2-[(6-pyridin-4-yl-1,3-benzothiazol-2-yl)amino]ethanol

### Example 51A

Example 51A was synthesized following the procedure for Example 1A, substituting (S)-2-amino-2-phenylethanol for benzyl amine.

### Example 51B

### (2S)-2-phenyl-2-[(6-pyridin-4-yl-1,3-benzothiazol-2-yl)amino]ethanol

The TFA salt of the title compound (0.080 g) was synthesized following the procedure from Example 1B, substituting Example 51A for Example 1A. ¹H NMR (300 MHz, METHANOL-d₄) δ ppm 8.75 (d, J=6.5, 2H), 8.35 (d, J=6.4, 3H), 7.93 (dd, J=1.8, 8.7, 1H), 7.59 (d, J=8.6, 1H), 7.48-7.27 (m, 5H), 5.12-5.04 (m, 1H), 3.96-3.79 (m, 2H).

### Example 52

### 3-(3-methoxyphenyl)-3-[(6-pyridin-4-yl-1,3-benzothiazol-2-yl)amino]propan-1-ol

### Example 52A

Example 52A was synthesized following the procedure for Example 1A, substituting 3-amino-3-(3-methoxyphenyl)propan-1-ol for benzyl amine.

### Example 52B

### 3-(3-methoxyphenyl)-3-[(6-pyridin-4-yl-1,3-benzothiazol-2-yl)amino]propan-1-ol

The TFA salt of the title compound (0.005 g) was synthesized following the procedure from Example 1B, substituting Example 52A for Example 1A. ¹H NMR (300 MHz, METHANOL-d₄) δ ppm 8.74 (d, J=6.9, 2H), 8.33 (dd, J=2.4, 4.6, 3H), 7.94 (dd, J=2.0, 8.6, 1H), 7.60 (d, J=8.6, 1H), 7.24 (s, 1H), 6.97 (s, 2H), 6.89-6.71 (m, 1H), 3.79 (d. J=8.3, 3H), 3.59 (d, J=6.5, 2H), 2.09 (d, J=6.8, 2H), 1.29 (d, J=2.5, 1H).

### Example 53

### N-(3-methylbenzyl)-6-pyridin-4-yl-1,3-benzothiazol-2-amine

### Example 53A

Example 53A was synthesized following the procedure for Example 1A, substituting m-tolylmethanamine for benzyl amine.

### Example 53B

### N-(3-methylbenzyl)-6-pyridin-4-yl-1,3-benzothiazol-2-amine

The TFA salt of the title compound (0.25 g) was synthesized following the procedure from Example 1B, substituting Example 53A for Example 1A. ¹H NMR (300 MHz, METHANOL-d₄) δ ppm 8.78-8.72 (m, 2H), 8.35 (dd, J=1.6, 5.5, 3H), 7.95 (dd, J=2.1, 8.7, 1H), 7.62 (d, J=8.6, 1H), 7.29-7.15 (m, 3H), 7.15-7.08 (m, 1H), 4.66 (s, 2H), 2.34 (s, 3H).

### Example 54

### (1S,2R)-1-[(6-pyridin-4-yl-1,3-benzothiazol-2-yl)amino]indan-2-ol

### Example 54A

Example 54A was synthesized following the procedure for Example 1A, substituting (1S,2R)-1-amino-2,3-dihydro-1H-inden-2-ol for benzyl amine.

### Example 54B

### (1S,2R)-1-[(6-pyridin-4-yl-1,3-benzothiazol-2-yl)amino]indan-2-ol

The TFA salt of the title compound (0.1 g) was synthesized following the procedure from Example 1B, substituting Example 54A for Example 1A. ¹H NMR (300 MHz, METHANOL-d₄) δ ppm 8.94-8.70 (m, 2H), 8.50-8.31 (m, 3H), 7.98 (dd, J=2.1, 8.6, 1H), 7.65 (d, J=8.5, 1H), 7.29 (ddd, J=5.9, 13.8, 15.3, 4H), 5.52 (d, J=5.0, 1H), 4.77 (dt, J=2.3, 5.2, 1H), 3.25-3.14 (m, 1H), 3.00 (dd, J=2.3, 16.4, 1H).

### Example 55

### N-[3-(hydroxyamino)-1-(3-methoxyphenyl)propyl]-6-pyridin-4-yl-1,3-benzothiazol-2-amine

### Example 55A

Example 55A was synthesized following the procedure for Example 1A, substituting 3-(hydroxyamino)-1-(3-methoxyphenyl)propan-1-amine for benzyl amine.

### Example 55B

### N-[3-(hydroxyamino)-1-(3-methoxyphenyl)propyl]-6-pyridin-4-yl-1,3-benzothiazol-2-amine

The TFA salt of the title compound (0.066 g) was synthesized following the procedure from Example 1B, substituting Example 55A for Example 1A. ¹H NMR (300 MHz, METHANOL-d₄) δ ppm 8.78 (d, J=7.0, 2H), 8.44-8.30 (m, 3H), 7.99 (dd, J=2.0, 8.6, 1H), 7.63 (d, J=8.5, 1H), 7.36-7.18 (m, 1H), 7.03-6.78 (m, 3H), 4.31 (dd, J=5.0, 8.3, 1H), 3.66-3.54 (m, 2H), 3.23 (s, 3H), 2.25-1.92 (m, 2H).

### Example 56

### (1R,2S)-1-[(6-pyridin-4-yl-1,3-benzothiazol-2-yl)amino]indan-2-ol

### Example 56A

Example 56A was synthesized following the procedure for Example 1A, substituting (1R,2S)-1-amino-2,3-dihydro-1H-inden-2-ol for benzyl amine.

### Example 56B

### (1R,2S)-1-[(6-pyridin-4-yl-1,3-benzothiazol-2-yl)amino]indan-2-ol

The TFA salt of the title compound (0.063 g) was synthesized following the procedure from Example 1B, substituting Example 56A for Example 1A. ¹H NMR (300 MHz, METHANOL-d₄) δ ppm 8.76 (d, J=6.9, 2H), 8.47-8.31 (m, 3H), 7.98 (dd, J=2.0, 8.5, 1H), 7.65 (d, J=8.5, 1H), 7.46-7.12 (m, 4H), 5.53 (d, J=5.0, 1H), 4.77-4.69 (m, 1H), 3.20 (d, J=5.3, 1H), 3.00 (dd, J=2.3, 16.4, 1H).

### Example 57

### N-[(1R)-1-(2,3-dihydro-1,4-benzodioxin-5-yl)ethyl]-6-pyridin-4-yl-1,3-benzothiazol-2-amine

### Example 58

### N-[(1S)-1-(2,3-dihydro-1,4-benzodioxin-5-yl)ethyl]-6-pyridin-4-yl-1,3-benzothiazol-2-amine

The TFA salt of Examples 57(0.002 g) and 58(0.003 g) were isolated from chiral separation of Example 49(0.010 g) using a preparative HPLC on a 250 mm ChiralPak AD-H 4.6 mm i.d. column with a 5 μm particle size. An isocratic gradient of hexanes/ethanol/methanol/diethylamine (34.6/40/25/0.1) was used, at a flow rate of 1 mL/min, a pressure of 100 bar and a temeperature of 25° C. The sample was injected as solutions in methanol. Fractions (with retention times of 11.96 min and 17.43 min respectively)were collected based upon UV signal threshold and selected fractions subsequently analyzed by flow injection analysis mass spectrometry using positive APCI ionization on a Finnigan LCQ using 70:30 methanol:10 mM NH₄OH(aq) at a flow rate of 0.8 mL/min. Loop-injection mass spectra were acquired using a Finnigan LCQ running LCQ Navigator 1.2 software and a Gilson 215 liquid handler for fraction injection controlled by an Abbott developed Visual Basic application

### Example 59

### N-(3-chlorobenzyl)-6-pyridin-4-yl-1,3-benzothiazol-2-amine

### Example 59A

Example 59A was synthesized following the procedure for Example 1A, substituting (3-chlorophenyl)methanamine for benzyl amine.

### Example 59B

### N-(3-chlorobenzyl)-6-pyridin-4-yl-1,3-benzothiazol-2-amine

The TFA salt of the title compound (0.163 g) was synthesized following the procedure from Example 1B, substituting Example 59A for Example 1A. ¹H NMR (300 MHz, METHANOL-d₄) δ ppm 8.73 (d, J=7.0, 2H), 8.32 (dd, J=4.5, 6.1, 3H), 7.92 (dd, J=2.1, 8.6, 1H), 7.61 (d, J=8.5, 1H), 7.43 (dd, J=0.7, 1.3, 1H), 7.38-7.27 (m, 3H), 4.70 (s, 2H).

### Example 60

### N-[1-(3-fluorophenyl)ethyl]-6-pyridin-4-yl-1,3-benzothiazol-2-amine

### Example 60A

Example 60A was synthesized following the procedure for Example 1A, substituting 1-(3-fluorophenyl)ethanamine for benzyl amine.

### Example 60B

### N-[1-(3-fluorophenyl)ethyl]-6-pyridin-4-yl-1,3-benzothiazol-2-amine

The TFA salt of the title compound (0.146 g) was synthesized following the procedure from Example 1B, substituting Example 60A for Example 1A. ¹H NMR (300 MHz, METHANOL-d₄) δ ppm 8.74 (d, J=7.0, 2H), 8.34 (t, J=4.7, 3H), 7.93 (dd, J=2.1, 8.6, 1H), 7.58 (d, J=8.6, 1H), 7.37 (td, J=5.9, 7.9, 1H), 7.25 (d, J=7.7, 1H), 7.20-7.12 (m, 1H), 7.04-6.94 (m, 1H), 5.13 (q, J=6.9, 1H), 2.65 (s, 1H), 1.62 (t, J=5.5, 3H).

### Example 61

### (3R)-3-[(6-pyridin-4-yl-1,3-benzothiazol-2-yl)amino]indan-5-ol

### Example 61A

Example 61A was synthesized following the procedure for Example 1A, substituting (R)-3-amino-2,3-dihydro-1H-inden-5-ol for benzyl amine.

### Example 61B

### (3R)-3-[(6-pyridin-4-yl-1,3-benzothiazol-2-yl)amino]indan-5-ol

The TFA salt of the title compound (0.135 g) was synthesized following the procedure from Example 1B, substituting Example 61A for Example 1A. ¹H NMR (300 MHz, METHANOL-d₄) δ ppm 8.75 (d, J=7.0, 2H), 8.41-8.29 (m, 3H), 7.97 (dd, J=2.1, 8.5, 1H), 7.64 (d, J=8.5, 1H), 7.09 (d, J=8.2, 1H), 6.81 (s, 1H), 6.70 (dd, J=2.2, 8.2, 1H), 5.48 (s, 1H), 2.82 (dd, J=36.9, 44.7, 3H), 2.10-1.94 (m, 1H).

### Example 62

### N-(3-fluorobenzyl)-6-(3-fluoropyridin-4-yl)-1,3-benzothiazol-2-amine

The TFA salt of the title compound (0.039 g) was synthesized following the procedure from Example 1B, substituting Example 31A for Example 1A and substituting 3-fluoropyridin-4-ylboronic acid for 4-(4,4,5,5-tetramethyl-1,3,2-dioxaborolan-2-yl)pyridine.

¹H NMR (300 MHz, METHANOL-d₄) δ ppm 8.59 (d, J=3.3, 1H), 8.47 (d, J=5.2, 1H), 8.05 (s, 1H), 7.78-7.65 (m, 2H), 7.58 (d, J=8.5, 1H), 7.46-7.33 (m, 1H), 7.24 (d, J=8.0, 1H), 7.17 (d, J=9.8, 1H), 7.04 (t, J=8.2, 1H), 4.71 (s, 2H).

### Example 63

### (1R,2R)-1-[(6-pyridin-4-yl-1,3-benzothiazol-2-yl)amino]indan-2-ol

### Example 63A

Example 63A was synthesized following the procedure for Example 1A, substituting (1R,2R)-1-amino-2,3-dihydro-1H-inden-2-ol for benzyl amine.

### Example 63B

### (1R,2R)-1-[(6-pyridin-4-yl-1,3-benzothiazol-2-yl)amino]indan-2-ol

The TFA salt of the title compound (0.063 g) was synthesized following the procedure from Example 1B, substituting Example 63A for Example 1A. ¹H NMR (300 MHz, METHANOL-d₄) δ ppm 8.80-8.73 (m, 2H), 8.39 (dd, J=2.4, 4.7, 3H), 7.98 (dd, J=2.1, 8.6, 1H), 7.65 (d J=8.6, 1H), 7.36-7.19 (m, 4H), 5.31 (d, J=5.9, 1H), 4.54-4.43 (m, 1H), 3.44-3.32 (m, 1H), 2.90 (dd, J=6.9, 15.8, 1H).

### Example 64

### (1S,2S)-1-[(6-pyridin-4-yl-1,3-benzothiazol-2-yl)amino]indan-2-ol

### Example 64A

Example 64A was synthesized following the procedure for Example 1A, substituting (1S,2S)-1-amino-2,3-dihydro-1H-inden-2-ol for benzyl amine.

### Example 64B

### (1S,2S)-1-[(6-pyridin-4-yl-1,3-benzothiazol-2-yl)amino]indan-2-ol

The TFA salt of the title compound (0.078 g) was synthesized following the procedure from Example 1B, substituting Example 64A for Example 1A. ¹H NMR (300 MHz, METHANOL-d₄) δ ppm 8.80-8.74 (m, 2H), 8.39 (dd, J=2.3, 4.7, 3H), 7.99 (dd, J=2.0, 8.5, 1H), 7.66 (d, J=8.5, 1H), 7.29 (ddd, J=5.3, 6.3, 9.0, 4H), 5.31 (d, J=6.0, 1H), 4.54-4.43 (m, 1H), 3.34 (d, J=7.1, 1H), 2.90 (dd, J=6.9, 15.7, 1H).

### Example 65

### N-(3-fluorobenzyl)-6-(2-fluoropyridin-4-yl)-1,3-benzothiazol-2-amine

The TFA salt of the title compound (0.176 g) was synthesized following the procedure from Example 1B, substituting Example 31A for Example 1A and substituting 2-fluoropyridin-4-ylboronic acid for 4-(4,4,5,5-tetramethyl-1,3,2-dioxaborolan-2-yl)pyridine.

¹H NMR (300 MHz, METHANOL-d₄) δ ppm 8.21 (d, J=4.9, 1H), 8.10 (s, 1H), 7.74 (d, J=7.4, 1H), 7.59 (d, J=10.2, 2H), 7.37 (s, 2H), 7.23 (d, J=7.6, 1H), 7.16 (d, J=9.6, 1H), 7.02 (t, J=7.5, 1H), 4.70 (s, 2H).

### Example 66

### N-[(1R)-1-(3-fluorophenyl)ethyl]-6-pyridin-4-yl-1,3-benzothiazol-2-amine

### Example 66A

Example 66A was synthesized following the procedure for Example 1A, substituting (R)-1-(3-fluorophenyl)ethanamine for benzyl amine.

### Example 66B

### N-[(1R)-1-(3-fluorophenyl)ethyl]-6-pyridin-4-yl-1,3-benzothiazol-2-amine

The TFA salt of the title compound (0.17 g) was synthesized following the procedure from Example 1B, substituting Example 66A for Example 1A. ¹H NMR (300 MHz, METHANOL-d₄) δ ppm 8.78-8.72 (m, 2H), 8.35 (dd, J=1.6, 7.6, 3H), 7.93 (dd, J=2.1, 8.6, 1H), 7.58 (d, J=8.6, 1H), 7.37 (td, J=5.9, 8.0, 1H), 7.25 (d, J=7.8, 1H), 7.21-7.13 (m, 1H), 7.05-6.94 (m, 1H), 5.13 (q, J=6.9, 1H), 1.61 (d, J=7.0, 3H).

### Example 67

### N-(3-fluorobenzyl)-6-(1H-pyrazol-5-yl)-1,3-benzothiazol-2-amine

Example 67 (0.029 g) was synthesized following the procedure from Example 1B, substituting Example 31A for Example 1A and substituting 1H-pyrazol-5-ylboronic acid for 4-(4,4,5,5-tetramethyl-1,3,2-dioxaborolan-2-yl)pyridine. ¹H NMR (300 MHz, METHANOL-d₄) δ ppm 8.10 (d, J=1.6, 1H), 7.80 (dd, J=1.8, 8.5, 1H), 7.69 (d, J=2.3, 1H) 7.55-7.35 (m, 2H), 7.29-7.14 (m, 2H), 7.06 (td, J=2.2, 8.3, 1H), 6.69 (d, J=2.3, 1H), 4.71 (s, 2H).

### Example 68

### N-[(1R)-1-(3-ethoxyphenyl)ethyl]-6-pyridin-4-yl-1,3-benzothiazol-2-amine

### Example 68A

Example 68A was synthesized following the procedure for Example 1A, substituting (R)-1-(3-ethoxyphenyl)ethanamine for benzyl amine.

### Example 68B

### N-[(1R)-1-(3-ethoxyphenyl)ethyl]-6-pyridin-4-yl-1,3-benzothiazol-2-amine

The TFA salt of the title compound (0.150 g) was synthesized following the procedure from Example 1B, substituting Example 68A for Example 1A. ¹H NMR (300 MHz, METHANOL-d₄) δ ppm 8.79-8.73 (m, 2H), 8.38-8.32 (m, 3H), 7.95 (dd, J=2.0, 8.6, 1H), 7.60 (d, J=8.7, 1H), 7.31-7.22 (m, 1H), 6.99 (dd, J=1.2, 7.8, 2H), 6.86-6.78 (m, 1H), 5.03 (q, J=6.8, 1H), 4.03 (q, J =7.0, 2H), 1.62 (d, J=6.8, 3H), 1.37 (t, J=7.0, 3H).

### Example 69

### 3-{[(6-pyridin-4-yl-1,3-benzothiazol-2-yl)amino]methyl}phenol

### Example 69A

Example 69A was synthesized following the procedure for Example 1A, substituting 3-(aminomethyl)phenol for benzyl amine.

### Example 69B

### 3-{[(6-pyridin-4-yl-1,3-benzothiazol-2-yl)amino]methyl}phenol

The TFA salt of the title compound (0.038 g) was synthesized following the procedure from Example 1B, substituting Example 69A for Example 1A. ¹H NMR (300 MHz, METHANOL-d₄) δ ppm 8.72 (d, J=6.8, 2H), 8.31 (t, J=4.2, 3H), 7.93 (dd, J=2.0, 8.6, 1H), 7.61 (d, J=8.5, 1H), 7.17 (t, J=7.8, 1H), 6.86 (d, J=7.8, 2H), 6.71 (d, J=7.7, 1H), 4.62 (s, 2H).

### Example 70

### N-(3-aminobenzyl)-6-pyridin-4-yl-1,3-benzothiazol-2-amine

### Example 70A

Example 70A was synthesized following the procedure for Example 1A, substituting 3-(aminomethyl)aniline for benzyl amine.

### Example 70B

### N-(3-aminobenzyl)-6-pyridin-4-yl-1,3-benzothiazol-2-amine

The TFA salt of the title compound (0.034 g) was synthesized following the procedure from Example 1B, substituting Example 70A for Example 1A. ¹H NMR (300 MHz, METHANOL-d₄) δ ppm 8.76 (d, J=6.9, 2H), 8.37 (dd, J=2.4, 4.6, 3H), 7.95 (dd, J=2.1, 8.6, 1H), 7.65-7.49 (m, 3H), 7.45 (dd, J=0.8, 1.5, 1H), 7.31 (ddd, J=2.1, 5.1, 7.8, 1H), 4.79 (s, 2H).

### Example 71

### N-[4-(aminomethyl)benzyl]-6-pyridin-4-yl-1,3-benzothiazol-2-amine

### Example 71A

Example 71A was synthesized following the procedure for Example 1A, substituting tert-butyl 4-(aminomethyl)benzylcarbamate for benzyl amine.

### Example 71B

### N-[4-(aminomethyl)benzyl]-6-pyridin-4-yl-1,3-benzothiazol-2-amine

An aqueous solution of 2M cesium carbonate(1.2 mL, 2.8 mmol) was added to a reaction flask containing the product from Example 71A (0.2 g, 0.69 mmol), 4-(4,4,5,5-tetramethyl-1,3,2-dioxaborolan-2-yl)pyridine (0.156 g, 0.76 mmol) and Pd(dppf)Cl₂.CH₂Cl₂ (0.048 g, 0.07 mmol) in dioxane (3.45 mL, 0.2 M). The reaction mixture was heated at 95° C. under N₂ for 12 hours. The cooled reaction mixture was diluted with dichloromethane (10 mL) and washed with saturated NaHCO₃ (2×20 mL) and water (2×10 mL), and concentrated. The residue was treated with 1 mL dichloromethane/trifluoroacetic acid (1:1) for 2 hours at 25° C. The reaction mixture was then concentrated and purified via HPLC yielding 0.144 g of the title compound as a TFA salt. ¹H NMR (300 MHz, METHANOL-d₄) δ ppm 8.53 (dd, J=1.6, 4.6, 2H), 8.06 (d, J=1.9, 1H), 7.75-7.66 (m, 3H), 7.56-7.33 (m, 5H), 4.71 (s, 2H), 4.09 (s, 2H).

### Example 72

### N-[3-(2-morpholin-4-ylethoxy)benzyl]-6-pyridin-4-yl-1,3-benzothiazol-2-amine

### Example 72A

Example 72A was synthesized following the procedure for Example 1A, substituting (3-(2-morpholinoethoxy)phenyl)methanamine for benzyl amine.

### Example 72B

### N-[3-(2-morpholin-4-ylethoxy)benzyl]-6-pyridin-4-yl-1,3-benzothiazol-2-amine

The TFA salt of the title compound (0.063 g) was synthesized following the procedure from Example 1B, substituting Example 72A for Example 1A. ¹H NMR (300 MHz, CDCl₃) δ ppm 8.64 (dd, J=1.6, 4.5, 2H), 7.88 (d, J=1.1, 1H), 7.71-7.58 (m, 2H), 7.51 (dd J=1.7, 4.5, 2H), 7.30 (d, J=7.9, 1H), 7.06-6.79 (m, 3H), 5.69 (s, 1H), 4.65 (s, 2H), 4.11 (t, J=5.7, 2H), 3.80-3.65 (m, 4H), 2.80 (t, J=5.7, 2H), 2.64-2.50 (m, 4H), 2.11 (s, 0H).

### Example 73

### N-(3-fluorobenzyl)-6-(2-methylpyridin-4-yl)-1,3-benzothiazol-2-amine

The TFA salt of the title compound (0.154 g) was synthesized following the procedure from Example 1B, substituting 2-methylpyridin-4-ylboronic acid for 4-(4,4,5,5-tetramethyl-1,3,2-dioxaborolan-2-yl)pyridine and substituting Example 31A for Example 1A.

¹H NMR (300 MHz, METHANOL-d₄) δ ppm 8.59 (d, J=6.5, 1H), 8.32 (d, J=1.9, 1H), 8.23 (d, J=1.1, 1H), 8.16 (dd, J=1.8, 6.5, 1H), 7.92 (dd, J=2.0, 8.6, 1H), 7.61 (d, J=8.5, 1H), 7.43-7.32 (m, 1H), 7.23 (d, J=7.7, 1H), 7.15 (d, J=9.9, 1H), 7.02 (t, J=8.5, 1H), 4.74 (d, J=17.1, 2H), 2.80 (s, 3H).

### Example 74

### 3-({[6-(1H-pyrrolo[2,3-b]pyridin-4-yl)-1,3-benzothiazol-2-yl]amino}methyl)phenol

Example 74 (0.020 g) was synthesized following the procedure from Example 6B, substituting Example 69A for Example 2A. ¹H NMR (500 MHz, METHANOL-d₄) δ ppm 8.39 (d, J=6.1, 1H), 8.24 (d, J=1.8, 1H), 7.88 (dd, J=1.9, 8.4, 1H), 7.73-7.61 (m, 3H), 7.19 (t, J=7.8, 1H), 7.03 (d, J=3.6, 1H), 6.89-6.85 (m, 1H), 6.92-6.86 (m, 1H), 6.74 (dd, J=2.0, 8.0, 1H), 4.65 (s, 2H).

### Example 75

### N-{3-[2-(dimethylamino)ethoxy]benzyl}-6-pyridin-4-yl-1,3-benzothiazol-2-amine

### Example 75A

Example 75A was synthesized following the procedure for Example 1A, substituting 2-(3-(aminomethyl)phenoxy)-N,N-dimethylethanamine for benzyl amine.

### Example 75B

### N-{3-[2-(dimethylamino)ethoxy]benzyl}-6-pyridin-4-yl-1,3-benzothiazol-2-amine

The TFA salt of the title compound (0.019 g) was synthesized following the procedure from Example 1B, substituting Example 75A for Example 1A. ¹H NMR (500 MHz, METHANOL-d₄) δ ppm 8.54 (d, J=6.1, 2H), 8.06 (t, J=2.5, 1H), 7.54 (d, J=8.4, 1H), 7.29 (t, J=7.9, 1H), 7.03 (d, J=8.3, 2H), 6.93-6.83 (m, 1H), 4.65 (s, 2H), 4.20 (t, J=5.3, 2H), 3.10 (t, J=5.2, 2H), 2.59 (d, J=5.4, 6H).

### Example 76

### 6-(3-fluoropyridin-4-yl)-N-[3-(2-morpholin-4-ylethoxy)benzyl]-1,3-benzothiazol-2-amine

The TFA salt of the title compound (0.027 g) was synthesized following the procedure from Example 1B, substituting 2-fluoropyridin-4-ylboronic acid for 4-(4,4,5,5-tetramethyl-1,3,2-dioxaborolan-2-yl)pyridine and substituting Example 72A for Example 1A.

¹H NMR (300 MHz, METHANOL-d₄) δ ppm 8.65 (d, J=3.4, 1H), 8.50 (d, J=5.3, 1H), 8.08 (s, 1H), 7.84-7.77 (m, 1H), 7.76-7.68 (m, 1H), 7.59 (d, J=8.5, 1H), 7.35 (t, J=8.1, 1H), 7.09 (d, J=6.8, 2H), 6.98 (d, J=8.1, 1H), 4.70 (s, 2H), 4.49-4.34 (m, 2H), 4.03 (s, 2H) 3.78 (s, 2H), 3.61 (dd, J=12.6, 17.4, 4H), 3.29-3.15 (m, 2H).

### Example 77

### 6-[3-(aminomethyl)pyridin-4-yl]-N-(3-fluorobenzyl)-1,3-benzothiazol-2-amine

The TFA salt of the title compound (0.037 g) was synthesized following the procedure from Example 71B, substituting Example 31A for Example 71A and substituting 3-((tert-butoxycarbonylamino)methyl)pyridin-4-ylboronic acid for 4-(4,4,5,5-tetramethyl-1,3,2-dioxaborolan-2-yl)pyridine. ¹H NMR (300 MHz, METHANOL-d₄) δ ppm 8.82 (s, 1H), 8.71 (d, J=5.4, 1H), 7.78 (d, J=1.7, 1H), 7.66 (d, J=5.5, 1H), 7.62 (d, J=8.3, 1H), 7.44-7.33 (m, 2H), 7.23 (d, J=7.6, 1H), 7.16 (d, J=9.8, 1H), 7.03 (td, J=2.4, 8.6, 1H), 4.72 (s, 2H), 4.34 (s, 2H).

### Example 78

### N-[3-(2-morpholin-4-ylethoxy)benzyl]-6-(1H-pyrrolo[2,3-b]pyridin-4-yl)-1,3-benzothiazol-2-amine

Example 78 (0.053 g) was synthesized following the procedure from Example 6B, substituting Example72A for Example 2A. ¹H NMR (500 MHz. METHANOL-d₄) δ ppm 3.19-3.31 (m, 2H) 3.49-3.71 (m, 4H) 3.81 (s, 2H) 4.04 (s, 2H) 4.34-4.46 (m, 2H) 4.72 (s, 2H) 7.05 (d, J=3.66 Hz, 1H) 7.07-7.13 (m, 3H) 7.28-7.41 (m, 1H) 7.64-7.69 (m, 2 H) 7.72 (d, J=3.66 Hz, 1H) 7.88 (dd, J=8.54, 1.83 Hz, 1H) 8.24 (d, J=1.83 Hz, 1H) 8.41 (d, J=6.41 Hz, 1 H).

### Example 79

### N-{3-[3-(dimethylamino)propoxyl]benzyl}-6-pyridin-4-yl-1,3-benzothiazol-2-amine

### Example 79A

Example 79A was synthesized following the procedure for Example 1A, substituting 3-(3-(aminomethyl)phenoxy)-N,N-dimethylpropan-1-amine for benzyl amine.

### Example 79B

### N-{3-[3-(dimethylamino)propoxy]benzyl}-6-pyridin-4-yl-1,3-benzothiazol-2-amine

The TFA salt of the title compound (0.003 g) was synthesized following the procedure from Example 1B, substituting Example 79A for Example 1A. ¹H NMR (500 MHz, METHANOL-d₄) δ ppm 8.54 (d, J=6.3, 2H), 8.06 (d, J=1.8, 1H), 7.71 (ddd, J=1.8, 6.6, 10.4, 3H), 7.54 (d, J=8.4, 1H), 7.27 (t, J=8.2, 1H), 7.00 (d, J=7.7, 2H), 6.91-6.80 (m, 1H), 4.64 (s, 2H), 4.17-3.97 (m, 2H), 3.08-2.95 (m, 2H), 2.65 (s, 6H).

### Example 80

### N-(2,3-dihydro-1,4-benzodioxin-5-ylmethyl)-6-(3-fluoropyridin-4-yl)-1,3-benzothiazol-2-amine

The TFA salt of the title compound (0.131 g) was synthesized following the procedure from Example 1B, substituting Example 12A for Example 1A and substituting 3-fluoropyridin-4-ylboronic acid for 4-(4,4,5,5-tetramethyl-1,3,2-dioxaborolan-2-yl)pyridine.

¹H NMR (300 MHz, METHANOL-d₄) δ ppm 8.59 (d, J=3.3, 1H), 8.47 (d, J=5.2, 1H), 8.08 (s, 1H), 7.72 (t, J=6.2, 2H), 7.61 (d, J=8.5, 1H) 6.92 (d, J=3.3, 1H), 6.90-6.76 (m, 2H), 4.67 (s, 2H), 4.36-4.30 (m, 2H), 4.29-4.24 (m, 2H).

### Example 81

### (2S)-2-{[6-(3-fluoropyridin-4-yl)-1,3-benzothiazol-2-yl]amino}-2-phenylethanol

The TFA salt of the title compound (0.005 g) was synthesized following the procedure from Example 1B, substituting 3-fluoropyridin-4-ylboronic acid for 4-(4,4,5,5-tetramethyl-1,3,2-dioxaborolan-2-yl)pyridine and substituting Example 51A for Example 1A.

¹H NMR (500 MHz, METHANOL-d₄) δ ppm 8.59 (s, 1H), 8.47 (d, J=5.4, 1H), 8.05 (s, 1H), 7.76-7.66 (m, 2H), 7.56 (d, J=8.5, 1H), 7.48-7.36 (m, 4H), 7.31 (t, J=7.3, 1H), 5.04 (s, 1H), 3.91 (dt, J=8.9, 17.7, 1H), 3.88 (s, 1H).

### Example 82

### 3-{[(6-pyridin-4-yl-1,3-benzothiazol-2-yl)(2-pyrrolidin-1-ylethyl)amino]methyl}phenol

Example 69B (0.1 g, 0.183 mmol) was dissolved in 1 mL N,N-dimethyl formamide and treated with 60% NaH (0.011 g, 0.19 mmol) and stirred at 25° C. for 1 hour. To this was added 1-(2-chloroethyl)pyrrolidine (0.025 g, 0.19 mmol) and the reaction mixture was then heated at 100° C. under a steady stream of N₂ for 12 hours. The cooled reaction mixture was then poured into water (5 mL) and washed 3× with dichloromethane (3×5 mL). The combined organic fractions were concentrated and purified via HPLC to afford 0.015 g of the title compound as a TFA salt. ¹H NMR (500 MHz, METHANOL-d₄) δ ppm 8.54 (d, J=6.2, 2H), 8.14 (d, J=1.8, 1H), 7.78-7.67 (m, 3H), 7.61 (d, J=8.4, 1H), 7.18 (dd, J=6.1, 13.9, 1H), 6.82 (dd, J=11.2. 18.8, 2H), 6.74 (d, J=7.6, 1H), 4.78 (s, 2H), 3.84 (t, J=7.1, 2H), 3.12-2.83 (m, 6H), 1.92 (m, J=8.6, 4H).

### Example 83

### N-[3-(morpholin-4-ylmethyl)benzyl]-6-pyridin-4-yl-1,3-benzothiazol-2-amine

### Example 83A

Example 83A was synthesized following the procedure for Example 1A, substituting (3-(morpholinomethyl)phenyl)methanamine for benzyl amine.

### Example 83B

### N-[3-(morpholin-4-ylmethyl)benzyl]-6-pyridin-4-yl-1,3-benzothiazol-2-amine

The TFA salt of the title compound (0.051 g) was synthesized following the procedure from Example 1B, substituting Example 83A for Example 1A. ¹H NMR (300 MHz, METHANOL-d₄) δ ppm 8.53 (dd, J=1.6, 4.7, 2H), 8.04 (dd, J=1.9, 12.9, 1H), 7.78-7.62 (m, 3H), 7.53 (d, J=8.5, 1H), 7.41 (s, 1H), 7.37-7.19 (m, 3H), 4.67 (s, 2H), 3.69-3.58 (m, 4H), 3.52 (s, 2H), 3.07 (s, 0H), 2.50-2.36 (m, 4H), 1.99 (d, J=25.3, 0H).

### Example 84

### N-[3-(2-piperidin-1-ylethoxy)benzyl]-6-pyridin-4-yl-1,3-benzothiazol-2-amine

### Example 84A

Example 84A was synthesized following the procedure for Example 1A, substituting (3-(2-(piperidin-1-yl)ethoxy)phenyl)methanamine for benzyl amine.

### Example 84B

### N-[3-(2-piperidin-1-ylethoxy)benzyl]-6-pyridin-4-yl-1,3-benzothiazol-2-amine

The TFA salt of the title compound (0.009 g) was synthesized following the procedure from Example 1B, substituting Example 84A for Example 1A. ¹H NMR (500 MHz, METHANOL-d₄) δ ppm 8.75 (d, J=6.9, 2H), 8.44-8.31 (m, 3H), 8.02-7.91 (m, 2H), 7.63 (dd, J=9.9. 14.6, 2H), 7.34 (t, J=8.1, 1H), 7.08 (d, J=8.0, 1H), 4.45-4.30 (m, 2H), 3.70-3.39 (m, 4H), 3.06 (t, J=12.5, 2H), 1.88 (dd, J=20.9, 85.0, 6H), 1.54 (s, 1H).

### Example 85

### N-(3-{[(6-pyridin-4-yl-1,3-benzothiazol-2-yl)amino]methyl}phenyl)methanesulfonamide

### Example 85A

Example 85A was synthesized following the procedure for Example 1A, substituting N-(3-(aminomethyl)phenyl)methanesulfonamide for benzyl amine.

### Example 85B

### N-(3-{[(6-pyridin-4-yl-1,3-benzothiazol-2-yl)amino]methyl}phenyl)methanesulfonamide

The TFA salt of the title compound (0.071 g) was synthesized following the procedure from Example 1B, substituting Example 85A for Example 1A. ¹H NMR (300 MHz, METHANOL-d₄) δ ppm 8.75 (d, J=6.8, 2H), 8.40-8.33 (m, 3H), 7.95 (dd, J=2.0, 8.7, 1H), 7.62 (d, J=8.6, 1H), 7.34 (dd, J=6.5, 9.1, 2H), 7.18 (t, J=8.0, 2H), 4.70 (s, 2H), 2.93 (s, 3H).

### Example 86

### N-(2,3-dihydro-1,4-benzodioxin-5-ylmethyl)-6-pyrimidin-4-yl-1,3-benzothiazol-2-amine

An aqueous solution of 2 M cesium carbonate(1.2 mL, 2.8 mmol) was added to a reaction flask containing the product from Example 12A (0.2 g, 0.69 mmol), 4,4,4′,4′,5,5,5′,5′-octamethyl-2,2′-bi(1,3,2-dioxaborolane) (0.156 g, 0.76 mmol) and Pd(dppf)Cl₂.CH₂Cl₂ (0.048 g, 0.07 mmol) in dioxane (3.45 mL, 0.2 M). The reaction mixture was heated at 95° C. under N₂ for 2 hours. To the cooled vessel was then added 4-chloropyrimidine (0.114 g, 1.04 mmol) and the reaction mixture was heated in a microwave for 60 min at 150° C. The cooled reaction mixture was diluted with dichloromethane (10 mL) and washed with saturated NaHCO₃ (2×20 mL) and water (2×10 mL). The organic layer was concentrated and the residue was purified via HPLC to afford 0.015 g of the title compound as a TFA salt. ¹H NMR (300 MHz, METHANOL-d₄) δ ppm 9.12 (s, 1H), 8.71 (d, J=5.6, 1H), 8.49 (d, J=1.8, 1H), 8.11 (dd, J=1.8, 8.5, 1H), 7.97 (d, J=5.6, 1H), 7.53 (d, J=8.6, 2H), 6.78 (d, J=4.5, 2H), 4.63 (s, 2H), 4.33 (dd, J=2.3, 5.6, 2H), 4.26 (dd, J=2.2, 5.5. 2H).

### Example 87

### N-[3-(4-methylpiperazin-1-yl)benzyl]-6-pyridin-4-yl-1,3-benzothiazol-2-amine

### Example 87A

Example 87A was synthesized following the procedure for Example 1A, substituting (3-(4-methylpiperazin-1-yl)phenyl)methanamine for benzyl amine.

### Example 87B

### N-[3-(4-methylpiperazin-1-yl)benzyl]-6-pyridin-4-yl-1,3-benzothiazol-2-amine

The TFA salt of the title compound (0.012 g) was synthesized following the procedure from Example 1B, substituting Example 87A for Example 1A. ¹H NMR (300 MHz, METHANOL-d₄) δ ppm 8.76 (d, J=7.1, 2H), 8.38-8.33 (m, 3H), 7.98-7.93 (m, 1H), 7.63 (dd, J=5.8, 7.7, 1H), 7.30 (t, J=7.9, 1H), 7.09 (s, 1H), 6.99 (dd, J=5.3, 13.7, 2H), 4.68 (s, 2H), 3.86 (d, J=13.0, 2 H), 3.58 (s, 2H), 3.23 (s, 2H), 3.05 (s, 2H), 2.96 (s, 3H).

### Example 88

### N-[3-(2-morpholin-4-ylethoxy)benzyl]-6-pyrimidin-4-yl-1,3-benzothiazol-2-amine

The TFA salt of the title compound (0.002 g) was synthesized following the procedure from Example 86, substituting Example 72A for Example 12A. ¹H NMR (300 MHz, METHANOL-d₄) δ ppm 9.12 (d, J=1.2, 1H), 8.71 (d. J=5.6, 1H), 8.50 (d, J=1.9, 1H), 8.11 (dd, J=1.9, 8.5, 1H), 7.97 (dd, J=1.4, 5.5, 1H), 7.54 (d, J=8.5, 1H), 7.28 (t, J=8.1, 1H), 7.01 (d, J=6.8, 2H), 6.92-6.84 (m, 1H), 4.66 (s, 2H), 4.18 (t, J=5.4, 2H), 3.77-3.69 (m, 4H), 2.95 (t, J=5.4, 2H), 2.77-2.70 (m, 4H).

### Example 89

### N-[3-(2-piperidin-1-ylethoxy)benzyl]-6-pyrimidin-4-yl-1,3-benzothiazol-2-amine

The TFA salt of the title compound (0.007 g) was synthesized following the procedure from Example 86, substituting Example 84A for Example 12A. ¹H NMR (300 MHz, METHANOL-d₄) δ ppm 9.15 (s, 1H), 8.74 (d, J=5.6, 1H), 8.54 (d, J=1.7, 1H), 8.16 (dd, J=1.8, 8.6, 1H), 8.01 (d, J=5.6, 1H), 7.56 (d, J=7.1, 1H), 7.34 (t, J=8.1, 2H), 7.09 (s, 3H), 4.67 (d, J=13.3, 3H), 4.43-4.30 (m, 3H), 3.56 (dd, J=11.5, 16.5, 7H), 3.03 (d, J=12.0, 4H), 1.86 (d, J=33.4, 11H).

### Example 90

### N-(3-fluorobenzyl)-6-pyrimidin-4-yl-1,3-benzothiazol-2-amine

The TFA salt of the title compound (0.003 g) was synthesized following the procedure from Example 86, substituting Example 31A for Example 12A. ¹H NMR (300 MHz, METHANOL-d₄) δ ppm 9.15 (s, 1H), 8.74 (d, J=5.6, 1H), 8.55 (d, J=1.6, 1H), 8.17 (dd, J=1.8, 8.6, 1H), 8.01 (dd, J=1.2, 5.6, 1H), 7.62-7.52 (m, 2H), 7.20 (dd, J=8.8, 21.9, 2H), 7.04 (dd, J=5.4, 11.6, 1H), 4.71 (s, 2H).

### Example 91

### 6-pyrimidin-4-yl-N-[3-(2-pyrrolidin-1-ylethoxy)benzyl]-1,3-benzothiazol-2-amine

### Example 91A

Example 91A was synthesized following the procedure for Example 1A, substituting (3-(2-(pyrrolidin-1-yl)ethoxy)phenyl)methanamine for benzyl amine.

### Example 91B

The TFA salt of the title compound (0.001 g) was synthesized following the procedure from Example 86, substituting Example 91A for Example 12A.

### Example 92

### N-[3-(2-morpholin-4-ylethoxy)benzyl]-6-(7H-pyrrolo[2,3-d]pyrimidin-4-yl-1,3-benzothiazol-2-amine

Example 92 (0.01 g) was synthesized following the procedure from Example 71B, substituting tert-butyl 4-(4,4,5,5-tetramethyl-1,3-dioxolan-2-yl)-7H-pyrrolo[2,3-d]pyrimidine-7-carboxylate for 4-(4,4,5,5-tetramethyl-1,3,2-dioxaborolan-2-yl)pyridine, and substituting Example 72A for Example 71A. ¹H NMR (300 MHz, METHANOL-d₄) δ ppm 8.75 (s, 1H), 8.37 (d, J=1.6, 1H), 8.05 (dd, J=1.8, 8.5, 1H), 7.62 (s, 1H), 7.62-7.50 (m, 2H), 7.28 (t, J=7.9, 1H), 7.02 (s, 1H), 7.00 (s, 1H), 7.00-6.81 (m, 2H), 4.67 (s, 2H), 4.16 (t, J=5.5, 2H), 3.73-3.66 (m, 4H), 2.84 (t, J=5.5, 2H), 2.63 (dd, J=3.7, 8.4, 4H), 1.98 (s, 1H).

### Example 93

### 6-(2-fluoropyridin-4-yl)-N-[3-(2-morpholin-4-ylethoxy)benzyl]-1,3-benzothiazol-2-amine

The TFA salt of the title compound (0.14 g) was synthesized following the procedure from Example 1B, substituting 2-fluoropyridin-4-ylboronic acid for 4-(4,4,5,5-tetramethyl-1,3,2-dioxaborolan-2-yl)pyridine and substituting Example 72A for Example 1A. ¹H NMR (500 MHz, METHANOL-d₄) δ ppm 8.22 (d, J=5.4, 1H), 8.12 (d, J=1.9, 1H), 7.76 (dd, J=1.9, 8.5, 1H), 7.62 (d, J=5.4, 1H), 7.56 (d, J=8.5, 1H), 7.40-7.34 (m, 2H), 7.09 (d, J=7.5, 2H), 6.97 (d, J=7.1, 1H), 4.69 (s, 2H), 4.42-4.36 (m, 2H), 4.04 (s, 2H), 3.78 (s, 2H), 3.61 (dd, J=19.1, 24.0, 4H), 3.29-3.18 (m, 2H).

### Example 94

### 6-(2-fluoropyridin-4-yl)-N-(2-piperidin-1-ylethyl)-1,3-benzothiazol-2-amine

### Example 94A

Example 94A was synthesized following the procedure for Example 1A, substituting 2-(piperidin-1-yl)ethanamine for benzyl amine.

### Example 94B

### 6-(2-fluoropyridin-4-yl)-N-(2-piperidin-1-ylethyl)-1,3-benzothiazol-2-amine

The TFA salt of the title compound (0.118 g) was synthesized following the procedure from Example 1B, substituting Example 94A for Example 1A and substituting 2-fluoropyridin-4-ylboronic acid for 4-(4,4,5,5-tetramethyl-1,3,2-dioxaborolan-2-yl)pyridine.

¹H NMR (300 MHz, METHANOL-d₄) δ ppm 8.22 (d, J=5.4, 1H), 8.14 (d, J=1.9, 1H), 7.76 (dd, J=2.0, 8.5, 1H), 7.61 (dd, J=3.9, 6.9, 2H), 7.38 (s, 1H), 3.94-3.86 (m, 2H), 3.72 (s, 2H), 3.46-3.39 (m, 2H), 3.07 (s, 2H), 1.88 (s, 5H), 1.68-1.52 (m, 1H).

### Example 95

### 6-(2-fluoropyridin-4-yl)-N-(3-methoxybenzyl)-N-(2-piperidin-1-ylethyl)-1,3-benzothiazol-2-amine

### Example 95A

Example 94A (0.2 g, 0.22 mmol) was dissolved in N,N-dimethyl formamide (1 mL) and treated with 60% NaH (0.015 g, 0.22 mmol) and stirred at 25° C. for 1 hour. To the reaction mixture was added 1-(bromomethyl)-3-methoxybenzene and the reaction mixture was heated at 75° C. for an additional 12 hours. The cooled reaction mixture was diluted with dichloromethane (5 mL) and washed with saturated NaHCO₃ (2×10 mL) and water (2×10 mL). The organic layer was separated and concentrated to a yellow oil and used in the next step without further purification.

### Example 95B

### 6-(2-fluoropyridin-4-yl)-N-(3-methoxybenzyl)-N-(2-piperidin-1-ylethyl)-1,3-benzothiazol-2-amine

The TFA salt of the title compound (0.193 g) was synthesized following the procedure from Example 1B, substituting Example 95A for Example 1A and substituting 2-fluoropyridin-4-ylboronic acid for 4-(4,4,5,5-tetramethyl-1,3,2-dioxaborolan-2-yl)pyridine.

¹H NMR (300 MHz, METHANOL-d₄) δ ppm 8.22 (d, J=5.4, 1H), 8.14 (d, J=1.9, 1H), 7.76 (dd J=2.0, 8.5, 1H), 7.61 (dd, J=3.9, 6.9, 2H), 7.38 (s, 1H), 3.94-3.86 (m, 2H), 3.72 (s, 2H), 3.46-3.39 (m, 2H), 3.07 (s, 2H), 1.88 (s, 5H), 1.68-1.52 (m, 1H).

### Example 96

### methyl 3-{[(6-pyridin-4-yl-1,3-benzothiazol-2-yl)amino]-methyl}benzoate

### Example 96A

Example 96A was synthesized following the procedure for Example 1A substituting methyl 3-(aminomethyl)benzoate for benzyl amine.

### Example 96B

### methyl 3-{[(6-pyridin-4-yl-1,3-benzothiazol-2-yl)amino]methyl}benzoate

The TFA salt of the title compound (0.052 g) was synthesized following the procedure from Example 1B, substituting Example 96A for Example 1A. ¹H NMR (300 MHz, METHANOL-d₄) δ ppm 8.78-8.72 (m, 2H), 8.36 (dd, J=1.7, 5.3, 3H), 8.09 (s, 1H), 7.99-7.91 (m, 2H), 7.71-7.63 (m, 1H), 7.64 (s, 1H), 7.49 (t, J=7.8, 1H), 4.77 (s, 2H), 3.90 (s, 3H).

### Example 97

### N-(3-methoxybenzyl)-N′,N′-dimethyl-N-(6-pyridin-4-yl-1,3-benzothiazol-2-yl)ethane-1,2-diamine

### Example 97A

Example 97A was synthesized following the procedure for Example 1A substituting N¹,N¹-dimethylethane-1,2-diamine for benzyl amine.

### Example 97B

Example 97B (0.22 g) was synthesized following the procedure from Example 95A, substituting Example 97A for Example 87A. ¹H NMR (300 MHz, METHANOL-d₄) δ ppm 8.22 (d, J=5.4, 1H), 8.14 (d, J=1.9, 1H), 7.76 (dd, J=2.0, 8.5, 1H), 7.61 (dd, J=3.9, 6.9, 2H), 7.38 (s, 1H), 3.94-3.86 (m, 2H), 3.72 (s, 2H), 3.46-3.39 (m, 2H), 3.07 (s, 2H), 1.88 (s, 5H), 1.68-1.52 (m, 1H).

### Example 97C

### N-(3-methoxybenzyl)-N′,N′-dimethyl-N-(6-pyridin-4-yl-1,3-benzothiazol-2-yl)ethane-1,2-diamine

The TFA salt of the title compound (0.041 g) was synthesized following the procedure from Example 1B, substituting Example 97B for Example 1A. ¹H NMR (300 MHz, METHANOL-d₄) δ ppm 8.77 (d, J=7.0, 2H), 8.43 (d, J=1.9, 1H), 8.39-8.33 (m, 2H). 8.00 (dd, J=2.0, 8.6, 1H), 7.76 (d, J=8.6, 1H), 7.36-7.26 (m, 1H), 6.97-6.88 (m, 3H), 4.80 (s, 2H), 4.13 (t, J=6.3, 2H), 3.79 (d, J=4.2, 3H), 3.49 (t, J=6.3, 2H), 3.03 (s, 6H).

### Example 98

### N-[2-(dimethylamino)ethyl]-N-methyl-3-{[(6-pyridin-4-yl-1,3-benzothiazol-2-yl)amino]methyl}benzamide

### Example 98A

Example 96A (1 g, 2.67 mmol) was dissolved in dioxane (10 mL) and treated with aqueous 1N NaOH (3 mL, 3 mmol) and heated at 80° C. for 3 hours. The cooled reaction mixture was diluted with dichloromethane (50 mL) and washed with saturated NaHCO₃ (4×20 mL) and water (2×10 mL). The organic fractions were concentrated to the title compound (0.91 g) as a white solid.

### Example 98B

Example 98A (0.2 g, 0.551 mmol) was dissolved in N,N-dimethyl formamide (1 mL) and treated with N-ethyl-N-isopropylpropan-2-amine (0.071 g, 0.551 mmol), and 2-(3H-[1.2.3]triazolo[4.5-b]pyridin-3-yl)-1,1,3,3-tetramethylisouronium hexafluorophosphate(V) (0.209 g, 0.551 mmol) and stirred at 25° C. for 10 minutes. To this was added N¹,N¹,N²-trimethylethane-1,2-diamine (0.065 g, 0.551 mmol) and the reaction mixture was stirred for an additional 8 hours at 25° C. To the reaction mixture was added water (2 mL) and the tan precipitate was filtered, and washed with water(2×10 mL) and hexanes(2×10 mL) to provide 0.25 g of the title compound that was used directly in the next step.

### Example 98C

### N-[2-(dimethylamino)ethyl]-N-methyl-3-{[(6-pyridin-4-yl-1,3-benzothiazol-2-yl)amino]methyl}benzamide

The TFA salt of the title compound (0.082 g) was synthesized following the procedure from Example 1B, substituting Example 98B for Example 1A. ¹H NMR (300 MHz, METHANOL-d₄) δ ppm 8.81-8.70 (m, 2H), 8.36 (dd, J=2.5, 4.6, 3H), 7.95 (dd, J=2.1, 8.5, 1H), 7.52 (ddd, J=8.1, 16.2, 21.2, 5H), 4.77 (d, J=3.1, 2H), 3.90 (s, 2H), 3.46 (d, J=5.8, 2H), 3.03 (d, J=11.8, 9H).

### Example 99

### N-[2-(dimethylamino)ethyl]-3-{[(6-pyridin-4-yl-1,3-benzothiazol-2-yl)amino]methyl}benzamide

### Example 99A

Example 99A (0.15 g) was synthesized following the procedure from Example 98B, substituting N¹,N¹-dimethylethane-1,2-diamine for N¹,N¹,N²-trimethylethane-1,2-diamine.

### Example 99B

### N-[2-(dimethylamino)ethyl]-3-{[(6-pyridin-4-yl-1,3-benzothiazol-2-yl)amino]methyl}benzamide

The TFA salt of the title compound (0.080 g) was synthesized following the procedure from Example 1B, substituting Example 99A for Example 1A. ¹H NMR (300 MHz, METHANOL-d₄) δ ppm 8.79-8.73 (m, 2H), 8.37 (dd, J=2.6, 4.5, 3H), 7.96 (dd, J=2.1, 8.5, 2H), 7.84-7.77 (m, 1H), 7.64 (dd, J=4.6, 11.9, 2H), 7.50 (t, J=7.8, 1H), 4.79 (s, 2H), 4.78-4.71 (m, 0H), 3.76 (t, J=5.9, 2H), 3.36 (dd, J=6.8, 12.5, 2H), 2.98 (s, 6H).

### Example 100

### N-(2-hydroxyethyl)-3-{[(6-pyridin-4-yl-1,3-benzothiazol-2-yl)amino]methyl}benzamide

### Example 100A

Example 100A (0.18 g) was synthesized following the procedure from Example 98B, substituting 2-aminoethanol for N¹,N¹,N²-trimethylethane-1,2-diamine.

### Example 100B

### N-(2-hydroxyethyl)-3-{[(6-pyridin-4-yl-1,3-benzothiazol-2-yl)amino]methyl}benzamide

The TFA salt of the title compound (0.080 g) was synthesized following the procedure from Example 1B, substituting Example 100A for Example 1A. ¹H NMR (300 MHz, METHANOL-d₄) δ ppm 8.78-8.71 (m, 2H), 8.39-8.31 (m, 3H), 7.94 (dt, J=7.0, 8.0, 2H), 7.76 (d, J=8.0, 1H), 7.67-7.56 (m, 2H), 7.47 (t, J=7.7, 1H), 4.77 (d, J=4.2, 2H), 3.70 (t, J=5.8, 2H), 3.55-3.45 (m, 2H).

### Example 101

### N-(2-morpholin-4-ylethyl)-3-{[(6-pyridin-4-yl-1,3-benzothiazol-2-yl)amino]methyl}benzamide

### Example 101A

Example 101A (0.25 g) was synthesized following the procedure from Example 98B, substituting 2-morpholinoethanamine for N¹,N¹,N²-trimethylethane-1,2-diamine.

### Example 101B

### N-(2-morpholin-4-ylethyl)-3-{[(6-pyridin-4-yl-1,3-benzothiazol-2-yl)amino]methyl}benzamide

The TFA salt of the title compound (0.090 g) was synthesized following the procedure from Example 1B, substituting Example 101A for Example 1A. ¹H NMR (300 MHz, METHANOL-d₄) δ ppm 8.76 (d, J=7.0, 2H), 8.36 (dd, J=2.5, 4.5, 3H), 8.02-7.92 (m, 2H), 7.80 (d, J=7.8, 1H), 7.63 (t, J=8.2, 2H), 7.50 (t, J=7.7, 1H), 4.79 (s, 2H), 4.05 (s, 2H), 3.78 (t, J=5.9, 6H), 3.41 (t, J=5.9, 2H), 3.29-3.11 (m, 2H).

### Example 102

### N-(2-hydroxyethyl)-N-methyl-3-{[(6-pyridin-4-yl-1,3-benzothiazol-2-yl)amino]methyl}benzamide

### Example 102A

Example 102A (0.24 g) was synthesized following the procedure from Example 98B, substituting 2-(methylamino)ethanol for N¹,N¹,N²-trimethylethane-1,2-diamine.

### Example 102B

### N-(2-hydroxyethyl)-N-methyl-3-{[(6-pyridin-4-yl-1,3-benzothiazol-2-yl)amino]methyl}benzamide

The TFA salt of the title compound (0.090 g) was synthesized following the procedure from Example 1B, substituting Example 102A for Example 1A. ¹H NMR (300 MHz, METHANOL-d₄) δ ppm 8.76 (d, J=6.9, 2H), 8.38-8.32 (m, 3H), 7.95 (dd, J=1.9, 8.6, 1H), 7.66-7.35 (m, 5H), 4.76 (s, 2H), 3.81 (t, J=5.5, 1H), 3.63 (dt, J=5.5, 15.7, 2H), 3.43 (dt, J=5.3, 17.3, 1H), 3.07 (d, J=23.0, 3H).

### Example 103

### N-[(1R)-1-(3-propoxyphenyl)ethyl]-6-pyridin-4-yl-1,3-benzothiazol-2-amine

### Example 103A

Example 103A was synthesized following the procedure for Example 1A, substituting (R)-1-(3-propoxyphenyl)ethanamine for benzyl amine.

### Example 103B

### N-[(1R)-1-(3-propoxyphenyl)ethyl]-6-pyridin-4-yl-1,3-benzothiazol-2-amine

The TFA salt of the title compound (0.0036 g) was synthesized following the procedure from Example 1B, substituting Example 103A for Example 1A. ¹H NMR (500 MHz, METHANOL-d₄) δ ppm 8.73 (d, J=6.8, 2H), 8.32 (dd, J=4.5, 7.6, 2H), 8.33-8.29 (m, 1H), 7.92 (dd, J=2.0, 8.6, 1H), 7.58 (d, J=8.6, 1H), 7.25 (t, J=8.0, 1H), 6.98 (t, J=4.1, 1H), 6.98 (t, J=4.1, 1H), 6.84-6.78 (m, 1H), 5.05 (d, J=6.6, 1H), 3.97-3.88 (m, 2H), 1.83-1.72 (m, 2H), 1.60 (d, J=6.9, 3H), 1.03 (t, J=7.4, 3H).

### Example 104

### 2-(2-phenylpyrrolidin-1-yl)-6-pyridin-4-yl-1,3-benzothiazole

### Example 104A

Example 104A was synthesized following the procedure for Example 1A, substituting 2-phenylpyrrolidine for benzyl amine.

### Example 104B

### 2-(2-phenylpyrrolidin-1-yl)-6-pyridin-4-yl-1,3-benzothiazole

The TFA salt of the title compound (0.008 g) was synthesized following the procedure from Example 1B, substituting Example 104A for Example 1A. ¹H NMR (300 MHz, METHANOL-d₄) δ ppm 8.84-8.66 (m, 2H), 8.46-8.24 (m, 3H), 7.95 (dd, J=2.1, 8.7, 1H), 7.64 (d, J=8.6, 1H), 7.44-7.20 (m, 5H), 5.06 (s, 1H), 4.16-3.85 (m, 2H), 2.62 (dq, J=7.9, 12.2, 1H), 2.32-1.96 (m, 3H).

### Example 105

### 6-pyridin-4-yl-2-(2-thien-2-ylpyrrolidin-1-yl)-1,3-benzothiazole

### Example 105A

Example 105A was synthesized following the procedure for Example 1A, substituting 2-(thiophen-2-yl)pyrrolidine for benzyl amine.

### Example 105B

### 6-pyridin-4-yl-2-(2-thien-2-ylpyrrolidin-1-yl)-1,3-benzothiazole

The TFA salt of the title compound (0.130 g) was synthesized following the procedure from Example 1B, substituting Example 105A for Example 1A. ¹H NMR (300 MHz, METHANOL-d₄) δ ppm 8.76-8.70 (m, 2H), 8.34 (dd, J=1.9, 7.7, 3H), 7.95 (dd, J=2.1, 8.6, 1H), 7.66 (d, J=8.7, 1H), 7.33 (dd, J=1.3, 5.1, 1H), 7.09 (dd, J=1.8, 2.7, 1H), 6.99 (dd, J=3.6, 5.1, 1H), 5.37 (d, J=5.5, 1H), 4.04-3.92 (m, 1H), 3.84 (dd, J=9.0, 17.0, 1H), 2.67-2.50 (m, 1H), 2.37-2.11 (m, 3H).

### Example 106

### 2-[2-(4-fluorophenyl)pyrrolidin-1-yl]-6-pyridin-4-yl-1,3-benzothiazole

### Example 106A

Example 106A was synthesized following the procedure for Example 1A, substituting 2-(4-fluorophenyl)pyrrolidine for benzyl amine.

### Example 106B

### 2-[2-(4-fluorophenyl)pyrrolidin-1-yl]-6-pyridin-4-yl-1,3-benzothiazole

The TFA salt of the title compound (0.011 g) was synthesized following the procedure from Example 1B, substituting Example 106A for Example 1A. ¹H NMR (500 MHz, METHANOL-d₄) δ ppm 8.72 (d, J=6.7, 2H), 8.32 (d, J=6.0, 3H), 7.94 (dd, J=2.1, 8.6, 1H), 7.63 (d, J=8.6, 1H), 7.35-7.28 (m, 2H), 7.09 (t, J=8.8, 2H), 5.21-4.97 (m, 1H), 4.14-3.84 (m, 2H), 2.65 (s, 1H), 2.27-1.90 (m, 3H).

### Example 107

### 2-(3-phenylpyrrolidin-1-yl)-6-pyridin-4-yl-1,3-benzothiazole

### Example 107A

Example 107A was synthesized following the procedure for Example 1A, substituting 3-phenylpyrrolidine for benzyl amine.

### Example 107B

### 2-(3-phenylpyrrolidin-1-yl)-6-pyridin-4-yl-1,3-benzothiazole

The TFA salt of the title compound (0.059 g) was synthesized following the procedure from Example 1B, substituting Example 107A for Example 1A. ¹H NMR (300 MHz, METHANOL-d₄) δ ppm 8.75 (d, J=7.0, 2H), 8.44 (d, J=2.0, 1H), 8.37 (d, J=7.0, 2H), 8.00 (dd, J=2.0, 8.6, 1H), 7.67 (d, J=8.6, 1H), 7.42-7.24 (m, 5H), 4.10 (s, 1H), 3.89 (s, 1H), 3.81-3.65 (m, 4H), 2.67-2.48 (m, 1H), 2.40-2.23 (m, 1H).

### Example 108

### 2-[2-(5-chlorothien-2-yl)pyrrolidin-1-yl]-6-pyridin-4-yl-1,3-benzothiazole

### Example 108A

Example 108A was synthesized following the procedure for Example 1A, substituting 2-(5-chlorothiophen-2-yl)pyrrolidine for benzyl amine.

### Example 108B

### 2-[2-(5-chlorothien-2-yl)pyrrolidin-1-yl]-6-pyridin-4-yl-1,3-benzothiazole

The TFA salt of the title compound (0.012 g) was synthesized following the procedure from Example 1B, substituting Example 108A for Example 1A. ¹H NMR (300 MHz, METHANOL-d₄) δ ppm 8.74 (d, J=7.0, 2H), 8.36 (dd, J=4.5, 11.3, 3H), 8.00-7.93 (m, 1H), 7.68 (d, J=8.6, 1H), 6.92 (d, J=3.3, 1H), 6.86 (d, J=3.9, 1H), 5.31-5.26 (m, 1H), 3.99-3.80 (m, 2H), 2.60-2.50 (m, 1H), 2.27-2.11 (m, 3H).

### Example 109

### 2-[2-(3-methoxyphenyl)pyrrolidin-1-yl]-6-pyridin-4-yl-1,3-benzothiazole

### Example 109A

Example 109A was synthesized following the procedure for Example 1A, substituting 2-(3-methoxyphenyl)pyrrolidine for benzyl amine.

### Example 109B

### 2-[2-(3-methoxyphenyl)pyrrolidin-1-yl]-6-pyridin-4-yl-1,3-benzothiazole

The TFA salt of the title compound (0.070 g) was synthesized following the procedure from Example 1B, substituting Example 109A for Example 1A. ¹H NMR (300 MHz, METHANOL-d₄) δ ppm 8.71 (d, J=7.0, 2H), 8.33-8.26 (m, 3H), 7.93 (dd, J=2.1, 8.7, 1H), 7.63 (d, J=8.6, 1H), 7.32-7.22 (m, 1H), 6.85 (dd, J=2.9, 6.0, 3H), 5.02 (s, 1H), 4.10-3.88 (m, 2H), 3.78 (s, 3H), 2.68-2.52 (m, 1H), 2.25-1.99 (m, 3H).

### Example 110

### 6-pyridin-4-yl-2-[2-(1,3-thiazol-4-yl)pyrrolidin-1-yl]-1,3-benzothiazole

### Example 110A

Example 110A was synthesized following the procedure for Example 1A, substituting 4-(pyrrolidin-2-yl)thiazole for benzyl amine.

### Example 110B

### 6-pyridin-4-yl-2-[2-(1,3-thiazol-4-yl)pyrrolidin-1-yl]-1,3-benzothiazole

The TFA salt of the title compound (0.0150 g) was synthesized following the procedure from Example 1B, substituting Example 110A for Example 1A. ¹H NMR (300 MHz, METHANOL-d₄) δ ppm 8.75 (d, J=7.0, 2H), 8.44-8.33 (m, 3H), 7.96 (dd, J=2.1, 8.6, 1H), 7.79 (d, J=3.2, 1H), 7.66 (d, J=8.7, 1H), 7.54 (d, J=3.3, 1H), 5.53 (d, J=8.4, 1H), 4.05-3.94 (m, 1H), 3.79 (dd, J=8.2, 17.4, 1H), 2.69-2.57 (m, 1H), 2.37-2.18 (m, 3H).

### Example 111

### 2-(2-benzylpyrrolidin-1-yl)-6-pyridin-4-yl-1,3-benzothiazole

### Example 111A

### Example 111A was synthesized following the procedure for Example 1A, substituting 2-benzylpyrrolidine for benzyl amine.

### Example 111B

### 2-(2-benzylpyrrolidin-1-yl)-6-pyridin-4-yl-1,3-benzothiazole

The TFA salt of the title compound (0.150 g) was synthesized following the procedure from Example 1B, substituting Example 111A for Example 1A. ¹H NMR (300 MHz, METHANOL-d₄) δ ppm 8.83-8.69 (m, 2H), 8.50-8.32 (m, 3H), 8.01 (dd, J=2.1, 8.7, 1H), 7.71 (d, J=8.6, 1H), 7.40-7.15 (m, 5H), 4.34 (s, 1H), 3.69-3.47 (m, 2H), 3.25-3.13 (m, 1H), 2.94 (dd, J=8.7, 13.4, 1H), 2.17-1.88 (m, 4H).

### Example 112

### 2-[2-(5-methylthien-2-yl)pyrrolidin-1-yl]-6-pyridin-4-yl-1,3-benzothiazole

### Example 112A

Example 112A was synthesized following the procedure for Example 1A, substituting 2-(5-methylthien-2-yl)pyrrolidine for benzyl amine.

### Example 112B

### 2-[2-(5-methylthien-2-yl)pyrrolidin-1-yl]-6-pyridin-4-yl-1,3-benzothiazole

The TFA salt of the title compound (0.181 g) was synthesized following the procedure from Example 1B, substituting Example 112A for Example 1A. ¹H NMR (300 MHz, METHANOL-d₄) δ ppm 8.71 (d, J=6.9, 2H), 8.32 (d, J=2.0, 1H), 8.28 (d, J=6.9, 2H), 7.93 (dd, J=2.1, 8.7, 1H), 7.65 (d, J=8.6, 1H), 6.86 (d, J=3.4, 1H), 6.66-6.60 (m, 1H), 5.23 (d, J=5.1, 1H), 4.00-3.76 (m, 1H), 2.62-2.46 (m, 1H), 2.42 (d, J=1.1, 3H), 2.39-2.25 (m, 1H), 2.25-2.07 (m, 2H).

### Example 113

### 2-[2-(3-fluorophenyl)pyrrolidin-1-yl]-6-pyridin-4-yl-1,3-benzothiazole

### Example 113A

Example 113A was synthesized following the procedure for Example 1A, substituting 2-(3-fluorophenyl)pyrrolidine for benzyl amine.

### Example 113B

### 2-[2-(3-fluorophenyl)pyrrolidin-1-yl]-6-pyridin-4-yl-1,3-benzothiazole

The TFA salt of the title compound (0.180 g) was synthesized following the procedure from Example 1B, substituting Example 113A for Example 1A. ¹H NMR (300 MHz, METHANOL-d₄) δ ppm 8.70 (d, J=6.9, 2H), 8.28 (dd, J=4.4, 14.3, 3H), 7.91 (dd, J=2.1, 8.6, 1H), 7.63 (d, J=8.6, 1H), 7.38 (td, J=5.9, 7.9, 1H), 7.20-6.92 (m, 3H), 5.09 (s, 1H), 4.14-3.81 (m, 2H), 2.62 (ddd, J=7.8, 12.1, 16.5, 1H), 2.28-1.95 (m, 3H).

### Example 114

### 2-[2-(3-chlorophenyl)pyrrolidin-1-yl]-6-pyridin-4-yl-1,3-benzothiazole

### Example 114A

Example 114A was synthesized following the procedure for Example 1A, substituting 2-(3-chlorophenyl)pyrrolidine for benzyl amine.

### Example 114B

### 2-[2-(3-chlorophenyl)pyrrolidin-1-yl]-6-pyridin-4-yl-1,3-benzothiazole

The TFA salt of the title compound (0.205 g) was synthesized following the procedure from Example 1B, substituting Example 114A for Example 1A. ¹H NMR (300 MHz, METHANOL-d₄) δ ppm 8.73 (d, J=7.0, 2H), 8.37-8.30 (m, 3H), 7.95 (dd, J=2.0, 8.6, 1H), 7.64 (d, J=8.6, 1H), 7.40-7.20 (m, 4H), 5.08 (s, 1H), 4.12-3.86 (m, 2H), 2.63-2.55 (m, 1H), 2.24-1.99 (m, 3H).

### Example 115

### 6-pyridin-4-yl-2-[(2S)-2-thien-2-ylpyrrolidin-1-yl]-1,3-benzothiazole

### Example 116

### 6-pyridin-4-yl-2-[(2R)-2-thien-2-ylpyrrolidin-1-yl]-1,3-benzothiazole

The TFA salts of Examples 115(0.001 g) and 116(0.001 g) were isolated by chiral separation of example 105(0.01 g) using a semi-preparative HPLC on a 25 cm ChiralPak AD with 1 cm i.d. and a 5 μm particle size. An isocratic gradient of hexanes/ethanol/methanol/diethylamine (30/35/35/0.1) was used, at a flow rate of 10 mL/min, a pressure of 100 bar and a temeperature of 25° C. The sample was injected as solutions in methanol. Fractions (with retention times 9.03 min and 14.5 min respectively) were collected based upon UV signal threshold and selected fractions subsequently analyzed by flow injection analysis mass spectrometry using positive APCI ionization on a Finnigan LCQ using 70:30 methanol: 10 mM NH₄OH(aq) at a flow rate of 0.8 mL/min. Loop-injection mass spectra were acquired using a Finnigan LCQ running LCQ Navigator 1.2 software and a Gilson 215 liquid handler for fraction injection controlled by an Abbott developed Visual Basic application.

### Example 117

### 3-[1-(6-pyridin-4-yl-1,3-benzothiazol-2-yl)pyrrolidin-2-yl]phenol

### Example 117A

Example 117A was synthesized following the procedure for Example 1A, substituting 3-(pyrrolidin-2-yl)phenol for benzyl amine.

### Example 117B

### 3-[1-(6-pyridin-4-yl-1,3-benzothiazol-2-yl)pyrrolidin-2-yl]phenol

The TFA salt of the title compound (0.157 g) was synthesized following the procedure from Example 1B, substituting Example 117A for Example 1A. ¹H NMR (300 MHz, METHANOL-d₄) δ ppm 8.77-8.70 (m, 2H), 8.33 (d, J=7.0, 3H), 7.96 (dd, J=2.0, 8.7, 1H), 7.65 (d, J=8.6, 1H), 7.23-7.13 (m, 1H), 6.72 (ddd, J=3.7, 4.6, 10.9, 3H), 4.98 (s, 1H), 4.11-3.88 (m, 2H), 2.67-2.52 (m, 1H), 2.31-1.99 (m, 3H).

### Example 118

### 3-{1-[6-(1H-pyrrolo[2,3-b]pyridin-4-yl)-1,3-benzothiazol-2-yl]pyrrolidin-2-yl}phenol

Example 118 (0.038 g) was synthesized following the procedure from Example 6B, substituting Example 117A for Example 2A. ¹H NMR (300 MHz, METHANOL-d₄) δ ppm 8.36 (d, J=6.1, 1H), 8.20 (d, J=1.8, 1H), 7.87 (dd, J=1.9, 8.5, 1H), 7.73-7.64 (m, 2H), 7.59 (d, J=6.1, 1H), 7.24-7.15 (m, 1H), 6.99 (d, J=3.6, 1H), 6.79 (d, J=7.8, 1H), 6.72 (d, J=7.2, 1H), 4.13-3.90 (m, 2H), 2.70-2.53 (m, 1H).

### Example 119

### 2-{2-[3-(2-morpholin-4-ylethoxy)phenyl]pyrrolidin-1-yl}-6-pyridin-4-yl-1,3-benzothiazole

### Example 119A

Example 117A (0.2 g, 0.53 mmol) was dissolved in N,N-dimethyl formamide (1 mL) and treated with 60% NaH(0.020 g, 0.53 mmol) and stirred at 25° C. for 1 hour. To the reaction mixture was added 4-(2-chloroethyl)morpholine (0.077 g, 0.53 mmol) and heated at 75° C. for an additional 12 hours. The cooled reaction mixture was diluted with dichloromethane (5 mL) and washed with saturated NaHCO₃ (2×10 mL) and water (2×10 mL). The organic layer was separated and concentrated to a yellow oil which was used in the next step without further purification.

### Example 119B

### 2-{2-[3-(2-morpholin-4-ylethoxy)phenyl]pyrrolidin-1-yl}-6-pyridin-4-yl-1,3-benzothiazole

The TFA salt of the title compound (0.049 g) was synthesized following the procedure from Example 1B, substituting Example 119A for Example 1A. ¹H NMR (300 MHz, METHANOL-d₄) δ ppm 8.74 (d, J=7.0, 2H), 8.33 (d, J=7.1, 3H), 7.95 (dd, J=2.1, 8.6, 1H), 7.63 (d, J=8.7, 1H), 7.34 (t, J=7.9, 1H), 6.95 (d, J=8.1, 3H), 5.19-5.01 (m, 1H), 4.37 (s, 2H), 4.02 (s, 7H), 3.60 (dd, J=12.4, 17.3, 5H), 2.61 (s, 1H), 2.27-1.98 (m, 3H).

### Example 120

### 2-{2-[3-(2-piperidin-1-ylethoxy)phenyl]pyrrolidin-1-yl}-6-pyridin-4-yl-1,3-benzothiazole

### Example 120A

### Example 120A (0.249 g) was synthesized following the procedure from Example 119A, substituting 1-(2-chloroethyl)piperidine for 4-(2-chloroethyl)morpholine.

### Example 120B

### 2-{2-[3-(2-piperidin-1-ylethoxy)phenyl]pyrrolidin-1-yl}-6-pyridin-4-yl-1,3-benzothiazole

The TFA salt of the title compound (0.006 g) was synthesized following the procedure from Example 1B, substituting Example 120A for Example 1A. ¹H NMR (500 MHz, METHANOL-d₄) δ ppm 8.73 (d, J=6.8, 2H), 8.32 (d, J=6.9, 3H), 7.95 (dd, J=2.0, 8.6, 1H), 7.63 (d, J=8.6, 1H), 7.34 (t, J=7.9, 1H), 6.99-6.91 (m, 3H), 5.20-4.96 (m, 1H), 4.35 (dd, J=4.8, 10.3, 2H), 4.04 (s, 2H), 3.67-3.48 (m, 4H), 3.04 (t, J=12.4, 2H), 2.63 (d, J=22.8, 1H), 2.27-1.86 (m, 6H).

### Example 121

### 3-[1-(6-pyrimidin-4-yl-1,3-benzothiazol-2-yl)pyrrolidin-2-yl]phenol

The TFA salt of the title compound (0.012 g) was synthesized following the procedure from Example 86, substituting Example 117A for Example 12A. ¹H NMR (300 MHz, METHANOL-d₄) δ ppm 9.16 (s, 1H), 8.76 (d, J=5.7, 1H), 8.58 (s, 1H), 8.26 (dd, J=1.7, 8.6, 1H), 8.02 (d, J=5.7, 1H), 7.63 (d, J=8.6, 2H), 7.27-7.16 (m, 1H), 6.84-6.75 (m, 2H), 5.04 (s, 1H), 4.04 (d, J=26.7, 2H), 2.61 (d, J=7.5, 1H), 2.27-2.11 (m, 3H).

### Example 122

### 3-{1-[6-(2-fluoropyridin-4-yl)-1,3-benzothiazol-2-yl]pyrrolidin-2-yl}phenol

The TFA salt of the title compound (0.142 g) was synthesized following the procedure from Example 1B, substituting Example 117A for Example 1A and substituting 2-fluoropyridin-4-ylboronic acid for 4-(4,4,5,5-tetramethyl-1,3,2-dioxaborolan-2-yl)pyridine.

¹H NMR (300 MHz, METHANOL-d₄) δ ppm 8.21 (d, J=5.4, 1H), 8.11 (d, J=1.8, 1H), 7.79 (dd, J=1.9, 8.5, 1H), 7.64-7.58 (m, 2H), 7.35 (s, 1H), 7.24-7.15 (m, 1H), 6.82-6.75 (m, 1H), 6.75 (s, 2H), 4.99 (s, 1H), 4.13-3.89 (m, 2H), 2.70-2.54 (m, 1H), 2.30-2.06 (m, 3H).

### Example 123

### N-(3-fluorobenzyl)-5-pyridin-4-yl-1,3-benzothiazol-2-amine

### Example 123A

Example 123A (0.26 g) was synthesized following the procedure for Example 1A, substituting 5-bromo-2-chlorobenzo[d]thiazole for 6-bromo-2-chlorobenzo[d]thiazole and (3-fluorophenyl)methanamine for benzyl amine.

### Example 123B

### N-(3-fluorobenzyl)-5-pyridin-4-yl-1,3-benzothiazol-2-amine

The TFA salt of the title compound (0.142 g) was synthesized following the procedure from Example 1B, substituting Example 123A for Example 1A. ¹H NMR (300 MHz, METHANOL-d₄) δ 8.77 (d, J=6.8, 2H), 8.33-8.26 (m, 2H), 7.96 (d, J=1.9, 1H), 7.86 (d, J=8.2, 1H), 7.65 (dd, J=1.9, 8.3, 1H), 7.37 (td, J=5.9, 7.9, 1H), 7.19 (dd, J=8.8, 22.8, 2H), 7.01 (td, J=2.3, 8.3, 1H).

### Example 124

### N-benzyl-6-pyridin-4-yl-1,3-benzoxazol-2-amine

### Example 124A

Example 124A (0.05 g) was synthesized following the procedure for Example 1A, substituting 2,6-dichlorobenzo[d]oxazole for 6-bromo-2-chlorobenzo[d]thiazole

### Example 124B

### N-benzyl-6-pyridin-4-yl-1,3-benzoxazol-2-amine

The TFA salt of the title compound (0.001 g) was synthesized following the procedure from Example 1B, substituting Example 124A for Example 1A. ¹A NMR (300 MHz, METHANOL-d₄) δ ppm 2.19 (s, 1H) 4.64 (s, 2H) 7.23-7.50 (m, 6H) 7.82 (dd, J=8.31, 1.87 Hz, 1H) 7.94 (d, J=1.36 Hz, 1H) 8.20 (d, J=6.78 Hz, 2H) 8.69 (d, J=6.44 Hz, 2H).

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

We claim:

1. A compound having formula (I) 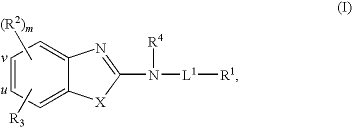
or a pharmaceutically acceptable salt, solvate, prodrug, salt of a prodrug, or combination thereof, wherein
X is S or O;
R⁴ is hydrogen, alkyl, —(C₂₋₆ alkylene)-OR^{4g}, —(C₂₋₆ alkylene)-NR^{4k}R^{4m}, or haloalkyl;
L¹ is (CR^{p}R^{q})ₙ, (CR^{p}R^{q})ᵣ—X¹, or (CR^{p}R^{q})ᵣ—X¹—(CR^{p}R^{q})ₙ, wherein the (CR^{p}R^{q})ᵣ group of the (CR^{p}R^{q})ᵣ—X¹ and the (CR^{p}R^{q})ᵣ—X¹—(CR^{p}R^{q})ₙ is connected to N(R⁴) of formula (I),
X¹ is N(R⁵), O, or S; and
R¹ is —Si(R^{1a})₃, aryl, heteroaryl, heterocycle, cycloalkyl, or cycloalkenyl; each of the aryl, heteroaryl, heterocycle, cycloalkyl, and cycloalkenyl is optionally substituted with 1, 2, 3, 4, or 5 substituents as represented by R^{y};
or
L¹-R¹ together is formula (i) 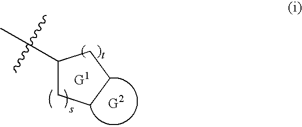
wherein s is 0, 1, 2, or 3;
t is 0, 1, 2, or 3; with the proviso that s and t are not both 0;
one or two CH₂ units of ring G¹ is optionally replaced by NH, O, N(O), S, S(O), or S(O)₂, ring G² is phenyl or monocyclic heteroaryl; and G¹ and G² are each independently unsubstituted or substituted with 1, 2, 3, 4, or 5 substituents as represented by R^{y′};
or
N(R⁴)-L¹-R¹ together is formula (ii) 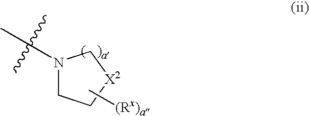
wherein
a′ is 1 or 2;
X² is CH₂, S, S(O), S(O)₂, O, or N(R⁶) wherein R⁶ is hydrogen, alkyl, —C(O)O(alkyl), —C(O)O(benzyl), or benzyl; provided that when X² is other than CH₂, then a′ is 2;
a″ is 0, 1, or 2; and
each occurrence of R^{x} represents an optional substituent on any substitutable carbon or nitrogen atom of formula (ii), and is independently alkyl, haloalkyl, aryl, arylalkyl, heteroaryl, or heteroarylalkyl; wherein each of the aryl and heteroaryl moieties, by itself or as part of the substituent, is independently unsubstituted or substituted with 1, 2, 3, or 4 R^{y} groups;
R^{1a}, at each occurrence, is independently alkyl, aryl, or arylalkyl wherein the aryl moiety, by itself of as part of the substituent, is independently unsubstituted or substituted with 1, 2, 3, 4, or 5 substituents selected from the group consisting of alkyl, haloalkyl, and halogen;
n is 1, 2, 3, 4, 5, or 6;
r is 2, 3, 4, 5, or 6;
R^{p} and R^{q}, at each occurrence, are each independently hydrogen, alkyl, haloalkyl, —OR^{gp}, —NR^{kp}R^{mp}, —(C₁₋₆ alkylene)-OR^{gp}, —(C₁₋₆ alkylene)-NR^{kp}R^{mp}, or —(C₁₋₆ alkylene)-N—OR^{gp};
each R² represents an optional substituent on the benzene ring of formula (I), and at each occurrence, is independently selected from the group consisting of alkyl, alkenyl, alkynyl, halogen, NO₂, CN, haloalkyl, OR^{a}, —O—(C₂₋₆ alkylene)-NR^{k}R^{m}, OC(O)R^{a}, NR^{a}R^{b}, —N(R^{a})—(C₂₋₆ alkylene)-NR^{k}R^{m}, SR^{a}, S(O)R^{c}, S(O)₂R^{c}, S(O)₂NR^{a}R^{b}, C(O)R^{a}, C(O)OR^{a}, C(O)NR^{a}R^{b}, —(C₁₋₆ alkylene)-NO₂, —(C₁₋₆ alkylene)-CN, —(C₁₋₆ alkylene)-OR^{a}, —(C₁₋₆ alkylene)-OC(O)R^{a}, —(C₁₋₆ alkylene)-NR^{a}R^{b}, —(C₁₋₆ alkylene)-SR^{a}, —(C₁₋₆ alkylene)-S(O)R^{c}, —(C₁₋₆ alkylene)-S(O)₂R^{c}, —(C₁₋₆ alkylene)-S(O)₂NR^{a}R^{b}, —(C₁₋₆ alkylene)-C(O)R^{a}, —(C₁₋₆ alkylene)-C(O)OR^{a}, and —(C₁₋₆ alkylene)-C(O)NR^{a}R^{b};
m is 0, 1, 2, or 3;
R³ is heterocycle or heteroaryl, each of which is attaching to the benzene ring of formula (I) via position u or v, and is optionally substituted with 1, 2, 3, 4, or 5 substituents as represented by T, wherein each T is independently selected from the group consisting of alkyl, alkenyl, alkynyl, halogen, NO₂, CN, haloalkyl, OR^{d}, OC(O)R^{d}, NR^{d}R^{e}, SR^{d}, S(O)R^{f}, S(O)₂R^{f}, S(O)₂NR^{d}R^{e}, C(O)R^{d}, C(O)OR^{d}, C(O)NR^{d}R^{e}, —(C₁₋₆ alkylene)-NO₂, —(C₁₋₆ alkylene)-CN, —(C₁₋₆ alkylene)-OR^{d}, —(C₁₋₆ alkylene)-OC(O)R^{d}, —(C₁₋₆ alkylene)-NR^{d}R^{e}, —(C₁₋₆ alkylene)-SR^{d}, —(C₁₋₆ alkylene)-S(O)R^{f}, —(C₁₋₆ alkylene)-S(O)₂R^{f}, —(C₁₋₆ alkylene)-S(O)₂NR^{d}R^{e}, —(C₁₋₆ alkylene)-C(O)R^{d}, —(C₁₋₆ alkylene)-C(O)OR^{d}, and —(C₁₋₆ alkylene)-C(O)NR^{d}R^{e};
R^{y} and R^{y′} at each occurrence, are each independently selected from the group consisting of alkyl, alkenyl, alkynyl, halogen, NO₂, CN, oxo, haloalkyl, OR^{g}, —O—(C₁₋₆ alkylene)-Si(R^{1a})₃, —O—(C₂₋₆ alkylene)-NR^{k}R^{m}, —OC(O)R^{g}, —NR^{k}R^{m}, —N(R^{g})—(C₂₋₆ alkylene)-NR^{k}R^{m}, —N(R^{g})S(O)₂R^{j}, —N(R^{g})C(O)OR^{g}, —SR^{g}, —S(O)R^{j}, —S(O)₂R^{j}, —S(O)₂NR^{k}R^{m}, —C(O)R^{g}, —C(O)OR^{g}, —C(O)NR^{k}R^{m}, —C(O)N(R^{g})(C₂₋₆ alkylene-NR^{k}R^{m}), —C(O)N(R^{g})(C₂₋₆ alkylene-OR_{g}), —(C₁₋₆ alkylene)-NO₂, —(C₁₋₆ alkylene)-CN, —(C₁₋₆ alkylene)-OR^{g}, —(C₁₋₆ alkylene)-OC(O)R^{g}, —(C₁₋₆ alkylene)-NR^{k}R^{m}, —(C₁₋₆ alkylene)-SR^{g}, —(C₁₆ alkylene)-S(O)R^{j}, —(C₁₋₆ alkylene)-S(O)₂R^{j}, —(C₁₋₆ alkylene)-S(O)NR^{k}R^{m}, —(C₁₋₆ alkylene)-C(O)R^{g}, —(C₁₋₆ alkylene)-C(O)OR^{g}, and —(C₁₋₆ alkylene)-C(O)NR^{k}R^{m};
w is 1, 2, or 3;
R^{a}, R^{b}, R^{d}, R^{e}, R^{g}, R^{4g}, and R^{gp}, at each occurrence, are each independently hydrogen, alkyl, or haloalkyl;
R^{c}, R^{f}, and R^{j}, at each occurrence, are each independently alkyl or haloalkyl;
R^{k}, R^{m}, R^{4k}, R^{4m}, R^{kp}, and R^{mp}, at each occurrence, are each independently hydrogen, alkyl, or haloalkyl;
R^{k} and R^{m}, R^{4k} and R^{4m}, R^{kp} and R^{mp}, together with the nitrogen atom to which they are attached, optionally form a 5- or 6-membered monocyclic heterocycle, said monocyclic heterocycle optionally contains 0 or 1 additional heteroatom, and is optionally substituted with 1, 2, 3, or 4 substituents independently selected from the group consisting of oxo, alkyl, and haloalkyl; and
R⁵ is hydrogen, alkyl, haloalkyl, alkoxyalkyl, haloalkoxyalkyl, or hydroxyalkyl;
with the proviso that when X is O then R³ is not pyrimidin-5-yl; and with the further proviso that when X is S, L¹ is (CR^{p}R^{q})ₙ wherein n is 1, R^{p} and R^{q} are hydrogen, and R¹ ^{_{optionally substituted phenyl, then R}}³ is not imidazolyl.

2. The compound of claim 1 or a pharmaceutically acceptable salt thereof, wherein X is S.

3. The compound of claim 1 or a pharmaceutically acceptable salt thereof, wherein X is S, and L¹ is (CR^{p}R^{q})ₙ or (CR^{p}R^{q})ᵣ—X¹.

4. The compound of claim 1 or a pharmaceutically acceptable salt thereof, wherein X is S, L¹ is (CR^{p}R^{q})ₙ or (CR^{p}R^{q})ᵣ—X¹, and R¹ is —Si(R^{1a})₃, optionally substituted aryl, and optionally substituted heterocycle.

5. The compound of claim 1 or a pharmaceutically acceptable salt thereof, wherein X is S, L¹ is (CR^{p}R^{q})ₙ or (CR^{p}R^{q})ᵣ—X¹, and R¹ is optionally substituted phenyl.

6. The compound of claim 1 or a pharmaceutically acceptable salt thereof, wherein X is S, L¹ is (CR^{p}R^{q})ₙ or (CR^{p}R^{q})ᵣ—X¹, R¹ is optionally substituted phenyl, and R³ is optionally substituted pyridinyl.

7. The compound of claim 1 or a pharmaceutically acceptable salt thereof, wherein X is O, L¹ is (CR^{p}R^{q})ₙ or (CR^{p}R^{q})ᵣ—X¹, R¹ is optionally substituted phenyl, and R³ is optionally substituted pyridinyl.

8. The compound of claim 1 or a pharmaceutically acceptable salt thereof, wherein L¹-R¹ together is formula (i)

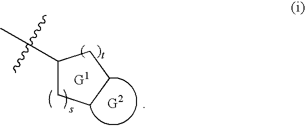

9. The compound of claim 1 or a pharmaceutically acceptable salt thereof, wherein N(R⁴)-L¹-R¹ together is formula (ii)

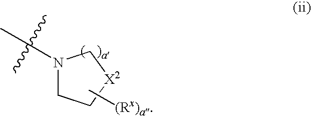

10. The compound of claim 1 having formula (Ia) or a pharmaceutically acceptable salt thereof

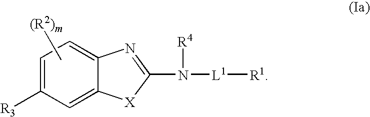

11. The compound of claim 10 or a pharmaceutically acceptable salt thereof wherein X is S.

12. The compound of claim 10 or a pharmaceutically acceptable salt thereof wherein X is S, L¹ is (CR^{p}R^{q})ₙ or (CR^{p}R^{q})ᵣ—X¹, and R¹ is —Si(R^{1a})₃, optionally substituted aryl, and optionally substituted heterocycle.

13. The compound of claim 10 or a pharmaceutically acceptable salt thereof wherein X is S, L¹ is (CR^{p}R^{q})ₙ or (CR^{p}R^{q})ᵣ—X¹, and R¹ is optionally substituted phenyl.

14. The compound of claim 10 or a pharmaceutically acceptable salt thereof wherein X is S, L¹ is (CR^{p}R^{q})ₙ or (CR^{p}R^{q})ᵣ—X¹, R¹ is optionally substituted phenyl, and R³ is optionally substituted pyridinyl.

15. The compound of claim 10 or a pharmaceutically acceptable salt thereof wherein X is S, and N(R⁴)-L¹-R¹ together is formula (ii).

16. The compound of claim 1 or a pharmaceutically acceptable salt thereof, selected from the group consisting of
N-benzyl-6-pyridin-4-yl-1,3-benzothiazol-2-amine;
N-(2-phenylethyl)-6-pyridin-4-yl-1,3-benzothiazol-2-amine;
N-(1-naphthylmethyl)-6-pyridin-4-yl-1,3-benzothiazol-2-amine;
2-phenyl-2-[(6-pyridin-4-yl-1,3-benzothiazol-2-yl)amino]ethanol;
N-(3-phenylpropyl)-6-pyridin-4-yl-1,3-benzothiazol-2-amine;
N-(2-phenylethyl)-6-(1H-pyrrolo[2,3-b]pyridin-4-yl)-1,3-benzothiazol-2-amine;
N-[3,5-bis(trifluoromethyl)benzyl]-6-pyridin-4-yl-1,3-benzothiazol-2-amine;
N-(3,4-dihydro-1H-isochromen-3-ylmethyl)-6-pyridin-4-yl-1,3-benzothiazol-2-amine;
N-(1,3-benzodioxol-5-ylmethyl)-6-pyridin-4-yl-1,3-benzothiazol-2-amine;
2-methoxy-5-{[(6-pyridin-4-yl-1,3-benzothiazol-2-yl)amino]methyl}phenol;
1-phenyl-2-[(6-pyridin-4-yl-1,3-benzothiazol-2-yl)amino]ethanol;
N-(2,3-dihydro-1,4-benzodioxin-5-ylmethyl)-6-pyridin-4-yl-1,3-benzothiazol-2-amine;
2-[benzyl(6-pyridin-4-yl-1,3-benzothiazol-2-yl)amino]ethanol;
N-[(1R)-1-(3-methoxyphenyl)ethyl]-6-pyridin-4-yl-1,3-benzothiazol-2-amine;
N-benzyl-6-(3-fluoropyridin-4-yl)-1,3-benzothiazol-2-amine;
N-(3,4-dihydro-2H-1,5-benzodioxepin-6-ylmethyl)-6-pyridin-4-yl-1,3-benzothiazol-2-amine;
N-(2,3-dihydro-1,4-benzodioxin-5-ylmethyl)-6-(1H-pyrrolo[2,3-b]pyridin-4-yl)-1,3-benzothiazol-2-amine;
N-(2-ethoxybenzyl)-6-pyridin-4-yl-1,3-benzothiazol-2-amine;
N-[2-(methylthio)benzyl]-6-pyridin-4-yl-1,3-benzothiazol-2-amine;
N-[2-(difluoromethoxy)benzyl]-6-pyridin-4-yl-1,3-benzothiazol-2-amine;
N-[3-(difluoromethoxy)benzyl]-6-pyridin-4-yl-1,3-benzothiazol-2-amine;
N-[3-fluoro-5-(trifluoromethyl)benzyl]-6-pyridin-4-yl-1,3-benzothiazol-2-amine;
N-[2-(2-methylphenyl)ethyl]-6-pyridin-4-yl-1,3-benzothiazol-2-amine;
2-[methyl(6-pyridin-4-yl-1,3-benzothiazol-2-yl)amino]-1-phenylethanol;
N-[(1S)-1-phenylethyl]-6-pyridin-4-yl-1,3-benzothiazol-2-amine;
N-[(1R)-1-phenylethyl]-6-pyridin-4-yl-1,3-benzothiazol-2-amine;
N-(2-phenoxyethyl)-6-pyridin-4-yl-1,3-benzothiazol-2-amine;
N-[(1R)-1-(1-naphthyl)ethyl]-6-pyridin-4-yl-1,3-benzothiazol-2-amine;
N-[(1S)-1-(1-naphthyl)ethyl]-6-pyridin-4-yl-1,3-benzothiazol-2-amine;
N-[(1R)-1-phenylpropyl]-6-pyridin-4-yl-1,3-benzothiazol-2-amine;
N-(3-fluorobenzyl)-6-(pyridin-4-yl)benzo[d]thiazol-2-amine;
N-[(1R)-2,3-dihydro-1H-inden-1-yl]-6-pyridin-4-yl-1,3-benzothiazol-2-amine;
N-[(1S)-2,3-dihydro-1H-inden-1-yl]-6-pyridin-4-yl-1,3-benzothiazol-2-amine;
6-pyridin-4-yl-N-[3-(trimethylsilyl)propyl]-1,3-benzothiazol-2-amine;
N-benzyl-6-(1H-indazol-5-yl)-1,3-benzothiazol-2-amine;
6-(1H-indazol-5-yl)-N-(3-phenylpropyl)-1,3-benzothiazol-2-amine;
N-[(1R)-1-(2-naphthyl)ethyl]-6-pyridin-4-yl-1,3-benzothiazol-2-amine;
N-(2,3-dimethoxybenzyl)-6-pyridin-4-yl-1,3-benzothiazol-2-amine;
N-(2,5-dimethoxybenzyl)-6-pyridin-4-yl-1,3-benzothiazol-2-amine;
(2R)-2-phenyl-2-[(6-pyridin-4-yl-1,3-benzothiazol-2-yl)amino]ethanol;
N-(3-isopropoxybenzyl)-6-pyridin-4-yl-1,3-benzothiazol-2-amine;
N-[(2,2-dimethyl-2,3-dihydro-1-benzofuran-7-yl)methyl]-6-pyridin-4-yl-1,3-benzothiazol-2-amine;
2-(4-methyl-2-phenylpiperazin-1-yl)-6-pyridin-4-yl-1,3-benzothiazole;
N-(2,3-dichlorobenzyl)-6-pyridin-4-yl-1,3-benzothiazol-2-amine;
(3R)-3-phenyl-3-[(6-pyridin-4-yl-1,3-benzothiazol-2-yl)amino]propan-1-ol;
N-(2-fluorobenzyl)-6-pyridin-4-yl-1,3-benzothiazol-2-amine;
N-(2,3-difluorobenzyl)-6-pyridin-4-yl-1,3-benzothiazol-2-amine;
N-(2,3-dimethylbenzyl)-6-pyridin-4-yl-1,3-benzothiazol-2-amine;
N-[1-(2,3-dihydro-1,4-benzodioxin-5-yl)ethyl]-6-pyridin-4-yl-1,3-benzothiazol-2-amine;
N-[1-(2-chlorophenyl)ethyl]-6-pyridin-4-yl-1,3-benzothiazol-2-amine;
(2S)-2-phenyl-2-[(6-pyridin-4-yl-1,3-benzothiazol-2-yl)amino]ethanol;
3-(3-methoxyphenyl)-3-[(6-pyridin-4-yl-1,3-benzothiazol-2-yl)amino]propan-1-ol;
N-(3-methylbenzyl)-6-pyridin-4-yl-1,3-benzothiazol-2-amine;
(1S,2R)-1-[(6-pyridin-4-yl-1,3-benzothiazol-2-yl)amino]indan-2-ol;
N-[3-(hydroxyamino)-1-(3-methoxyphenyl)propyl]-6-pyridin-4-yl-1,3-benzothiazol-2-amine;
(1R,2S)-1-[(6-pyridin-4-yl-1,3-benzothiazol-2-yl)amino]indan-2-ol;
N-[(1R)-1-(2,3-dihydro-1,4-benzodioxin-5-yl)ethyl]-6-pyridin-4-yl-1,3-benzothiazol-2-amine;
N-[(1S)-1-(2,3-dihydro-1,4-benzodioxin-5-yl)ethyl]-6-pyridin-4-yl-1,3-benzothiazol-2-amine;
N-(3-chlorobenzyl)-6-pyridin-4-yl-1,3-benzothiazol-2-amine;
N-[1-(3-fluorophenyl)ethyl]-6-pyridin-4-yl-1,3-benzothiazol-2-amine;
(3R)-3-[(6-pyridin-4-yl-1,3-benzothiazol-2-yl)amino]indan-5-ol;
N-(3-fluorobenzyl)-6-(3-fluoropyridin-4-yl)-1,3-benzothiazol-2-amine;
(1R,2R)-1-[(6-pyridin-4-yl-1,3-benzothiazol-2-yl)amino]indan-2-ol;
(1S,2S)-1-[(6-pyridin-4-yl-1,3-benzothiazol-2-yl)amino]indan-2-ol;
N-(3-fluorobenzyl)-6-(2-fluoropyridin-4-yl)-1,3-benzothiazol-2-amine;
N-[(1R)-1-(3-fluorophenyl)ethyl]-6-pyridin-4-yl-1,3-benzothiazol-2-amine;
N-(3-fluorobenzyl)-6-(1H-pyrazol-5-yl)-1,3-benzothiazol-2-amine;
N-[(1R)-1-(3-ethoxyphenyl)ethyl]-6-pyridin-4-yl-1,3-benzothiazol-2-amine;
3-{[(6-pyridin-4-yl-1,3-benzothiazol-2-yl)amino]methyl}phenol;
N-(3-aminobenzyl)-6-pyridin-4-yl-1,3-benzothiazol-2-amine;
N-[4-(aminomethyl)benzyl]-6-pyridin-4-yl-1,3-benzothiazol-2-amine;
N-[3-(2-morpholin-4-ylethoxy)benzyl]-6-pyridin-4-yl-1,3-benzothiazol-2-amine;
N-(3-fluorobenzyl)-6-(2-methylpyridin-4-yl)-1,3-benzothiazol-2-amine;
3-({[6-(1H-pyrrolo[2,3-b]pyridin-4-yl)-1,3-benzothiazol-2-yl]amino}methyl)phenol;
N-{3-[2-(dimethylamino)ethoxy]benzyl}-6-pyridin-4-yl-1,3-benzothiazol-2-amine;
6-(3-fluoropyridin-4-yl)-N-[3-(2-morpholin-4-ylethoxy)benzyl]-1,3-benzothiazol-2-amine;
6-[3-(aminomethyl)pyridin-4-yl]-N-(3-fluorobenzyl)-1,3-benzothiazol-2-amine;
N-[3-(2-morpholin-4-ylethoxy)benzyl]-6-(1H-pyrrolo[2,3-b]pyridin-4-yl)-1,3-benzothiazol-2-amine;
N-{3-[3-(dimethylamino)propoxy]benzyl}-6-pyridin-4-yl-1,3-benzothiazol-2-amine;
N-(2,3-dihydro-1,4-benzodioxin-5-ylmethyl)-6-(3-fluoropyridin-4-yl)-1,3-benzothiazol-2-amine;
(2S)-2-{[6-(3-fluoropyridin-4-yl)-1,3-benzothiazol-2-yl]amino}-2-phenylethanol;
3-{[(6-pyridin-4-yl-1,3-benzothiazol-2-yl)(2-pyrrolidin-1-ylethyl)amino]methyl}phenol;
N-[3-(morpholin-4-ylmethyl)benzyl]-6-pyridin-4-yl-1,3-benzothiazol-2-amine;
N-[3-(2-piperidin-1-ylethoxy)benzyl]-6-pyridin-4-yl-1,3-benzothiazol-2-amine;
N-(3-{[(6-pyridin-4-yl-1,3-benzothiazol-2-yl)amino]methyl}phenyl)methanesulfonamide;
N-(2,3-dihydro-1,4-benzodioxin-5-ylmethyl)-6-pyrimidin-4-yl-1,3-benzothiazol-2-amine;
N-[3-(4-methylpiperazin-1-yl)benzyl]-6-pyridin-4-yl-1,3-benzothiazol-2-amine;
N-[3-(2-morpholin-4-ylethoxy)benzyl]-6-pyrimidin-4-yl-1,3-benzothiazol-2-amine;
N-[3-(2-piperidin-1-ylethoxy)benzyl]-6-pyrimidin-4-yl-1,3-benzothiazol-2-amine;
N-(3-fluorobenzyl)-6-pyrimidin-4-yl-1,3-benzothiazol-2-amine;
6-pyrimidin-4-yl-N-[3-(2-pyrrolidin-1-ylethoxy)benzyl]-1,3-benzothiazol-2-amine;
N-[3-(2-morpholin-4-ylethoxy)benzyl]-6-(7H-pyrrolo[2,3-d]pyrimidin-4-yl)-1,3-benzothiazol-2-amine;
6-(2-fluoropyridin-4-yl)-N-[3-(2-morpholin-4-ylethoxy)benzyl]-1,3-benzothiazol-2-amine;
6-(2-fluoropyridin-4-yl)-N-(2-piperidin-1-ylethyl)-1,3-benzothiazol-2-amine;
6-(2-fluoropyridin-4-yl)-N-(3-methoxybenzyl)-N-(2-piperidin-1-ylethyl)-1,3-benzothiazol-2-amine;
methyl 3-{[(6-pyridin-4-yl-1,3-benzothiazol-2-yl)amino]methyl}benzoate;
N-(3-methoxybenzyl)-N′,N′-dimethyl-N-(6-pyridin-4-yl-1,3-benzothiazol-2-yl)ethane-1,2-diamine;
N-[2-(dimethylamino)ethyl]-N-methyl-3-{[(6-pyridin-4-yl-1,3-benzothiazol-2-yl)amino]methyl}benzamide;
N-[2-(dimethylamino)ethyl]-3-{[(6-pyridin-4-yl-1,3-benzothiazol-2-yl)amino]methyl}benzamide;
N-(2-hydroxyethyl)-3-{[(6-pyridin-4-yl-1,3-benzothiazol-2-yl)amino]methyl}benzamide;
N-(2-morpholin-4-ylethyl)-3-{[(6-pyridin-4-yl-1,3-benzothiazol-2-yl)amino]methyl}benzamide;
N-(2-hydroxyethyl)-N-methyl-3-{[(6-pyridin-4-yl-1,3-benzothiazol-2-yl)amino]methyl}benzamide;
N-[(1R)-1-(3-propoxyphenyl)ethyl]-6-pyridin-4-yl-1,3-benzothiazol-2-amine;
2-(2-phenylpyrrolidin-1-yl)-6-pyridin-4-yl-1,3-benzothiazole;
6-pyridin-4-yl-2-(2-thien-2-ylpyrrolidin-1-yl)-1,3-benzothiazole;
2-[2-(4-fluorophenyl)pyrrolidin-1-yl]-6-pyridin-4-yl-1,3-benzothiazole;
2-(3-phenylpyrrolidin-1-yl)-6-pyridin-4-yl-1,3-benzothiazole;
2-[2-(5-chlorothien-2-yl)pyrrolidin-1-yl]-6-pyridin-4-yl-1,3-benzothiazole;
2-[2-(3-methoxyphenyl)pyrrolidin-1-yl]-6-pyridin-4-yl-1,3-benzothiazole;
6-pyridin-4-yl-2-[2-(1,3-thiazol-4-yl)pyrrolidin-1-yl]-1,3-benzothiazole;
2-(2-benzylpyrrolidin-1-yl)-6-pyridin-4-yl-1,3-benzothiazole;
2-[2-(5-methylthien-2-yl)pyrrolidin-1-yl]-6-pyridin-4-yl-1,3-benzothiazole;
2-[2-(3-fluorophenyl)pyrrolidin-1-yl]-6-pyridin-4-yl-1,3-benzothiazole;
2-[2-(3-chlorophenyl)pyrrolidin-1-yl]-6-pyridin-4-yl-1,3-benzothiazole;
6-pyridin-4-yl-2-[(2S)-2-thien-2-ylpyrrolidin-1-yl]-1,3-benzothiazole;
6-pyridin-4-yl-2-[(2R)-2-thien-2-ylpyrrolidin-1-yl]-1,3-benzothiazole;
3-[1-(6-pyridin-4-yl-1,3-benzothiazol-2-yl)pyrrolidin-2-yl]phenol;
3-{1-[6-(1H-pyrrolo[2,3-b]pyridin-4-yl)-1,3-benzothiazol-2-yl]pyrrolidin-2-yl}phenol;
2-{2-[3-(2-morpholin-4-ylethoxy)phenyl]pyrrolidin-1-yl}-6-pyridin-4-yl-1,3-benzothiazole;
2-{2-[3-(2-piperidin-1-ylethoxy)phenyl]pyrrolidin-1-yl}-6-pyridin-4-yl-1,3-benzothiazole;
3-[1-(6-pyrimidin-4-yl-1,3-benzothiazol-2-yl)pyrrolidin-2-yl]phenol;
3-{1-[6-(2-fluoropyridin-4-yl)-1,3-benzothiazol-2-yl]pyrrolidin-2-yl}phenol;
N-(3-fluorobenzyl)-5-pyridin-4-yl-1,3-benzothiazol-2-amine; and
N-benzyl-6-pyridin-4-yl-1,3-benzoxazol-2-amine.

17. A pharmaceutical composition comprising a therapeutically effective amount of a compound of claim (I), in combination with a pharmaceutically acceptable carrier.

18. A method for treating a disorder susceptible to treatment with ROCK modulators, said method comprising administering therapeutically effective amount of at least one compound of claim 1 or pharmaceutically acceptable salt thereof, to a subject in need thereof.

19. A method for treating a disease or a disorder in a mammal in need thereof comprising administering to the mammal therapeutically effective amount of at least one compound of claim 1 or a pharmaceutically acceptable salt thereof, wherein said disease or disorder is selected from the group consisting of hypertension, chronic and congestive heart failure, cardiac hypertrophy, chronic renal failure, cerebral vasospasm, pulmonary hypertension, ocular hypertension, cancer, tumor metastasis, asthma, male erectile dysfunctions, female sexual dysfunctions, over-active bladder syndrome, preterm labor, restenosis, atherosclerosis, neuronal injury, spinal cord injuries, traumatic brain injury and stroke, Parkinson's disease, Alzheimer disease, Huntington's disease, spinal muscular atrophy, amyotrophic lateral sclerosis, multiple sclerosis, encephalomyelitis, pain, rheumatoid arthritis, osteoarthritis, osteoporosis, irritable bowel syndrome, inflammatory bowel disease, HIV-1 encephalitis, diabetes, insulin resistance, ischemic CNS disorders, vascular or AD type dementia, glaucoma, psoriasis, retinopathy, benign prostatic hypertrophy, psychiatric disorders, depression, schizophrenia, obsessive compulsive disorder, bipolar disorder, epilepsy and seizure disorders, ischemia-reperfusion injury, myocardial infarct size and myocardial fibrosis, and diseases caused by viral and bacterial infections.

20. The method of claim 19 wherein the disease or disorder is selected from the group consisting of pain, asthma, cognitive dysfunctions, multiple sclerosis, cancer, rheumatoid arthritis, and spinal cord injuries.
